(19) United States
(12) Patent Application Publication
(10) Pub. No.: US 2003/0013699 A1
(43) Pub. Date: Jan. 16, 2003

(54) Methods for treating alzheimer's disease and/or regulating levels of amyloid beta peptides in a subject

(76) Inventors: Harry R. Davis, Bekeley Heights, NJ (US); Eric McFee Parker, Scotch Plains, NJ (US); Margaret van Heek, Scotch Plains, NJ (US); Gwendolyn T. Wong, Westfield, NJ (US); Laura B. Merkel, Princeton, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 10/154,106

(22) Filed: May 22, 2002

Related U.S. Application Data
(60) Provisional application No. 60/323,911, filed on Sep. 21, 2001.
(60) Provisional application No. 60/293,651, filed on May 25, 2001.

(51) Int. Cl. A61K 31/397

(52) U.S. Cl. 514/210.02

(57) ABSTRACT

Methods of preventing, treating, or ameliorating symptoms of Alzheimer's Disease, regulating production or levels of amyloid β (Aβ) peptides and/or regulating the amount of ApoE isoform 4 in the bloodstream and/or brain are provided which include the step of administering to a subject in need of such treatment an effective amount of a composition including at least one compound represented by Formulae (I-X) disclosed herein.

### CROSS-REFERENCE TO RELATED APPLICATIONS

This application claims the benefit of priority from U.S. Provisional Patent Application Serial No. 60/293,651, filed May 25, 2001 and U.S. Provisional Patent Application Serial No. 60/323,911, filed Sep. 21, 2001, each incorporated herein by reference.

### FIELD OF THE INVENTION

The present invention relates to methods for treating Alzheimer's Disease, regulating levels of amyloid β (Aβ) peptides and/or regulating the amount of ApoE isoform 4 in the bloodstream and/or brain of a subject by administering a composition comprising an effective amount of at least one of the compounds of Formulae I-X below.

### BACKGROUND OF THE INVENTION

Alzheimer's Disease (“AD”) is a neurodegenerative brain disease that is a major cause of dementia among the elderly. Symptoms of AD can include progressive loss of learning and memory functions, personality changes, neuromuscular changes, seizures and occasionally psychotic behavior. AD is characterized pathologically by the extracellular accumulation of senile plaques in various brain regions and vascular walls, as well as by the intraneuronal accumulation of neurofibrillary tangles in various brain regions. The main constituents of senile plaques are amyloid β (Aβ) peptides, such as for example 40-42 amino acid Aβ peptides, which are formed by proteolytic processing of the β-amyloid precursor protein (β-APP) by two enzymes known as β-secretase and γ-secretase.

The apolipoprotein E isoform 4 (ApoE isoform 4) is a major genetic risk factor for AD. PCT Patent Application No. WO 95/06470 discloses administration of an HMG-CoA reductase inhibitor (statin) to regulate levels of (ApoE isoform 4) in humans to prevent and treat Alzheimer's Disease.

A normal cellular function of ApoE is uptake and delivery of lipids. The ApoE isoform correlates with an increased risk for atherosclerosis, increased amyloid plaque deposition and increased risk of AD. K. Fassbender et al., “Simvastatin Strongly Reduces Levels of Alzheimer's Disease β-amyloid peptides Aβ42 and Aβ40 in vitro and in vivo”, PNAS 98: 5856-5861 (2001).

PCT Patent Application WO 00/28981 discloses at page 3 that patients possessing the ApoE isoform 4 have an increased risk for AD, as well as elevated levels of cholesterol and increased risk for heart disease.

PCT WO 00/28981 also discloses methods for treating AD using HMG-CoA reductase inhibitors, but notes at page 6 that “it was unexpectedly discovered that while some HGM (sic) CoA reductase inhibitors exhibit a dramatic reduction in degree and prevalence of AD, patients taking other types of drugs used to treat cardiovascular disorders, such as beta blockers, furosemide, and captopril, did not show any significant reduction in the prevalence or degree of AD.”

U.S. Pat. No. 6,080,778 (col. 1, lines 39-41) points out that known genetic causes of AD can account for only a small proportion of the total number of cases and that most cases of AD are sporadic (i.e., without a known genetic cause) and occur in the elderly. This patent discloses methods for decreasing the production of Aβ peptides by administering a composition which decreases blood cholesterol levels to a person with elevated cholesterol levels who is at risk of, or exhibits symptoms of, Alzheimer's Disease. The disclosed methods include administration of compounds which increase uptake of cholesterol by the liver (e.g., HMG CoA reductase inhibitors), compounds which block endogenous cholesterol production, compounds which prevent uptake of dietary cholesterol (e.g., bile acid binding resins and fibrates), and combinations of any of these which are effective in lowering blood cholesterol levels (see col. 1, line 58—col. 2, line 5). Fassbender et al. disclose that use of simvastatin and lovastatin, alone or in combination with methyl-β-cyclodextrin, can reduce intracellular and secreted Aβ levels in vitro and that treatment of animals with simvastatin reduces brain and cerebrospinal fluid levels of Aβ in vivo.

U.S. Pat. No. 6,071,899 discloses compounds of Formula (I):

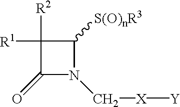

having substituents as defined therein, which may have a general application in any disorder that involves endothelial dysfunction, such as atherosclerosis, or may have a general application in any disorder that involves lipid peroxidation in conjunction with enzyme activity, including inflammatory conditions of the brain such as Alzheimer's Disease (see col. 5, lines 16-29).

PCT Patent Application WO 99/38498 discloses methods for preventing or treating AD by administering a plasma-triglyceride level-lowering agent (e.g., fibrates), optionally in combination with a cholesterol level-lowering agent such as statins, bile acid sequestrants or agents that block intestinal cholesterol absorption (e.g., β-sitosterol, SCH 48461 ((3R,4S)-1,4-bis-(4-methoxyphenyl)-3-(3-phenylpropyl)-2-azetidinone), CP-148,623, saponins, neomycin and ACAT inhibitors).

U.S. Pat. Nos. 5,767,115, 5,624,920, 5,688,990, 5,656,624 and 5,688,787, respectively, disclose hydroxy-substituted azetidinone compounds and substituted β-lactam compounds useful for lowering cholesterol and/or in inhibiting the formation of cholesterol-containing lesions in mammalian arterial walls, but does not disclose treatment of Alzheimer's Disease.

Despite recent improvements in the treatment of Alzheimer's disease, there remains a need in the art for improved methods of treatment which are effective and avoid undesirable side effects.

### SUMMARY OF THE INVENTION

In one embodiment, the present invention provides a method of preventing, treating, or ameliorating symptoms of Alzheimer's Disease comprising the step of administering to a subject in need of such treatment an effective amount of a composition comprising at least one compound represented by Formula (I):

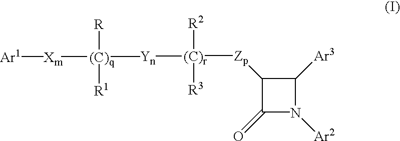

or a pharmaceutically acceptable salt thereof or solvate thereof, wherein:

Ar¹ and Ar² are independently selected from the group consisting of aryl and R⁴-substituted aryl;

Ar³ is aryl or R⁵-substituted aryl;

X, Y and Z are independently selected from the group consisting of —CH₂—, —CH(lower alkyl)- and —C(dilower alkyl)-;

R and R² are independently selected from the group consisting of —OR⁶, —O(CO)R⁶, —O(CO)OR⁹ and —O(CO)NR⁶R⁷;

R¹ and R³ are independently selected from the group consisting of hydrogen, lower alkyl and aryl;

q is 0 or 1;

r is 0 or 1;

m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;

R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl to prevent, treat, or ameliorate symptoms of Alzheimer's Disease in the subject.

In another embodiment, a method of preventing, treating, or ameliorating symptoms of Alzheimer's Disease is provided which comprises the step of administering to a subject in need of such treatment an effective amount of a composition comprising a compound represented by Formula (II) below:

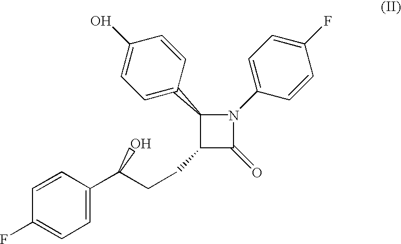

or a pharmaceutically acceptable salt or solvate of the compound of Formula (II) to prevent, treat, or ameliorate symptoms of Alzheimer's Disease in the subject.

In yet another embodiment, the present invention provides a method of preventing, treating, or ameliorating symptoms of Alzheimer's Disease comprising the step of administering to a subject in need of such treatment an effective amount of a composition comprising at least one compound represented by Formula (III):

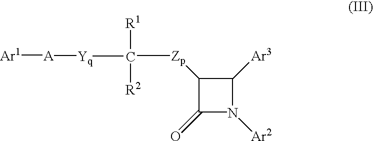

or a pharmaceutically acceptable salt thereof or solvate thereof, wherein, in Formula (III) above:

Ar¹ is R³-substituted aryl;

Ar² is R⁴-substituted aryl;

Ar³ is R⁵-substituted aryl;

Y and Z are independently selected from the group consisting of —CH₂—, —CH(lower alkyl)- and —C(dilower alkyl)-;

A is selected from —O—, —S—, —S(O)— or —S(O)₂—;

R¹ is selected from the group consisting of —OR⁶, —O(CO)R⁶, —O(CO)OR⁹ and —O(CO)NR⁶R⁷; R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;

R³ and R⁴ are independently 1-3 substituents independently selected from the group consisting of R⁵, hydrogen, p-lower alkyl, aryl, —NO₂, —CF₃ and p-halogeno;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl to prevent, treat, or ameliorate symptoms of Alzheimer's Disease in the subject.

In yet another embodiment, the present invention provides a method of preventing, treating, or ameliorating symptoms of Alzheimer's Disease comprising the step of administering to a subject in need of such treatment an effective amount of a composition comprising at least one compound represented by Formula (IV):

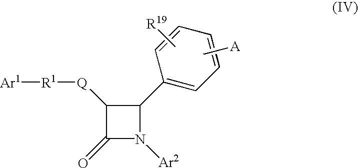

or a pharmaceutically acceptable salt thereof or solvate thereof, wherein, in Formula (IV) above:

A is selected from the group consisting of R²-substituted heterocycloalkyl, R²-substituted heteroaryl, R²-substituted benzofused heterocycloalkyl, and R²-substituted benzofused heteroaryl;

Ar¹ is aryl or R³-substituted aryl;

Ar² is aryl or R⁴-substituted aryl;

Q is a bond or, with the 3-position ring carbon of the azetidinone, forms the spiro group

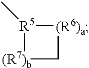

and

R¹ is selected from the group consisting of:

—(CH₂)_{q}—, wherein q is 2-6, provided that when Q forms a spiro ring, q can also be zero or 1;

—(CH₂)ₑ-G-(CH₂)ᵣ—, wherein G is —O—, —C(O)—, phenylene, —NR⁸— or —S(O)₀₋₂, e is 0-5 and r is 0-5, provided that the sum of e and r is 1-6;

—(C₂-C₆ alkenylene)-; and

—(CH₂)_{f}—V—(CH₂)_{g}—, wherein V is C₃-C₆ cycloalkylene, f is 1-5 and g is 0-5, provided that the sum of f and g is 1-6;

R⁵ is selected from:

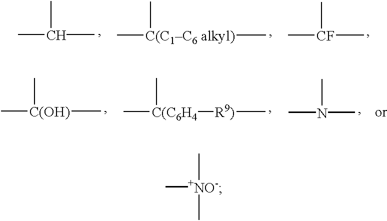

R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)- group;

a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷'s can be the same or different;

and when Q is a bond, R¹ also can be selected from:

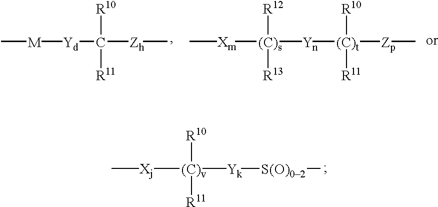

where M is —O—, —S—, —S(O)— or —S(O)₂—;

X, Y and Z are independently selected from the group consisting of —CH₂—, —CH(C₁-C₆ alkyl)- and —C(di-(C₁-C₆) alkyl);

R¹⁰ and R¹² are independently selected from the group consisting of —OR¹⁴, —O(CO)R¹⁴, —O(CO)OR¹⁶ and —O(CO)NR¹⁴R¹⁵;

R¹¹ and R¹³ are independently selected from the group consisting of hydrogen, (C₁-C₆)alkyl and aryl; or R¹⁰ and R¹¹ together are ═O, or R¹² and R¹³ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R¹⁶, —SO₂NR¹⁴R¹⁵ and —(C₁-C₆ alkylene)COOR¹⁴; when R² is a substituent on a heterocycloalkyl ring, R² is as defined, or is ═O or

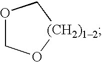

and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸,

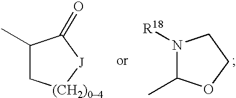

wherein J is —O—, —NH—, —NR¹⁸— or —CH₂—;

R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR¹⁶, —NR¹⁴(CO)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;

R⁸ is hydrogen, (C₁-C₆)alkyl, aryl (C₁-C₆)alkyl, —C(O)R¹⁴ or —COOR¹⁴;

R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;

R¹⁴ and R¹⁵ are independently selected from the group consisting of hydrogen, (C₁-C₆)alkyl, aryl and aryl-substituted (C₁-C₆)alkyl;

R¹⁶ is (C₁-C₆)alkyl, aryl or R¹⁷-substituted aryl;

R¹⁸ is hydrogen or (C₁-C₆)alkyl; and

R¹⁹ is hydrogen, hydroxy or (C₁-C₆)alkoxy, to prevent, treat, or ameliorate symptoms of Alzheimer's Disease in the subject.

In another embodiment, the present invention provides a method of preventing, treating, or ameliorating symptoms of Alzheimer's Disease comprising the step of administering to a subject in need of such treatment an effective amount of a composition comprising at least one compound represented by Formula (V):

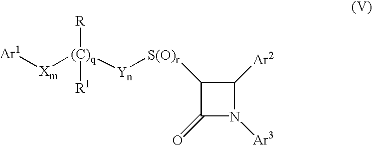

or a pharmaceutically acceptable salt thereof or solvate thereof, wherein, in Formula (V) above:

Ar¹ is aryl, R¹⁰-substituted aryl or heteroaryl;

Ar² is aryl or R⁴-substituted aryl;

Ar³ is aryl or R⁵-substituted aryl;

X and Y are independently selected from the group consisting of —CH₂—, —CH(lower alkyl)- and —C(dilower alkyl)-;

R is —OR⁶, —O(CO)R⁶, —O(CO)OR⁹ or —O(CO)NR⁶R⁷; R¹ is hydrogen, lower alkyl or aryl; or R and R¹ together are ═O;

q is 0 or 1;

r is 0, 1 or 2;

m and n are independently 0, 1, 2, 3, 4 or 5; provided that the sum of m, n and q is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl;

R⁹ is lower alkyl, aryl or aryl-substituted lower alkyl; and

R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, —CF₃, —CN, —NO₂ and halogen to prevent, treat, or ameliorate symptoms of Alzheimer's Disease in the subject.

In yet another embodiment, the present invention provides a method of preventing, treating, or ameliorating symptoms of Alzheimer's Disease comprising the step of administering to a subject in need of such treatment an effective amount of a composition comprising at least one compound represented by Formula (VI):

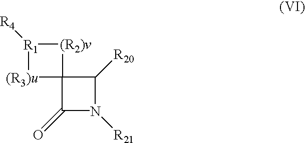

or a pharmaceutically acceptable salt thereof or solvate thereof, wherein:

R₁ is

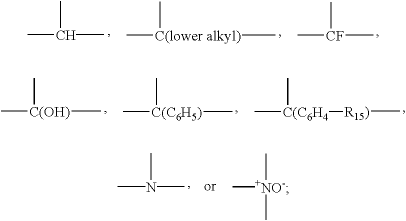

R₂ and R₃ are independently selected from the group consisting of: —CH₂—, —CH(lower alkyl)-, —C(di-lower alkyl)-, —CH═CH— and —C(lower alkyl)=CH—; or R₁ together with an adjacent R₂, or R₁ together with an adjacent R₃, form a —CH═CH— or a —CH═C(lower alkyl)- group;

u and v are independently 0, 1, 2 or 3, provided both are not zero; provided that when R₂ is —CH═CH— or —C(lower alkyl)=CH—, v is 1; provided that when R₃ is —CH═CH— or —C(lower alkyl)=CH—, u is 1; provided that when v is 2 or 3, the R₂'s can be the same or different; and provided that when u is 2 or 3, the R₃'s can be the same or different;

R₄ is selected from B—(CH₂)ₘC(O)—, wherein m is 0, 1, 2, 3, 4 or 5;

B—(CH₂)_{q}—, wherein q is 0, 1, 2, 3, 4, 5 or 6;

B—(CH₂)ₑ-Z-(CH₂)ᵣ—, wherein Z is —O—, —C(O)—, phenylene, —N(R₈)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6;

B—(C₂-C₆ alkenylene)-;

B—(C₄-C₆ alkadienylene)-;

B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6;

B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6;

B—(CH₂)ₜ—V—(C₂-C₆ alkenylene)- or

B—(C₂-C₆ alkenylene)—V—(CH₂)ₜ—, wherein V and t are as defined above, provided that the sum of t and the number of carbon atoms in the alkenylene chain is 2, 3, 4, 5 or 6;

B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or

T-(CH₂)ₛ—, wherein T is cycloalkyl of 3-6 carbon atoms and s is 0, 1, 2, 3, 4, 5 or 6; or

R₁ and R₄ together form the group

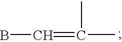

B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or

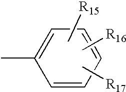

W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R₇-benzyl, benzyloxy, R₇-benzyloxy, phenoxy, R₇-phenoxy, dioxolanyl, NO₂, —N(R₈)(R₉), N(R₈)(R₉)-lower alkylene-, N(R₈)(R₉)-lower alkylenyloxy-, OH, halogeno, —CN, —N₃, —NHC(O)OR₁₀, —NHC(O)R₁₀, R₁₁O₂SNH—, (R₁₁O₂S)₂N—, —S(O)₂NH₂, —S(O)₀₋₂R₈, tert-butyldimethyl-silyloxymethyl, —C(O)R₁₂, —COOR₁₉, —CON(R₈)(R₉), —CH═CHC(O)R₁₂, -lower alkylene-C(O)R₁₂, R₁₀C(O)(lower alkylenyloxy)-, N(R₈)(R₉)C(O)(lower alkylenyloxy)- and

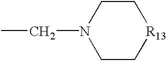

for substitution on ring carbon atoms, and the substituents on the substituted heteroaryl ring nitrogen atoms, when present, are selected from the group consisting of lower alkyl, lower alkoxy, —C(O)OR₁₀, —C(O)R₁₀, OH, N(R₈)(R₉)-lower alkylene-, N(R₈)(R₉)-lower alkylenyloxy-, —S(O)₂NH₂ and 2-(trimethylsilyl)-ethoxymethyl;

R₇ is 1-3 groups independently selected from the group consisting of lower alkyl, lower alkoxy, —COOH, NO₂, —N(R₈)(R₉), OH, and halogeno;

R₈ and R₉ are independently selected from H or lower alkyl;

R₁₀ is selected from lower alkyl, phenyl, R₇-phenyl, benzyl or R₇-benzyl;

R₁₁ is selected from OH, lower alkyl, phenyl, benzyl, R₇-phenyl or R₇-benzyl;

R₁₂ is selected from H, OH, alkoxy, phenoxy, benzyloxy,

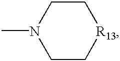

—N(R₈)(R₉), lower alkyl, phenyl or R₇-phenyl;

R₁₃ is selected from —O—, —CH₂—, —NH—, —N(lower alkyl)- or —NC(O)R₁₉;

R₁₅, R₁₆ and R₁₇ are independently selected from the group consisting of H and the groups defined for W; or R₁₅ is hydrogen and R₁₆ and R₁₇, together with adjacent carbon atoms to which they are attached, form a dioxolanyl ring;

R₁₉ is H, lower alkyl, phenyl or phenyl lower alkyl; and

R₂₀ and R₂₁ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above, to prevent, treat, or ameliorate symptoms of Alzheimer's Disease in the subject.

In yet another embodiment, the present invention provides a method of preventing, treating, or ameliorating symptoms of Alzheimer's Disease comprising the step of administering to a subject in need of such treatment an effective amount of a composition comprising at least one compound represented by Formula (VII):

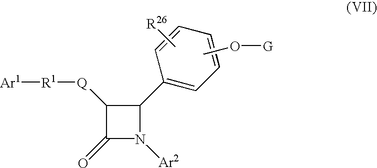

or a pharmaceutically acceptable salt thereof or solvate thereof, wherein, in Formula (VII) above,

R²⁶ is H or OG¹;

G and G¹ are independently selected from the group consisting of

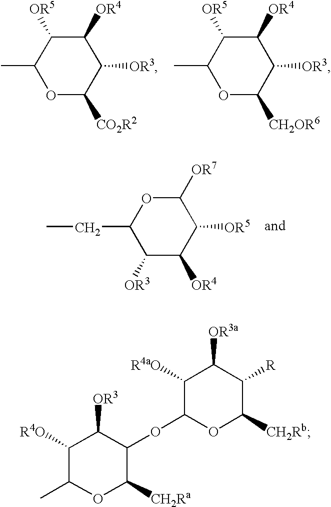

provided that when R²⁶ is H or OH, G is not H;

R, R^{a} and R^{b} are independently selected from the group consisting of H, —OH, halogeno, —NH₂, azido, (C₁-C₆)alkoxy(C₁-C₆)-alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is selected from the group consisting of H and (C₁-C₄)alkyl;

T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is a bond or, with the 3-position ring carbon of the azetidinone, forms the spiro group

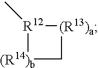

and

R¹ is selected from the group consisting of

—(CH₂)_{q}—, wherein q is 2-6, provided that when Q forms a spiro ring, q can also be zero or 1;

—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;

—(C₂-C₆)alkenylene-; and

—(CH₂)_{f}—V—(CH₂)_{g}—, wherein V is C₃-C₆ cycloalkylene, f is 1-5 and g is 0-5, provided that the sum of f and g is 1-6;

R¹² is

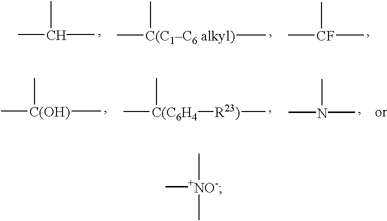

R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero;

provided that when R¹³ is —CH═CH— or —C(C₁-C₆ alkyl)=CH—, a is 1;

provided that when R¹⁴ is —CH═CH— or —C(C₁-C₆ alkyl)=CH—, b is 1;

provided that when a is 2 or 3, the R¹³'s can be the same or different; and

provided that when b is 2 or 3, the R¹⁴'s can be the same or different;

and when Q is a bond, R¹ also can be:

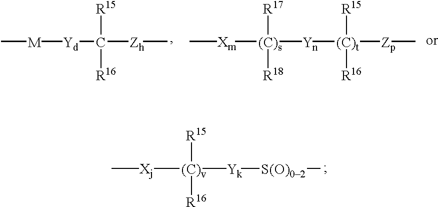

M is —O—, —S—, —S(O)— or —S(O)₂—;

X, Y and Z are independently selected from the group consisting of —CH₂—, —CH(C₁-C₆)alkyl- and —C(di-(C₁-C₆)alkyl);

R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;

R¹⁵ and R¹⁷ are independently selected from the group consisting of —OR¹⁹, —O(CO)R¹⁹, —O(CO)OR²¹ and —O(CO)NR¹⁹R²⁰;

R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;

d is 1, 2 or 3;

h is 0, 1, 2, 3 or 4;

s is 0 or 1; t is 0 or 1; m, n and p are independently 0-4;

provided that at least one of s and t is 1, and the sum of m, n, p, s and t is 1-6;

provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

and when Q is a bond and R¹ is

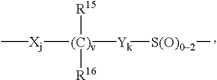

Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —COOR¹⁹;

R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and

R²⁵ is H, —OH or (C₁-C₆)alkoxy, to prevent, treat, or ameliorate symptoms of Alzheimer's Disease in the subject.

In yet another embodiment, the present invention provides a method of preventing, treating, or ameliorating symptoms of Alzheimer's Disease comprising the step of administering to a subject in need of such treatment an effective amount of a composition comprising at least one compound represented by Formula (VIII):

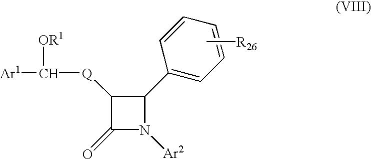

or a pharmaceutically acceptable salt or solvate thereof, wherein

R²⁶ is selected from the group consisting of:

a) OH;

b) OCH₃;

c) fluorine and

d) chlorine.

R¹ is selected from the group consisting of

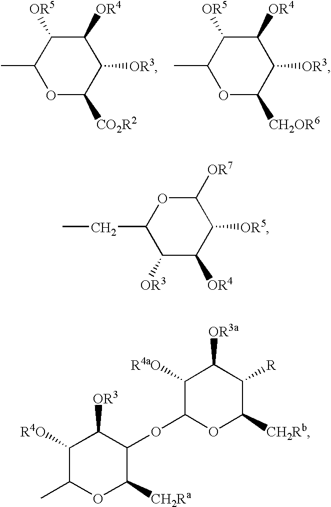

—SO₃H; natural and unnatural amino acids.

R, R^{a} and R^{b} are independently selected from the group consisting of H, —OH, halogeno, —NH₂, azido, (C₁-C₆)alkoxy(C₁-C₆)-alkoxy and —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is independently selected form the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is independently selected from the group consisting of H and (C₁-C₄)alkyl;

T is independently selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of H, halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is —(CH₂)_{q}—, wherein q is 2-6, or, with the 3-position ring carbon of the azetidinone, forms the spiro group

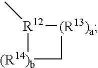

R¹² is

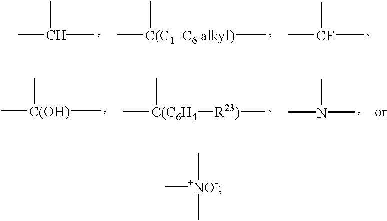

R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, the R¹³'s can be the same or different; and provided that when b is 2 or 3, the R¹⁴'s can be the same or different;

R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —COOR¹⁹;

R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and

R²⁵ is H, —OH or (C₁-C₆)alkoxy, to prevent, treat, or ameliorate symptoms of Alzheimer's Disease in the subject.

In yet another embodiment, the present invention provides a method of regulating the production of amyloid β peptide in a subject comprising the step of administering to a subject in need of such treatment an effective amount of a composition comprising at least one compound represented by Formulae (I-X), or a pharmaceutically acceptable salt or solvate thereof, to regulate the production of amyloid β peptide in the subject.

In yet another embodiment, the present invention provides a method of regulating a level of one or more amyloid β peptides in a subject comprising the step of administering to a subject in need of such treatment an effective amount of a composition comprising at least one compound represented by Formulae (I-X), or a pharmaceutically acceptable salt or solvate thereof, to regulate a level of one or more amyloid β peptides in the bloodstream and/or the brain of the subject.

In yet another embodiment, the present invention provides a method of regulating the amount of ApoE isoform 4 in the bloodstream and/or brain of a subject comprising the step of administering to a subject in need of such treatment an effective amount of a composition comprising at least one compound represented by Formulae (I-X), to regulate the amount of ApoE isoform 4 in the bloodstream and/or brain of the subject.

In yet another embodiment, the present invention provides a use of at least one compound represented by Formulae (I-X) for manufacture of a medicament for the treatment of Alzheimer's Disease, regulating production of or level of at least one amyloid β peptide and/or regulating the amount of ApoE isoform 4 in the bloodstream and/or brain of a subject.

Other than in the operating examples, or where otherwise indicated, all numbers expressing quantities of ingredients, reaction conditions, and so forth used in the specification and claims are to be understood as being modified in all instances by the term “about.”

### DETAILED DESCRIPTION

In one embodiment, the present invention provides methods of preventing Alzheimer's Disease (“AD”), treating AD, or lessening the number or severity of or ameliorating the symptoms of AD in a subject comprising the step of administering to a subject in need of such treatment an effective amount of a composition comprising at least one compound represented by Formulae (I-X) described below to prevent, treat, and/or ameliorate the symptoms of AD in the subject.

In another embodiment, the present invention provides methods of regulating production of or levels of one or more amyloid β peptides in a subject comprising the step of administering to a subject in need of such treatment an effective amount of a composition comprising at least one compound represented by Formulae (I-X) described below to regulate levels of one or more amyloid β peptides in the bloodstream and/or the brain of the subject.

In another embodiment, the present invention provides methods of regulating levels of ApoE isoform 4 in a subject comprising the step of administering to a subject in need of such treatment an effective amount of a composition comprising at least one compound represented by Formulae (I-X) described below to regulate levels of ApoE isoform 4 in the bloodstream and/or the brain of the subject.

As discussed above, AD is the most common cause of dementia in the elderly and can be pathologically characterized by the accumulation of senile plaques comprising amyloid β peptides in the extracellular space of various brain regions and in vascular walls. As used herein, “amyloid β peptide” means Aβ peptides which are derived from the larger β-amyloid precursor protein (βAPP) through the endopeptidase action of β and γ secretases. Non-limiting examples of such Aβ peptides include those that contain 40 or 42 amino acids.

In familial forms of AD, the pathological appearance of Aβ peptides in the brain is driven by the presence of mutations in the βAPP gene or in the genes coding for the proteins presenilin 1 and 2. The Apolipoprotein E type 4 allele (which encodes the protein ApoE isoform 4) is genetically associated with common late onset sporadic forms of AD.

The term “effective amount” means that amount or dosage of a compound or a combination of compounds represented by Formulae (I-X) described below, that will elicit a biological or medical response of a tissue, system or subject that is being sought by the administrator (such as a researcher or doctor) which includes amelioration or alleviation of one or more of the symptoms of the condition or disease (such as Alzheimer's Disease, regulating production of or regulating or reducing levels of one or more amyloid β peptides and/or regulating or reducing levels of ApoE isoform 4 in the bloodstream and/or the brain) being treated and/or the prevention, slowing or halting of progression of the condition. As used herein, the phrase “ameliorating the symptoms of AD” means alleviating, reducing or eliminating one or more of the symptoms experienced by a subject afflicted with AD, including one or more of the following symptoms: progressive loss of learning and memory functions, personality changes, neuromuscular changes, seizures and psychotic behavior.

Examples of suitable dosages are discussed in detail below.

Examples of suitable subjects that can be treated according to the methods of the present invention include mammals, such as humans or dogs, and other animals.

As used herein, “combination therapy” or “therapeutic combination” means the administration of two or more compounds represented by Formulae (I-X) or administration of one or more compounds represented by Formulae (I-X) with cholesterol biosynthesis inhibitors and/or lipid-lowering agents and/or other Alzheimer's disease treatments different from compounds represented by Formulae (I-X) discussed below, to prevent or treat Alzheimer's Disease, reduce levels of one or more amyloid β peptides, regulate production of amyloid β peptides and/or regulate levels of ApoE isoform 4 in the bloodstream and/or the brain. Such administration includes coadministration of these therapeutic agents in a substantially simultaneous manner, such as in a single tablet or capsule having a fixed ratio of active ingredients or in multiple, separate capsules for each therapeutic agent. Also, such administration includes use of each type of therapeutic agent in a sequential manner. In either case, the treatment using the combination therapy will provide beneficial effects in treating the condition.

A potential advantage of the combination therapy disclosed herein may be a reduction in the required amount of an individual therapeutic compound or the overall total amount of therapeutic compounds that are effective in treating the condition. By using a combination of therapeutic agents, the side effects of the individual compounds can be reduced as compared to a monotherapy, which can improve subject compliance. Also, therapeutic agents can be selected to provide a broader range of complimentary effects or complimentary modes of action.

In one embodiment, the methods of the present invention comprise the step of administering to a subject in need of such treatment an effective amount of a composition comprising one or more compounds represented by Formula (I) below:

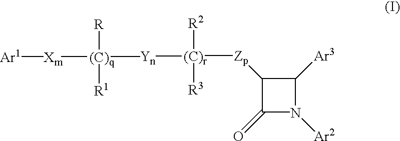

or a pharmaceutically acceptable salt thereof or solvate thereof. In Formula (I) above:

Ar¹ and Ar² are independently selected from the group consisting of aryl and R⁴-substituted aryl;

Ar³ is aryl or R⁵-substituted aryl;

X, Y and Z are independently selected from the group consisting of —CH₂—, —CH(lower alkyl)- and —C(dilower alkyl)-;

R and R² are independently selected from the group consisting of —OR⁶, —O(CO)R⁶, —O(CO)OR⁹ and —O(CO)NR⁶R⁷;

R¹ and R³ are independently selected from the group consisting of hydrogen, lower alkyl and aryl;

q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;

R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl.

Preferably, R⁴ is 1-3 independently selected substituents, and R⁵ is preferably 1-3 independently selected substituents.

As used herein, the term “alkyl” or “lower alkyl” means straight or branched alkyl chains having from 1 to 6 carbon atoms and “alkoxy” means alkoxy groups having 1 to 6 carbon atoms. Non-limiting examples of lower alkyl groups include, for example methyl, ethyl, propyl, and butyl groups.

“Alkenyl” means straight or branched carbon chains having one or more double bonds in the chain, conjugated or unconjugated. Similarly, “alkynyl” means straight or branched carbon chains having one or more triple bonds in the chain. Where an alkyl, alkenyl or alkynyl chain joins two other variables and is therefore bivalent, the terms alkylene, alkenylene and alkynylene are used.

“Cycloalkyl” means a saturated carbon ring of 3 to 6 carbon atoms, while “cycloalkylene” refers to a corresponding bivalent ring, wherein the points of attachment to other groups include all positional isomers.

“Halogeno” refers to fluorine, chlorine, bromine or iodine radicals.

“Aryl” means phenyl, naphthyl, indenyl, tetrahydronaphthyl or indanyl.

“Phenylene” means a bivalent phenyl group, including ortho, meta and para-substitution.

The statements wherein, for example, R, R¹, R² and R³ are said to be independently selected from a group of substituents, mean that R, R¹, R² and R³ are independently selected, but also that where an R, R¹, R² and R³ variable occurs more than once in a molecule, each occurrence is independently selected (e.g., if R is —OR⁶, wherein R⁶ is hydrogen, R² can be —OR⁶ wherein R⁶ is lower alkyl). Those skilled in the art will recognize that the size and nature of the substituent(s) will affect the number of substituents that can be present.

Compounds of the invention have at least one asymmetrical carbon atom and therefore all isomers, including enantiomers, stereoisomers, rotamers, tautomers and racemates of the compounds of Formula (I-X) (where they exist) are contemplated as being part of this invention. The invention includes d and I isomers in both pure form and in admixture, including racemic mixtures. Isomers can be prepared using conventional techniques, either by reacting optically pure or optically enriched starting materials or by separating isomers of a compound of the Formulae I-X. Isomers may also include geometric isomers, e.g., when a double bond is present.

Those skilled in the art will appreciate that for some of the compounds of the Formulas I-X, one isomer will show greater pharmacological activity than other isomers.

Compounds of the invention with an amino group can form pharmaceutically acceptable salts with organic and inorganic acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those in the art. The salt is prepared by contacting the free base form with a sufficient amount of the desired acid to produce a salt. The free base form may be regenerated by treating the salt with a suitable dilute aqueous base solution such as dilute aqueous sodium bicarbonate. The free base form differs from its respective salt form somewhat in certain physical properties, such as solubility in polar solvents, but the salt is otherwise equivalent to its respective free base forms for purposes of the invention.

Certain compounds of the invention are acidic (e.g., those compounds which possess a carboxyl group). These compounds form pharmaceutically acceptable salts with inorganic and organic bases. Examples of such salts are the sodium, potassium, calcium, aluminum, gold and silver salts. Also included are salts formed with pharmaceutically acceptable amines such as ammonia, alkyl amines, hydroxyalkylamines, N-methylglucamine and the like.

As used herein, “solvate” means a molecular or ionic complex of molecules or ions of solvent with those of solute (for example, one or more compounds of Formulae I-X, isomers of the compounds of Formulae I-X, or prodrugs of the compounds of Formulae I-X). Non-limiting examples of useful solvents include polar, protic solvents such as water and/or alcohols (for example methanol).

Prodrugs of the compounds of Formulae I-X are contemplated as being part of this invention. As used herein, “prodrug” means compounds that are drug precursors which, following administration to a subject, release the drug in vivo via some chemical or physiological process (e.g., a prodrug on being brought to the physiological pH or through enzyme action is converted to the desired drug form).

Preferred compounds of Formula (I) are those in which Ar¹ is phenyl or R⁴-substituted phenyl, more preferably (4-R⁴)-substituted phenyl. Ar² preferably phenyl or R⁴-substituted phenyl, more preferably (4-R⁴)-substituted phenyl. Ar³ is preferably R⁵-substituted phenyl, more preferably (4-R⁵)-substituted phenyl. When Ar¹ is (4-R⁴)-substituted phenyl, R⁴ is preferably a halogen. When Ar² and Ar³ are R⁴- and R⁵-substituted phenyl, respectively, R⁴ is preferably halogen or —OR⁶ and R⁵ is preferably —OR⁶, wherein R⁶ is lower alkyl or hydrogen. Especially preferred are compounds wherein each of Ar¹ and Ar² is 4-fluorophenyl and Ar³ is 4-hydroxyphenyl or 4-methoxyphenyl.

X, Y and Z are each preferably —CH₂—. R¹ and R³ are each preferably hydrogen. R and R² are preferably —OR⁶ wherein R⁶ is hydrogen, or a group readily metabolizable to a hydroxyl (such as —O(CO)R⁶, —O(CO)OR⁹ and —O(CO)NR⁶R⁷, defined above).

The sum of m, n, p, q and r is preferably 2, 3 or 4, more preferably 3. Preferred are compounds wherein m, n and r are each zero, q is 1 and p is 2.

Also preferred are compounds of Formula (I) in which p, q and n are each zero, r is 1 and m is 2 or 3. More preferred are compounds wherein m, n and r are each zero, q is 1, p is 2, Z is —CH₂— and R is —OR⁶, especially when R⁶ is hydrogen.

Also more preferred are compounds of Formula (I) wherein p, q and n are each zero, r is 1, m is 2, X is —CH₂— and R² is —OR⁶, especially when R⁶ is hydrogen.

Another group of preferred compounds of Formula (I) is that in which Ar¹ is phenyl or R⁴-substituted phenyl, Ar² is phenyl or R⁴-substituted phenyl and Ar³ is R⁵-substituted phenyl. Also preferred are compounds in which Ar¹ is phenyl or R⁴-substituted phenyl, Ar² is phenyl or R⁴-substituted phenyl, Ar³ is R⁵-substituted phenyl, and the sum of m, n, p, q and r is 2, 3 or 4, more preferably 3. More preferred are compounds wherein Ar¹ is phenyl or R⁴-substituted phenyl, Ar² is phenyl or R⁴-substituted phenyl, Ar³ is R⁵-substituted phenyl, and wherein m, n and r are each zero, q is 1 and p is 2, or wherein p, q and n are each zero, r is 1 and m is 2 or 3.

In a preferred embodiment, a compound of Formula (I) useful in the methods of the present invention is represented by Formula (II) (ezetimibe) below:

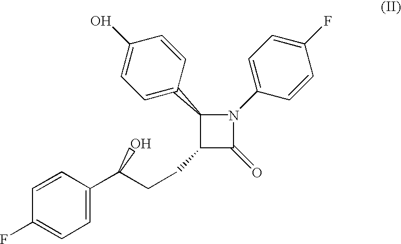

or a pharmaceutically acceptable salt or solvate thereof. The compound of Formula (II) can be in anhydrous or hydrated form.

Compounds of Formula I can be prepared by a variety of methods well known to those skilled in the art, for example such as are disclosed in U.S. Pat. Nos. 5,631,365, 5,767,115, 5,846,966, 6,207,822, U.S. patent application Ser. No. 10/105,710 filed Mar. 25, 2002 and PCT Patent Application WO 93/02048, each of which is incorporated herein by reference, and in the Example below. For example, suitable compounds of Formula I can be prepared by a method comprising the steps of:

(a) treating with a strong base a lactone of the Formula A or B:

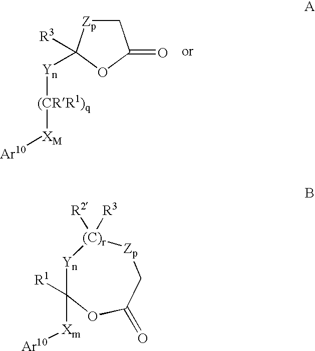

wherein R′ and R^{2′} are R and R², respectively, or are suitably protected hydroxy groups; Ar¹⁰ is Ar¹, a suitably protected hydroxy-substituted aryl or a suitably protected amino-substituted aryl; and the remaining variables are as defined above for Formula I, provided that in lactone of formula B, when n and r are each zero, p is 1-4;

(b) reacting the product of step (a) with an imine of the formula

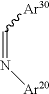

wherein Ar²⁰ is Ar², a suitably protected hydroxy-substituted aryl or a suitably protected amino-substituted aryl; and Ar³⁰ is Ar³, a suitably protected hydroxy-substituted aryl or a suitably protected amino-substituted aryl;

c) quenching the reaction with an acid;

d) optionally removing the protecting groups from R′, R^{2′}, Ar¹⁰, Ar²⁰ and Ar³⁰, when present; and

e) optionally functionalizing hydroxy or amino substituents at R, R², Ar¹, Ar² and Ar³.

Using the lactones shown above, compounds of Formula IA and IB are obtained as follows:

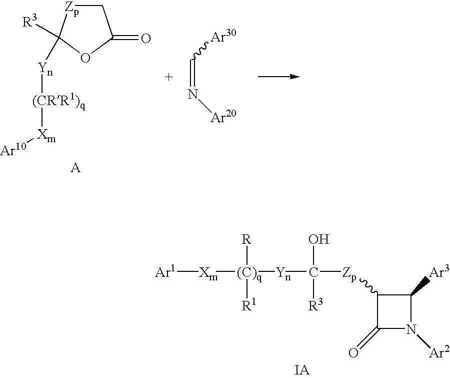

wherein the variables are as defined above; and

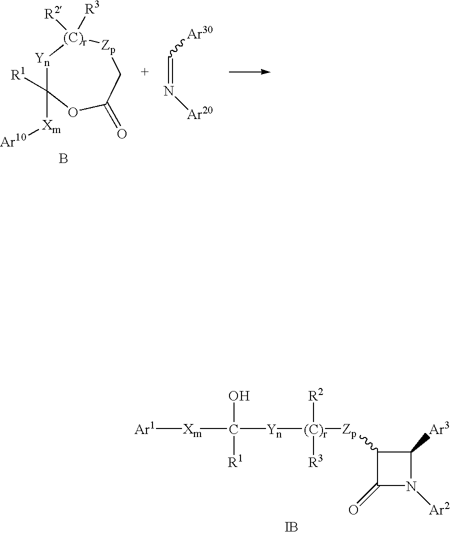

wherein the variables are as defined above.

Alternative compounds useful in the methods of the present invention are represented by Formula (III) below:

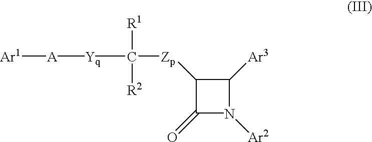

or a pharmaceutically acceptable salt thereof or solvate thereof, wherein, in Formula (III) above:

Ar¹ is R³-substituted aryl;

Ar² is R⁴-substituted aryl;

Ar³ is R⁵-substituted aryl;

Y and Z are independently selected from the group consisting of —CH₂—, —CH(lower alkyl)- and —C(dilower alkyl)-;

A is selected from —O—, —S—, —S(O)— or —S(O)₂—;

R¹ is selected from the group consisting of —OR⁶, —O(CO)R⁶, —O(CO)OR⁹ and —O(CO)NR⁶R⁷; R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;

R³ and R⁴ are independently 1-3 substituents independently selected from the group consisting of R⁵, hydrogen, p-lower alkyl aryl, —NO₂, —CF₃ and p-halogeno;

R⁶, R⁷ and R⁸ are independently selected from the group consisting of hydrogen, lower alkyl, aryl and aryl-substituted lower alkyl; and R⁹ is lower alkyl, aryl or aryl-substituted lower alkyl.

Preferred compounds of Formula III include those in which Ar¹ is R³-substituted phenyl, especially (4-R³)-substituted phenyl. Ar² is preferably R⁴-substituted phenyl, especially (4-R⁴)-substituted phenyl. Ar³ is preferably R⁵-substituted phenyl, especially (4-R⁵)-substituted phenyl. Mono-substitution of each of Ar¹, Ar² and Ar³ is preferred.

Y and Z are each preferably —CH₂—. R² is preferably hydrogen. R¹ is preferably —OR⁶ wherein R⁶ is hydrogen, or a group readily metabolizable to a hydroxyl (such as —O(CO)R⁶, —O(CO)OR⁹ and —O(CO)NR⁶R⁷, defined above). Also preferred are compounds wherein R¹ and R² together are ═O.

The sum of q and p is preferably 1 or 2, more preferably 1. Preferred are compounds wherein p is zero and q is 1. More preferred are compounds wherein p is zero, q is 1, Y is —CH₂— and R¹ is —OR⁶, especially when R⁶ is hydrogen.

Another group of preferred compounds is that in which Ar¹ is R³-substituted phenyl, Ar² is R⁴-substituted phenyl and Ar³ is R⁵-substituted phenyl.

Also preferred are compounds wherein Ar¹ is R³-substituted phenyl, Ar² is R⁴-substituted phenyl, Ar³ is R⁵-substituted phenyl, and the sum of p and q is 1 or 2, especially 1. More preferred are compounds wherein Ar¹ is R³-substituted phenyl, Ar² is R⁴-substituted phenyl, Ar³ is R⁵-substituted phenyl, p is zero and q is 1.

A is preferably —O—.

R³ is preferably —COOR⁶, —CONR⁶R⁷, —COR⁶, —SO₂NR⁶R⁷, S(O)₀₋₂-alkyl, S(O)₀₋₂-aryl, NO₂ or halogeno. A more preferred definition for R³ is halogeno, especially fluoro or chloro.

R⁴ is preferably hydrogen, lower alkyl, —OR⁶, —O(CO)R⁶, —O(CO)OR⁹, —O(CO)NR⁶R⁷, —NR⁶R⁷, COR⁶ or halogeno, wherein R⁶ and R⁷ are preferably independently hydrogen or lower alkyl, and R⁹ is preferably lower alkyl. A more preferred definition for R⁴ is hydrogen or halogeno, especially fluoro or chloro.

R⁵ is preferably —OR⁶, —O(CO)R⁶, —O(CO)OR⁹, —O(CO)NR⁶R⁷, —NR⁶R⁷, -(lower alkylene)-COOR⁶ or —CH═CH—COOR⁶, wherein R⁶ and R⁷ are preferably independently hydrogen or lower alkyl, and R⁹ is preferably lower alkyl. A more preferred definition for R⁵ is —OR⁶, -(lower alkylene)-COOR⁶ or —CH═CH—COOR⁶, wherein R⁶ is preferably hydrogen or lower alkyl.

Methods for making compounds of Formula III are well known to those skilled in the art. Non-limiting examples of suitable methods are disclosed in U.S. Pat. No. 5,688,990, which is incorporated herein by reference.

In another embodiment, compounds useful in the methods of the present invention are represented by Formula (IV) below:

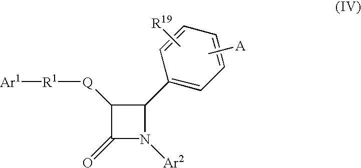

or a pharmaceutically acceptable salt thereof or solvate thereof, wherein, in Formula (IV) above:

A is selected from the group consisting of R²-substituted heterocycloalkyl, R²-substituted heteroaryl, R²-substituted benzofused heterocycloalkyl, and R²-substituted benzofused heteroaryl;

Ar¹ is aryl or R³-substituted aryl;

Ar² is aryl or R⁴-substituted aryl;

Q is a bond or, with the 3-position ring carbon of the azetidinone, forms the spiro group

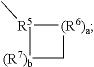

and

R¹ is selected from the group consisting of:

—(CH₂)_{q}—, wherein q is 2-6, provided that when Q forms a spiro ring, q can also be zero or 1;

—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;

—(C₂-C₆ alkenylene)-; and

—(CH₂)_{f}—V—(CH₂)_{g}—, wherein V is C₃-C₆ cycloalkylene, f is 1-5 and g is 0-5, provided that the sum of f and g is 1-6;

R⁵ is selected from:

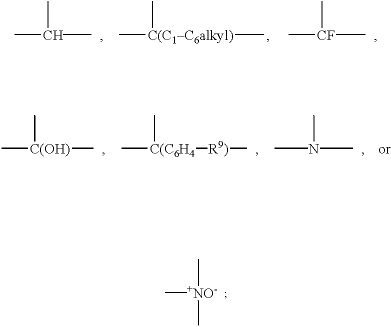

R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)- group;

a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷'s can be the same or different;

and when Q is a bond, R¹ also can be selected from:

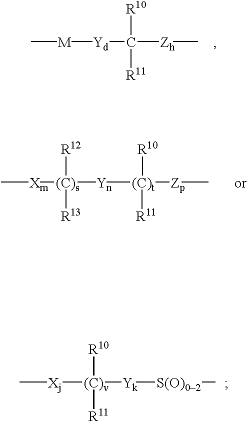

where M is —O—, —S—, —S(O)— or —S(O)₂—;

X, Y and Z are independently selected from the group consisting of —CH₂—, —CH(C₁-C₆ alkyl)- and —C(di-(C₁-C₆) alkyl);

R¹⁰ and R¹² are independently selected from the group consisting of —OR¹⁴, —O(CO)R¹⁴, —O(CO)OR¹⁶ and —O(CO)NR¹⁴R¹⁵;

R¹¹ and R¹³ are independently selected from the group consisting of hydrogen, (C₁-C₆)alkyl and aryl; or R¹⁰ and R¹¹ together are ═O, or R¹² and R¹³ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R¹⁶, —SO₂NR¹⁴R¹⁵ and —(C₁-C₆ alkylene)COOR¹⁴; when R² is a substituent on a heterocycloalkyl ring, R² is as defined, or is ═O or

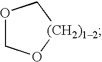

and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸,

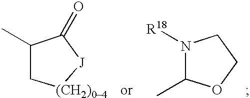

wherein J is —O—, —NH—, —NR¹⁸— or —CH₂—;

R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR¹⁶, —NR¹⁴(CO)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;

R⁸ is hydrogen, (C₁-C₆)alkyl, aryl (C₁-C₆)alkyl, —C(O)R¹⁴ or —COOR¹⁴;

R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;

R¹⁴ and R¹⁵ are independently selected from the group consisting of hydrogen, (C₁-C₆)alkyl, aryl and aryl-substituted (C₁-C₆)alkyl;

R¹⁶ is (C₁-C₆)alkyl, aryl or R¹⁷-substituted aryl;

R¹⁸ is hydrogen or (C₁-C₆)alkyl; and

R¹⁹ is hydrogen, hydroxy or (C₁-C₆)alkoxy.

As used in Formula (IV) above, “A” is preferably an R²-substituted, 6-membered heterocycloalkyl ring containing 1 or 2 nitrogen atoms. Preferred heterocycloalkyl rings are piperidinyl, piperazinyl and morpholinyl groups. The ring “A” is preferably joined to the phenyl ring through a ring nitrogen. Preferred R² substituents are hydrogen and lower alkyl. R¹⁹ is preferably hydrogen.

Ar² is preferably phenyl or R⁴-phenyl, especially (4-R⁴)-substituted phenyl. Preferred definitions of R⁴ are lower alkoxy, especially methoxy, and halogeno, especially fluoro.

Ar¹ is preferably phenyl or R³-substituted phenyl, especially (4-R³)-substituted phenyl.

There are several preferred definitions for the —R¹-Q- combination of variables:

Q is a bond and R¹ is lower alkylene, preferably propylene;

Q is a spiro group as defined above, wherein preferably R⁶ and R⁷ are each ethylene and R⁵ is

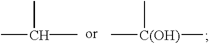

Q is a bond and R¹ is

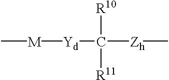

wherein the variables are chosen such that R¹ is —O—CH₂—CH(OH)—;

Q is a bond and R¹ is

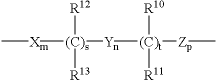

wherein the variables are chosen such that R¹ is —CH(OH)—(CH₂)₂—; and

Q is a bond and R¹ is

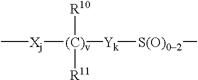

wherein the variables are chosen such that R¹ is —CH(OH)—CH₂—S(O)₀₋₂—.

Methods for making compounds of Formula IV are well known to those skilled in the art. Non-limiting examples of suitable methods are disclosed in U.S. Pat. No. 5,656,624, which is incorporated herein by reference.

In another embodiment, compounds useful in the methods of the present invention are represented by Formula (V) below:

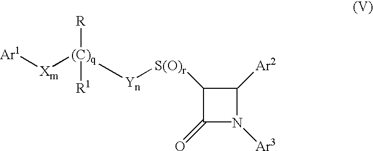

or a pharmaceutically acceptable salt thereof or solvate thereof, wherein, in Formula (V) above:

Ar¹ is aryl, R¹⁰-substituted aryl or heteroaryl;

Ar² is aryl or R⁴-substituted aryl;

Ar³ is aryl or R⁵-substituted aryl;

X and Y are independently selected from the group consisting of —CH₂—, —CH(lower alkyl)- and —C(dilower alkyl)-;

R is —OR⁶, —O(CO)R⁶, —O(CO)OR⁹ or —O(CO)NR⁶R⁷; R¹ is hydrogen, lower alkyl or aryl; or R and R¹ together are ═O;

q is 0 or 1;

r is 0, or 2;

m and n are independently 0, 1, 2, 3, 4 or 5; provided that the sum of m, n and q is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl;

R⁹ is lower alkyl, aryl or aryl-substituted lower alkyl; and

R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, —CF₃, —CN, —NO₂ and halogen.

Within the scope of Formula V, there are included two preferred structures. In Formula VA, q is zero and the remaining variables are as defined above, and in Formula VB, q is 1 and the remaining variables are as defined above:

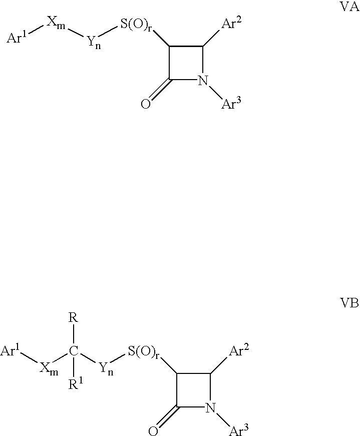

R⁴, R⁵ and R¹⁰ are each preferably 1-3 independently selected substituents as set forth above. Preferred are compounds of Formula (V) wherein Ar¹ is phenyl, R¹⁰-substituted phenyl or thienyl, especially (4-R¹⁰)-substituted phenyl or thienyl. Ar² is preferably R⁴-substituted phenyl, especially (4-R⁴)substituted phenyl. Ar³ is preferably phenyl or R⁵-substituted phenyl, especially (4-R⁵)-substituted phenyl. When Ar¹ is R¹⁰-substituted phenyl, R¹⁰ is preferably halogeno, especially fluoro. When Ar² is R⁴-substituted phenyl, R⁴ is preferably —OR⁶, especially wherein R⁶ is hydrogen or lower alkyl. When Ar³ is R⁵-substituted phenyl, R⁵ is preferably halogeno, especially fluoro. Especially preferred are compounds of Formula (V) wherein Ar¹ is phenyl, 4-fluorophenyl or thienyl, Ar² is 4-(alkoxy or hydroxy)phenyl, and Ar³ is phenyl or 4-fluorophenyl.

X and Y are each preferably —CH₂—. The sum of m, n and q is preferably 2, 3 or 4, more preferably 2. When q is 1, n is preferably 1 to 5.

Preferences for X, Y, Ar¹, Ar² and Ar³ are the same in each of Formulae (VA) and (VB).

In compounds of Formula (VA), the sum of m and n is preferably 2, 3 or 4, more preferably 2. Also preferred are compounds wherein the sum of m and n is 2, and r is 0 or 1.

In compounds of Formula (VB), the sum of m and n is preferably 1, 2 or 3, more preferably 1. Especially preferred are compounds wherein m is zero and n is 1. R¹ is preferably hydrogen and R is preferably —OR⁶ wherein R⁶ is hydrogen, or a group readily metabolizable to a hydroxyl (such as —O(CO)R⁶, —O(CO)OR⁹ and —O(CO)NR⁶R⁷, defined above), or R and R¹ together form a ═O group.

Methods for making compounds of Formula V are well known to those skilled in the art. Non-limiting examples of suitable methods are disclosed in U.S. Pat. No. 5,624,920, which is incorporated herein by reference.

In another embodiment, compounds useful in the methods of the present invention are represented by Formula (VI) below:

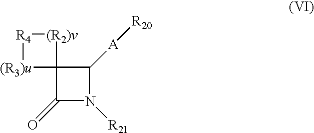

or a pharmaceutically acceptable salt thereof or solvate thereof, wherein in Formula (VI) below:

R₁ is

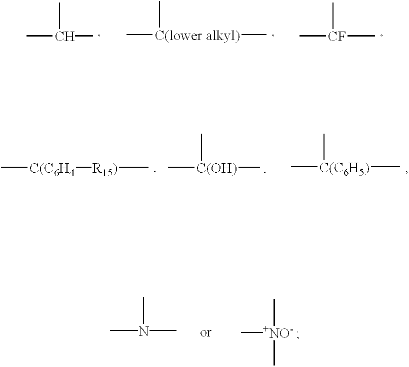

R₂ and R₃ are independently selected from the group consisting of: —CH₂—, —CH(lower alkyl)-, —C(di-lower alkyl)-, —CH═CH— and —C(lower alkyl)=CH—; or R₁ together with an adjacent R₂, or R₁ together with an adjacent R₃, form a —CH═CH— or a —CH═C(lower alkyl)- group;

u and v are independently 0, 1, 2 or 3, provided both are not zero; provided that when R₂ is —CH═CH— or —C(lower alkyl)=CH—, v is 1; provided that when R₃ is —CH═CH— or —C(lower alkyl)=CH—, u is 1; provided that when v is 2 or 3, the R₂'s can be the same or different; and provided that when u is 2 or 3, the R₃'s can be the same or different;

R₄ is selected from B—(CH₂)ₘC(O)—, wherein m is 0, 1, 2, 3, 4 or 5;

B—(CH₂)_{q}—, wherein q is 0, 1, 2, 3, 4, 5 or 6;

B—(CH₂)ₑ-Z-(CH₂)ᵣ—, wherein Z is —O—, —C(O)—, phenylene, —N(R₈)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6;

B—(C₂-C₆ alkenylene)-;

B—(C₄-C₆ alkadienylene)-;

B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6;

B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum off and g is 1, 2, 3, 4, 5 or 6;

B—(CH₂)ₜ—V—(C₂-C₆ alkenylene)- or

B—(C₂-C₆ alkenylene)-V—(CH₂)ₜ—, wherein V and t are as defined above, provided that the sum of t and the number of carbon atoms in the alkenylene chain is 2, 3, 4, 5 or 6;

B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or

T-(CH₂)ₛ—, wherein T is cycloalkyl of 3-6 carbon atoms and s is 0, 1, 2, 3, 4, 5 or 6; or

R₁ and R₄ together form the group

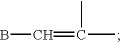

B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or

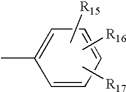

W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R₇-benzyl, benzyloxy, R₇-benzyloxy, phenoxy, R₇-phenoxy, dioxolanyl, NO₂, —N(R₈)(R₉), N(R₈)(R₉)-lower alkylene-, N(R₈)(R₉)-lower alkylenyloxy-, OH, halogeno, —CN, —N₃, —NHC(O)OR₁₀, —NHC(O)R₁₀, R₁₁O₂SNH—, (R₁₁O₂S)₂N—, —S(O)₂NH₂, —S(O)₀₋₂R₈, tert-butyldimethyl-silyloxymethyl, —C(O)R₁₂, —COOR₁₉, —CON(R₈)(R₉), —CH═CHC(O)R₁₂, -lower alkylene-C(O)R₁₂, R₁₀C(O)(lower alkylenyloxy)-, N(R₈)(R₉)C(O)(lower alkylenyloxy)- and

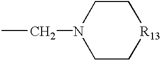

for substitution on ring carbon atoms, and the substituents on the substituted heteroaryl ring nitrogen atoms, when present, are selected from the group consisting of lower alkyl, lower alkoxy, —C(O)OR₁₀, —C(O)R₁₀, OH, N(R₈)(R₉)-lower alkylene-, N(R₈)(R₉)-lower alkylenyloxy-, —S(O)₂NH₂ and 2-(trimethylsilyl)-ethoxymethyl;

R₇ is 1-3 groups independently selected from the group consisting of lower alkyl, lower alkoxy, —COOH, NO₂, —N(R₈)(R₉), OH, and halogeno;

R₈ and R₉ are independently selected from H or lower alkyl;

R₁₀ is selected from lower alkyl, phenyl, R₇-phenyl, benzyl or R₇-benzyl;

R₁₁ is selected from OH, lower alkyl, phenyl, benzyl, R₇-phenyl or R₇-benzyl;

R₁₂ is selected from H, OH, alkoxy, phenoxy, benzyloxy,

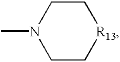

—N(R₈)(R₉), lower alkyl, phenyl or R₇-phenyl;

R₁₃ is selected from —O—, —CH₂—, —NH—, —N(lower alkyl)- or —NC(O)R₁₉;

R₁₅, R₁₆ and R₁₇ are independently selected from the group consisting of H and the groups defined for W; or R₁₅ is hydrogen and R₁₆ and R₁₇, together with adjacent carbon atoms to which they are attached, form a dioxolanyl ring;

R₁₉ is H, lower alkyl, phenyl or phenyl lower alkyl; and

R₂₀ and R₂₁ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above.

One group of preferred compounds of Formula VI is that in which R₂₁ is selected from phenyl, W-substituted phenyl, indanyl, benzofuranyl, benzodioxolyl, tetrahydronaphthyl, pyridyl, pyrazinyl, pyrimidinyl, quinolyl or cyclopropyl,

wherein W is lower alkyl, lower alkoxy, OH, halogeno, —N(R₈)(R₉), —NHC(O)OR₁₀, —NHC(O)R₁₀, NO₂, —CN, —N₃, —SH, —S(O)₀₋₂-(lower alkyl), —COOR₁₉, —CON(R₈)(R₉), —COR₁₂, phenoxy, benzyloxy, —OCF₃, —CH═C(O)R₁₂ or tert-butyldimethylsilyloxy, wherein R₈, R₉, R₁₀, R₁₂ and R₁₉ are as defined for Formula IV. When W is 2 or 3 substituents, the substituents can be the same or different.

Another group of preferred compounds of Formula VI is that in which R₂₀ is phenyl or W-substituted phenyl, wherein preferred meanings of W are as defined above for preferred definitions of R₂₁.

More preferred are compounds of Formula VI wherein R₂₀ is phenyl or W-substituted phenyl and R₂₁ is phenyl, W-substituted phenyl, indanyl, benzofuranyl, benzodioxolyl, tetrahydronaphthyl, pyridyl, pyrazinyl, pyrimidinyl, quinolyl or cyclopropyl; W is lower alkyl, lower alkoxy, OH, halogeno, —N(R₈)(R₉), —NHC(O)OR₁₀, —NHC(O)R₁₀, NO₂, —CN, —N₃, —SH, —S(O)₀₋₂-(lower alkyl), —COOR₁₉, —CON(R₈)(R₉), —COR₁₂, phenoxy, benzyloxy, —CH═CHC(O)R₁₂, —OCF₃ or tert-butyl-dimethyl-silyloxy, wherein when W is 2 or 3 substituents, the substituents can be the same or different, and wherein R₈, R₉, R₁₀, R₁₂ and R₁₉ are as defined in Formula VI.

Also preferred are compounds of Formula VI wherein R₁ is

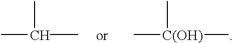

Another group of preferred compounds of Formula VI is in which R₂ and R₃ are each —CH₂— and the sum of u and v is 2, 3 or 4, with u=v=2 being more preferred.

R₄ is preferably B—(CH₂)_{q}— or B—(CH₂)ₑ-Z-(CH₂)ᵣ—, wherein B, Z, q, e and r are as defined above. B is preferably

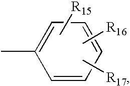

wherein R₁₆ and R₁₇ are each hydrogen and wherein R₁₅ is preferably H, OH, lower alkoxy, especially methoxy, or halogeno, especially chloro.

Preferably Z is —O—, e is 0, and r is 0.

Preferably q is 0-2.

R₂₀ is preferably phenyl or W-substituted phenyl.

Preferred W substituents for R₂₀ are lower alkoxy, especially methoxy and ethoxy, OH, and —C(O)R₁₂, wherein R₁₂ is preferably lower alkoxy.

Preferably R₂₁ is selected from phenyl, lower alkoxy-substituted phenyl and F-phenyl.

Especially preferred are compounds of Formula VI wherein R₁ is

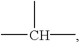

or

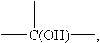

R₂ and R₃ are each —CH₂—, u=v=2, R₄ is B—(CH₂)_{q}—, wherein B is phenyl or phenyl substituted by lower alkoxy or chloro, q is 0-2, R₂₀ is phenyl, OH-phenyl, lower alkoxy-substituted phenyl or lower alkoxycarbonyl-substituted phenyl, and R₂₁ is phenyl, lower alkoxy-substituted phenyl or F-phenyl.

An example of another useful compound of Formula VI is shown below in Formula VIa:

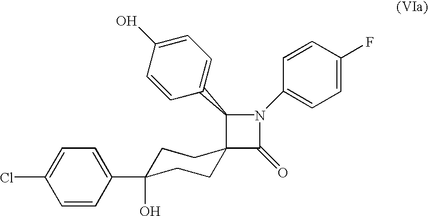

or a pharmaceutically acceptable salt or solvate thereof.

Methods for making compounds of Formula VI are well known to those skilled in the art. Non-limiting examples of suitable methods are disclosed in U.S. Pat. No. 5,698,548, which is incorporated herein by reference.

In another embodiment, compounds useful in the methods of the present invention are represented by Formula (VII):

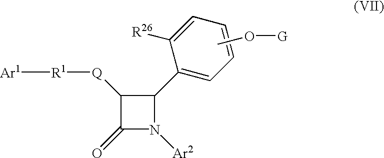

or a pharmaceutically acceptable salt thereof or solvate thereof, wherein, in Formula (VII) above,

R²⁶ is H or OG¹;

G and G¹ are independently selected from the group consisting of

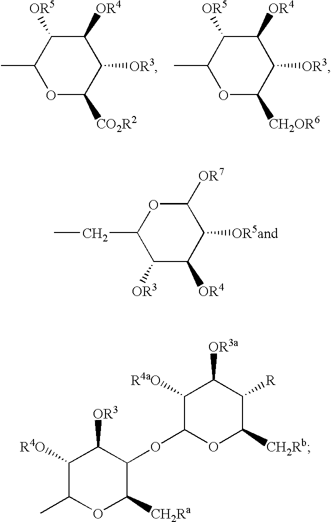

provided that when R²⁶ is H or OH, G is not H;

R, R^{a} and R^{b} are independently selected from the group consisting of H, —OH, halogeno, —NH₂, azido, (C₁-C₆)alkoxy(C₁-C₆)-alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is selected from the group consisting of H and (C₁-C₄)alkyl;

T is selected from the group consisting of phenyl, furyl, thienyl, pyrrolyl, oxazolyl, isoxazolyl, thiazolyl, iosthiazolyl, benzothiazolyl, thiadiazolyt, pyrazolyl, imidazolyl and pyridyl;

R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)-(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is a bond or, with the 3-position ring carbon of the azetidinone, forms the spiro group

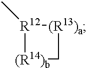

and

R¹ is selected from the group consisting of

—(CH₂)_{q}—, wherein q is 2-6, provided that when Q forms a spiro ring, q can also be zero or 1;

—(CH₂)ₑ-E-(CH₂)ᵣ—, wherein E is —O—, —C(O)—, phenylene, —NR²²— or —S(O)₀₋₂-, e is 0-5 and r is 0-5, provided that the sum of e and r is 1-6;

—(C₂-C₆)alkenylene-; and

—(CH₂)_{f}—V—(CH₂)_{g}—, wherein V is C₃-C₆ cycloalkylene, f is 1-5 and g is 0-5, provided that the sum of f and g is 1-6;

R¹² is

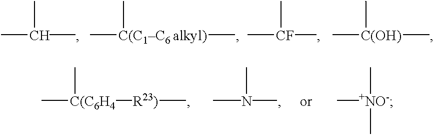

R¹³ and R¹⁴ are independently selected from the group consisting of

—CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero;

provided that when R¹³ is —CH═CH— or —C(C₁-C₆ alkyl)=CH—, a is 1;

provided that when R¹⁴ is —CH═CH— or —C(C₁-C₆ alkyl)=CH—, b is 1;

provided that when a is 2 or 3, the R¹³'s can be the same or different; and

provided that when b is 2 or 3, the R¹⁴'s can be the same or different;

and when Q is a bond, R¹ also can be:

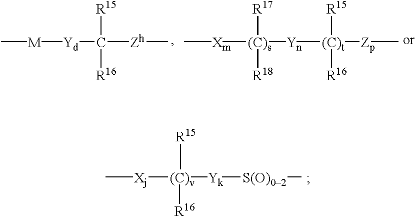

M is —O—, —S—, —S(O)— or —S(O)₂—;

X, Y and Z are independently selected from the group consisting of —CH₂—, —CH(C₁-C₆)alkyl- and —C(di-(C₁-C₆)alkyl);

R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;

R¹⁵ and R¹⁷ are independently selected from the group consisting of —OR¹⁹, —O(CO)R¹⁹, —O(CO)OR²¹ and —O(CO)NR¹⁹R²⁰;

R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;

d is 1, 2 or 3;

h is 0, 1, 2, 3 or 4;

s is 0 or 1; t is 0 or 1; m, n and p are independently 0-4;

provided that at least one of s and t is 1, and the sum of m, n, p, s and t is 1-6;

provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

and when Q is a bond and R¹ is

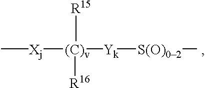

Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —COOR¹⁹;

R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and

R²⁵ is H, —OH or (C₁-C₆)alkoxy.

Ar² is preferably phenyl or R¹¹-phenyl, especially (4-R¹¹)-substituted phenyl. Preferred definitions of R¹¹ are lower alkoxy, especially methoxy, and halogeno, especially fluoro.

Ar¹ is preferably phenyl or R¹⁰-substituted phenyl, especially (4-R¹⁰)-substituted phenyl. Preferably R¹⁰ is halogeno, and more preferably fluoro.

There are several preferred definitions for the —R¹-Q- combination of variables:

Q is a bond and R¹ is lower alkylene, preferably propylene;

Q is a spiro group as defined above, wherein preferably R¹³ and R¹⁴ are each ethylene and R¹² is

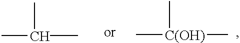

and R¹ is —(CH₂)_{q} wherein q is 0-6;

Q is a bond and R¹ is

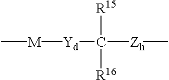

wherein the variables are chosen such that R¹ is —O—CH₂—CH(OH)—;

Q is a bond and R¹

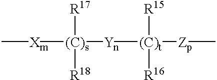

wherein the variables are chosen such that R¹ is —CH(OH)—(CH₂)₂—; and

Q is a bond and R¹ is

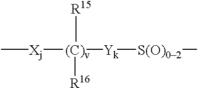

wherein the variables are chosen such that R¹ is —CH(OH)—CH₂—S(O)₀₋₂—.

A preferred compound of Formula (VII) therefore, is one wherein G and G¹ are as defined above and in which the remaining variables have the following definitions:

Ar¹ is phenyl or R¹⁰-substituted phenyl, wherein R¹⁰ is halogeno;

Ar² is phenyl or R¹¹-phenyl, wherein R¹¹ is 1 to 3 substituents independently selected from the group consisting of C₁-C₆ alkoxy and halogeno;

Q is a bond and R¹ is lower alkylene; Q, with the 3-position ring carbon of the azetidinone, forms the group

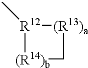

wherein preferably R¹³ and R¹⁴ are each ethylene and a and b are each 1, and wherein R¹² is

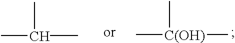

Q is a bond and R¹ is —O—CH₂—CH(OH)—; Q is a bond and R¹ is —CH(OH)—(CH₂)₂—; or Q is a bond and R¹ is —CH(OH)—CH₂—S(O)₀₋₂—.

Preferred variables for G and G¹ groups of the formulae

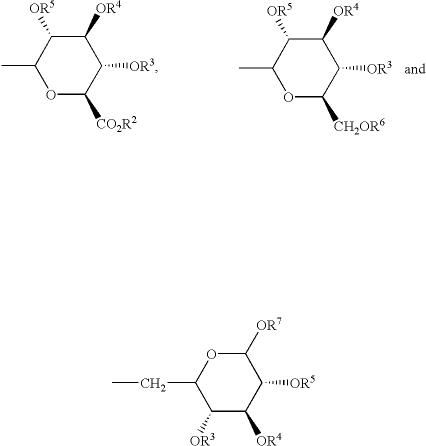

are as follows:

R², R³, R⁴, R⁵, R⁶ and R⁷ are independently selected from the group consisting of H, (C₁-C₆)alkyl, benzyl and acetyl.

Preferred variables for group G or G¹ of the formula:

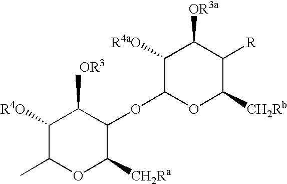

are as follows:

R³, R^{3a}, R⁴ and R^{4a} are selected from the group consisting of H, (C₁-C₆)alkyl, benzyl and acetyl;

R, R^{a} and R^{b} are independently selected from the group consisting of H, —OH, halogeno, —NH₂, azido, (C₁-C₆)alkoxy(C₁-C₆)alkoxy and —W—R³⁰,

wherein W is —O—C(O)— or —O—C(O)—NR³¹—, R³¹ is H and R³⁰ is (C₁-C₆)alkyl, —C(O)—(C₁-C₄)alkoxy-(C₁-C₆)alkyl, T, T-(C₁-C₆)alkyl, or T or T-(C₁-C₆)alkyl wherein T is substituted by one or two halogeno or (C₁-C₆)alkyl groups.

Preferred R³⁰ substituents are selected from the group consisting of: 2-fluorophenyl, 2,4-difluoro-phenyl, 2,6-dichlorophenyl, 2-methylphenyl, 2-thienylmethyl, 2-methoxy-carbonylethyl, thiazol-2-yl-methyl, 2-furyl, 2-methoxycarbonylbutyl and phenyl.

Preferred combinations of R, R^{a} and R^{b} are as follows:

1) R, R^{a} and R^{b} are independently —OH or —O—C(O)—NH—R³⁰, especially wherein R^{a} is —OH and R and R^{b} are —O—C(O)—NH—R³⁰ and R³⁰ is selected from the preferred substituents identified above, or wherein R and R^{a} are each —OH and R^{b} is —O—C(O)—NH—R³⁰ wherein R³⁰ is 2-fluorophenyl, 2,4-difluoro-phenyl, 2,6-dichlorophenyl;

2) R^{a} is —OH, halogeno, azido or (C₁-C₆)-alkoxy(C₁-C₆)alkoxy, R^{b} is H, halogeno, azido or (C₁-C₆)alkoxy(C₁-C₆)-alkoxy, and R is —O—C(O)—NH—R³⁰, especially compounds wherein R^{a} is —OH, R^{b} is H and R³⁰ is 2-fluorophenyl;

3) R, R^{a} and R^{b} are independently —OH or —O—C(O)—R³⁰ and R³⁰ is (C₁-C₆)alkyl, T, or T substituted by one or two halogeno or (C₁-C₆)alkyl groups, especially compounds wherein R is —OH and R^{a} and R^{b} are —O—C(O)—R³⁰ wherein R³⁰ is 2-furyl; and

4) R, R^{a} and R^{b} are independently —OH or halogeno. Three additional classes of preferred compounds are those wherein the C^{1′} anomeric oxy is beta, wherein the C^{2′} anomeric oxy is beta, and wherein the R group is alpha. G and G¹ are preferably selected from:

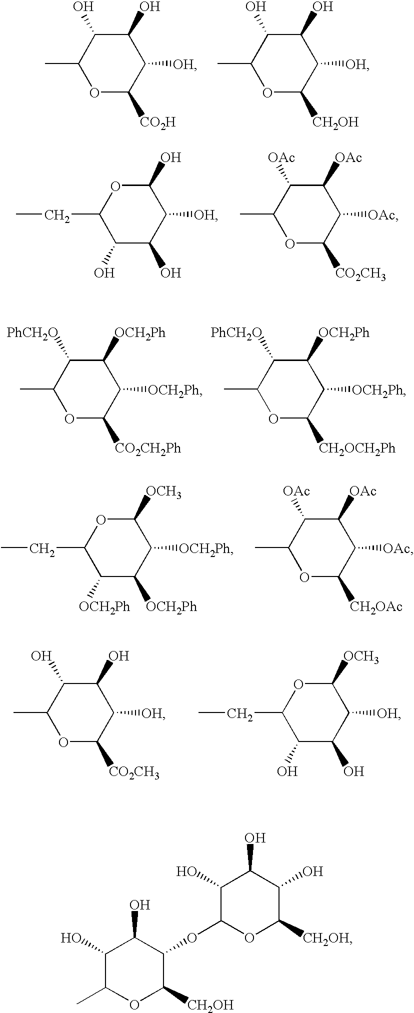

wherein Ac is acetyl and Ph is phenyl.

Preferably, R²⁶ is H or OH, more preferably H. The -O-G substituent is preferably in the 4-position of the phenyl ring to which it is attached.

In another embodiment, compounds useful in the methods of the present invention are represented by Formula (VIII) below:

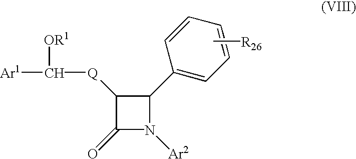

or a pharmaceutically acceptable salt or solvate thereof, wherein in Formula (VIII) above:

R²⁶ is selected from the group consisting of:

a) OH;

b) OCH₃;

c) fluorine and

d) chlorine.

R¹ is selected from the group consisting of

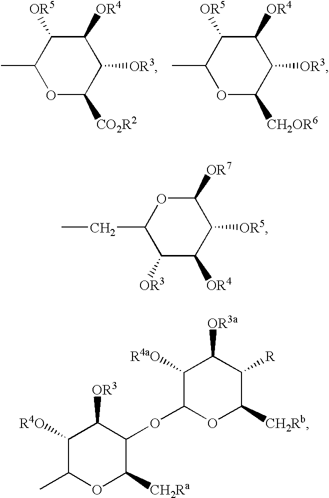

—SO₃H; natural and unnatural amino acids.

R, R^{a} and R^{b} are independently selected from the group consisting of H, —OH, halogeno, —NH₂, azido, (C₁-C₆)alkoxy(C₁-C₆)-alkoxy and —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is independently selected form the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is independently selected from the group consisting of H and (C₁-C₄)alkyl;

T is independently selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of H, halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is —(CH₂)_{q}—, wherein q is 2-6, or, with the 3-position ring carbon of the azetidinone,

forms the spiro group

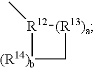

R¹² is

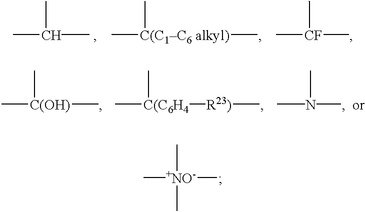

R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, the R¹³'s can be the same or different; and provided that when b is 2 or 3, the R¹⁴'s can be the same or different;

R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;

Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —COOR¹⁹;

R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and

R²⁵ is H, —OH or (C₁-C₆)alkoxy.

Ar² is preferably phenyl or R¹¹-phenyl, especially (4-R¹¹)-substituted phenyl. Preferred definitions of R¹¹ are lower alkoxy, especially methoxy, and halogeno, especially fluoro.

Ar¹ is preferably phenyl or R¹⁰-substituted phenyl, especially (4-R¹⁰)-substituted phenyl. A preferred definition of R¹⁰ is halogeno, especially fluoro.

Preferably Q is a lower alkyl or a spiro group as defined above, wherein preferably R¹³ and R¹⁴ are each ethylene and R¹² is

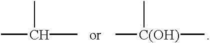

A preferred compound of formula VIII, therefore, is one wherein R¹ is as defined above and in which the remaining variables have the following definitions:

Ar¹ is phenyl or R¹⁰-substituted phenyl, wherein R¹⁰ is halogeno;

Ar² is phenyl or R¹¹-phenyl, wherein R¹¹ is 1 to 3 substituents independently selected from the group consisting of C₁-C₆ alkoxy and halogeno;

Q is a lower alkyl (i.e. C-1 to C-2) with Q=C-2 being preferred, or Q, with the 3-position ring carbon of the azetidinone, forms the group

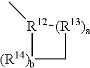

wherein preferably R¹³ and R¹⁴ are each ethylene and a and b are each 1, and wherein R¹² is

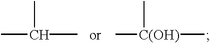

Preferred variables for R¹ groups of the formula

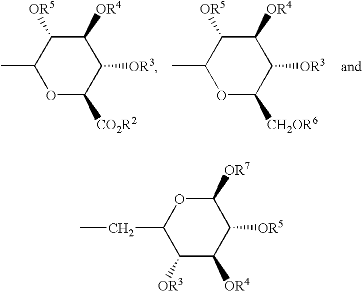

are as follows:

R², R³, R⁴, R⁵, R⁶ and R⁷ are independently selected from the group consisting of H, (C₁-C₆)alkyl, benzyl and acetyl.

Preferred variables for group R¹ of the formula

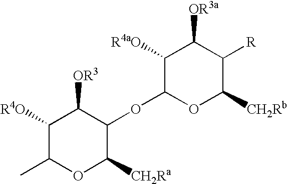

are as follows:

R³, R^{3a}, R⁴ and R₄ₐ are selected from the group consisting of H, (C₁-C₆)alkyl, benzyl and acetyl;

R, R^{a} and R^{b} are independently selected from the group consisting of H, —OH, halogeno, —NH₂, azido, (C₁-C₆)alkoxy(C₁-C₆)alkoxy and —W—R³⁰, wherein W is —O—C(O)— or —O—C(O)—NR³¹—, R³¹ is H and R³⁰ is (C₁-C₆)alkyl, —C(O)—(C₁-C₄)alkoxy-(C₁-C₆)alkyl, T , T-(C₁-C₆)alkyl, or T or T-(C₁-C₆)alkyl wherein T is substituted by one or two halogeno or (C₁-C₆)alkyl groups.

Preferred R³⁰ substituents are 2-fluorophenyl, 2,4-difluoro-phenyl, 2,6-dichlorophenyl, 2-methylphenyl, 2-thienylmethyl, 2-methoxy-carbonylethyl, thiazol-2-yl-methyl, 2-furyl, 2-methoxycarbonylbutyl and phenyl. Preferred combinations of R, R^{a} and R^{b} are as follows: 1) R, R^{a} and R^{b} are independently —OH or —O—C(O)—NH—R³⁰, especially wherein R^{a} is —OH and R and R^{b} are —O—C(O)—NH—R³⁰ and R³⁰ is selected from the preferred substituents identified above, or wherein R and R^{a} are —OH and R^{b} is —O—C(O)—NH—R³⁰ wherein R³⁰ is 2-fluorophenyl, 2,4-difluoro-phenyl, 2,6-dichlorophenyl; 2) R^{a} is —OH, halogeno, azido or (C₁-C₆)-alkoxy(C₁-C₆)alkoxy, R^{b} is H, halogeno, azido or (C₁-C₆)alkoxy(C₁-C₆)-alkoxy, and R is —O—C(O)—NH—R³⁰, especially compounds wherein R^{a} is —OH, R^{b} is H and R³⁰ is 2-fluorophenyl; 3) R, R^{a} and R^{b} are independently —OH or —O—C(O)—R³⁰ and R³⁰ is (C₁-C₆)alkyl, T, or T substituted by one or two halogeno or (C₁-C₆)alkyl groups, especially compounds wherein R is —OH and R^{a} and R^{b} are —O—C(O)—R³⁰ wherein R³⁰ is 2-furyl; and 4) R, R^{a} and R^{b} are independently —OH or halogeno. Three additional classes of preferred are compounds are those wherein the C^{1′} anomeric oxy is beta, wherein the C^{2′} anomeric oxy is beta, and wherein the R group is alpha.

R¹ is preferably selected from:

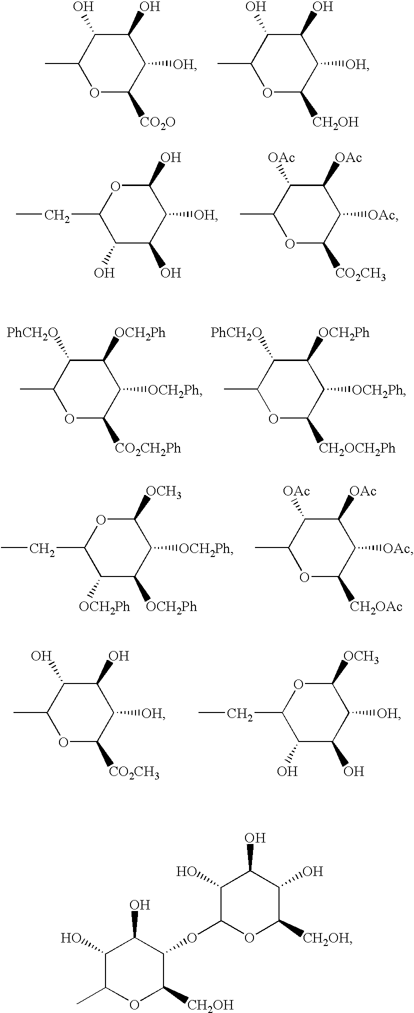

wherein Ac is acetyl and Ph is phenyl.

An example of a useful compound of this invention is one represented by the formula IX:

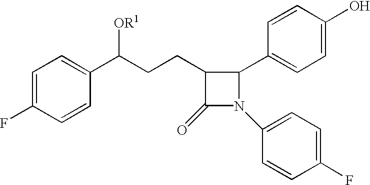

wherein R¹ is defined as above, or a pharmaceutically acceptable salt or solvate thereof.

A more preferred compound is one represented by formula X:

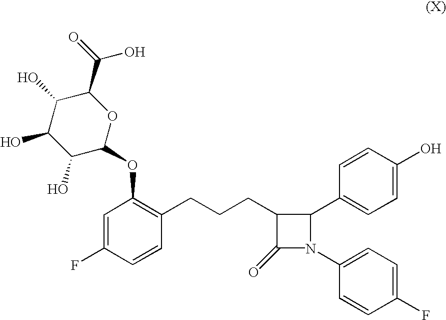

or a pharmaceutically acceptable salt or solvate thereof.

The compounds of Formulae I-X can be prepared by known methods, including the methods discussed above and, for example, WO 93/02048 describes the preparation of compounds wherein —R¹-Q- is alkylene, alkenylene or alkylene interrupted by a hetero atom, phenylene or cycloalkylene; WO 94/17038 describes the preparation of compounds wherein Q is a spirocyclic group; WO 95/08532 describes the preparation of compounds wherein —R¹-Q- is a hydroxy-substituted alkylene group; PCT/US95/03196 describes compounds wherein —R¹-Q- is a hydroxy-substituted alkylene attached to the Ar¹ moiety through an —O— or S(O)₀₋₂- group; and U.S. Ser. No. 08/463,619, filed Jun. 5, 1995, describes the preparation of compounds wherein —R¹-Q- is a hydroxy-substituted alkylene group attached the azetidinone ring by a —S(O)₀₋₂— group.

The daily dose of the compound of Formula I-X administered to the subject can range from about 0.1 to about 1000 mg per day, preferably about 0.25 to about 50 mg/day, and more preferably about 10 mg per day, given in a single dose or 2-4 divided doses. The exact dose, however, is determined by the attending clinician and is dependent on the potency of the compound administered, the age, weight, condition and response of the subject.

For administration of pharmaceutically acceptable salts of the above compounds, the weights indicated above refer to the weight of the acid equivalent or the base equivalent of the therapeutic compound derived from the salt.

In another alternative embodiment, the compositions used in the methods of the present invention can further comprise one or more Alzheimer's treatments different from compounds I-X discussed above (e.g., different in chemical structure) in combination with one or more of compounds I-X above.

Non-limiting examples of suitable treatments which can be useful in treating Alzheimer's Disease include administration of one or more of the following: cholinesterase inhibitors, muscarinic receptor agonists, M2 muscarinic receptor antagonists, acetylcholine release stimulators, choline uptake stimulators, nicotinic cholinergic receptor agonists, anti-Aβ vaccines, γ-secretase inhibitors, β-secretase inhibitors, amyloid aggregation inhibitors, amyloid precursor protein antisense oligonucleotides, monoamine reuptake inhibitors, human stem cells, gene therapy, nootropic agents, AMPA receptor ligands, growth factors or growth factor receptor agonists, anti-inflammatory agents, free radical scavengers, antioxidants, superoxide dismutase stimulators, calcium channel blockers, apoptosis inhibitors, caspase inhibitors, monoamine oxidase inhibitors, estrogens and estrogen receptor ligands, NMDA receptor antagonists, Jun N-terminal kinase (JNK) inhibitors, copper/zinc chelators, 5-HT1a receptor agonists, NGF stimulators, neuroprotective agents, H3 histamine receptor antagonists, calpain inhibitors, poly ADP ribose polymerase inhibitors, prolylendopeptidase inhibitors, calcium modulators, corticortropin releasing factor receptor antagonists, corticortropin releasing factor binding protein inhibitors, GABA modulators, GABA-A receptor antagonists, GABA-B receptor antagonists, neuroimmunophilin ligands, sigma receptor ligands, galanin receptor ligands, imidazoline/alpha adrenergic receptor antagonists, vasoactive intestinal peptide receptor agonists, benzodiazepine receptor inverse agonists, cannabinoid receptor agonists, thyrotropin releasing hormone receptor agonists, protein kinase C inhibitors, 5-HT3 receptor antagonists, prostaglandin receptor antagonists, topoisomerase II inhibitors, steroid receptor ligand, nitric oxide modulators, RAGE inhibitors, dopamine receptor agonists, and combinations thereof.

Suitable cholinesterase inhibitors include donepezil hydrochloride (such as ARICEPT which is available from Pfizer), rivastigmine tartrate (such as EXELON which is available from Novartis), tacrine (such as COGNEX which is available from Parke-Davis), galanthamine derivatives available from Janssen, metrifonate available from Bayer Corp., ipidacrine available from Nikken Chemicals Co. Ltd., TAK-147, T-82 available from SS Pharmaceutical Co. Ltd., methanesulfonyl fluoride, CHF-2819, phenserine, physostigmine available from Forest Laboratories, Inc., huperzine, cymserine available from Anonyx Inc., tolserine available from National Institutes of Health, ER-127528 available from Eisai Co. Ltd., and combinations thereof.

Useful muscarinic receptor agonists include cevimeline, PD-151832 available from Pfizer Inc., YM-796 available from Yamanouchi Pharmaceutical Inc., P-58 available from Phytopharm plc and combinations thereof.

Suitable acetylcholine release stimulators include minaprine, montirelin available from Grunenthal GmbH, T-588 available from Toyama Chemical Co. Ltd., XE-991 and combinations thereof. Useful choline uptake stimulators include MKC-231 available from Mitsubishi-Tokyo Pharmaceuticals Inc.

Suitable nicotinic cholinergic receptor agonists include altinicline available from SIBIA Neurosciences Inc., SIB-1553A, ABT-089 (disclosed in U.S. Pat. No. 5,278,176 and available from Abbott Laboratories), nicotine patch, GRS-21, TC-2403 and combinations thereof.

Suitable anti-Aβ vaccines include AN-1792.

Suitable amyloid aggregation inhibitors include reumacon available from Conpharm AB, NC-531 available from Neurochem Inc., PPI-1019 available from Praecis Pharmaceuticals Inc. and combinations thereof.

Suitable monoamine reuptake inhibitors include NS-2330.

Suitable nootropic agents include oxiracetam available from ISF Societa Per Azioni, pramiracetam available from Warner Lambert Co., idebenone available from Takeda Chemical Inds. Ltd., anapsos available from ASAC Pharmaceuticals International, nebracetam available from Boehringer Ingelheim Corp., JTP-2942 available from Japan Tobacco Inc., fasoracetam available from Nippon Shinyaku Co. Ltd., bacosides available from Central Drug Research Institute, alzene available from Bar-IIan University, KA-672 available from Dr. Willmar Schwabe GmbH & Co., alaptid available from VUFB, IQ-200, ALE-26015 available from Allelix Pharm-Eco LP and combinations thereof.

Useful AMPA receptor ligands include CX-516, CX-691 available from Cortex Pharmaceuticals Inc. and combinations thereof.

Suitable growth factors or growth factor receptor agonists include leteprinim.

Suitable anti-inflammatory agents include COX2 inhibitors (such as VIOXX rofecoxib available from Merck & Co., Inc. and CELEBREX celecoxib available from Pfizer), cytokine inhibitors (such as thalidomide disclosed in WO 95/04533 and dexanabinol) complement inhibitors, leukotriene receptor antagonists and combinations thereof.

Useful free radical scavengers/antioxidants include EGb-761 available from Yuyu Industrial Co., CPI-22, dexanabinol and combinations thereof.

Suitable calcium channel blockers include tamolarizine available from Nippon Chemiphar Co., Ltd., nimodipine available from Bayer AG, PD-1 76078 available from Elan Pharmaceuticals, Inc., and combinations thereof.

Suitable apoptosis inhibitors include acetyl-L-carnitine, CEP-1347 available from Cephalon, Inc., TCH-346 available from Novartis AG and combinations thereof.

A useful caspase inhibitor is pralnacasan.

Suitable monoamine oxidase inhibitors include moclobemide available from Roche Holding AG, selegiline, rasagiline available from Teva Pharmaceutical Inds. Ltd., SL-25.1188, Ro-41-1049 available from Roche Holding AG, and combinations thereof.

Suitable NMDA receptor antagonists include memantine, ipenoxazone available from Nippon Chemiphar Co. Ltd. and combinations thereof.

Suitable copper/zinc chelators include clioquinol available from PN Gerolymatos SA.

A useful 5-HT1a receptor agonist is AP-159 available from Asahi Kasei Corp.; a suitable NGF stimulator is xaliprodene available from Sanofi-Synthelabo.

Suitable neuroprotective agents include citicholine, GS-1590 available from Leo Pharmaceutical Products Ltd. A/S, CPI-1189 available from Centaur Pharmaceuticals Inc., SR-57667 available from Sanofi-Synthelabo and combinations thereof.

Suitable H3 histamine receptor antagonists include GT-2016 and GT-2331 (both available from Gliatech, Inc.) and combinations thereof.

Useful prolylendopeptidase inhibitors include ONO-1603 available from Ono Pharmaceutical Co. Ltd., Z-321 available from Zeria Pharmaceutical Co. Ltd. and combinations thereof.

A useful calcium modulator includes neurocalc available from Apollo Biopharmaceuticals Inc.

A suitable corticortropin releasing factor receptor antagonist includes NBI-113 available from Neurocrine Biosciences, Inc.

A useful GABA modulator includes NGD 97-1 available from Neurogen Corp.

A suitable sigma receptor ligand is igmesine available from Pfizer Inc.

A useful imidazoline/alpha adrenergic receptor antagonist is efaroxan available from Reckitt & Colman PLC.

A suitable vasoactive intestinal peptide receptor agonist is stearyl-NIe-VIP.

A useful benzodiazepine inverse agonist is S-8510 available from Shionogi & Co. Ltd.

A suitable cannabinoid receptor agonist is dronabinol available from Unimed Pharmaceuticals Inc.

Useful thyrotropin releasing hormone receptor agonists include taltireline available from Tanabe Seiyaku Co. Ltd. and protirelin available from Takeda Chemical Inds., Inc.

A suitable 5-HT3 antagonist is GYKI-46903.

A useful topoisomerase II inhibitor is iododoxorubicin available from Pharmacia & Upjohn AB.

A suitable steroid receptor agonist is GL-701 available from Leland Stanford Junior University.

A useful corticosteroid receptor antagonist is anticort.

A suitable nitric oxide modulator is GL-701.

A suitable RAGE inhibitor is ALT-711 available from Alteon Inc.

A useful dopamine receptor agonist is speramine.

Generally, a total daily dosage of Alzheimer's treatment(s) different from compounds I-X as discussed above can range from about 0.01 to about 1000 mg/day, and preferably about 0.5 to about 20 mg/kg body weight/day in single or divided doses.

In another embodiment of the present invention, the compositions used in the methods of the present invention can further comprise one or more pharmacological or therapeutic agents or drugs such as cholesterol biosynthesis inhibitors and/or other lipid-lowering agents, as discussed below.

Non-limiting examples of suitable cholesterol biosynthesis inhibitors include competitive inhibitors of HMG CoA reductase, the rate-limiting step in cholesterol biosynthesis, squalene synthase inhibitors, squalene epoxidase inhibitors and mixtures thereof. Non-limiting examples of suitable HMG CoA reductase inhibitors include statins such as lovastatin (for example MEVACOR® which is available from Merck & Co.), pravastatin (for example PRAVACHOL® which is available from Bristol Meyers Squibb), fluvastatin, simvastatin (for example ZOCOR® which is available from Merck & Co.), atorvastatin, cerivastatin, CI-981 and pitavastatin (such as NK-104 of Negma Kowa of Japan), rosuvastatin; HMG CoA synthetase inhibitors, for example L-659,699 ((E,E)-11-[3′R-(hydroxy-methyl)-4′-oxo-2′R-oxetanyl]-3,5,7R-trimethyl-2,4-undecadienoic acid); squalene synthesis inhibitors, for example squalestatin 1; and squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride) and other sterol biosynthesis inhibitors such as DMP-565. Preferred HMG CoA reductase inhibitors include lovastatin, pravastatin and simvastatin. The most preferred HMG CoA reductase inhibitor is simvastatin.

Generally, a total daily dosage of cholesterol biosynthesis inhibitor(s) can range from about 0.1 to about 160 mg per day, and preferably about 0.2 to about 80 mg/day in single or 2-3 divided doses.

In another embodiment, the compositions used in the compositions and methods of the present invention can further comprise one or more other lipid lowering agents as discussed below. For example, one or more PPAR activators, can be coadministered with or in combination with the compound(s) of Formulae I-X discussed above.

Also useful in the present invention are compositions or therapeutic combinations that further comprise at least one (one or more) activators for peroxisome proliferator-activated receptors (PPAR). The activators act as agonists for the peroxisome proliferator-activated receptors. Three subtypes of PPAR have been identified, and these are designated as peroxisome proliferator-activated receptor alpha (PPARα), peroxisome proliferator-activated receptor gamma (PPARγ) and peroxisome proliferator-activated receptor delta (PPARδ). It should be noted that PPARδ is also referred to in the literature as PPARβ and as NUC1, and each of these names refers to the same receptor.

PPARα regulates the metabolism of lipids. PPARα is activated by fibrates and a number of medium and long-chain fatty acids, and it is involved in stimulating β-oxidation of fatty acids. The PPARγ receptor subtypes are involved in activating the program of adipocyte differentiation and are not involved in stimulating peroxisome proliferation in the liver. PPARδ has been identified as being useful in increasing high density lipoprotein (HDL) levels in humans. See, e.g., WO 97/28149.

PPARα activator compounds are useful for, among other things, lowering triglycerides, moderately lowering LDL levels and increasing HDL levels. Useful examples of PPARα activators include fibrates.

Non-limiting examples of suitable fibric acid derivatives (“fibrates”) include clofibrate (such as ethyl 2-(p-chlorophenoxy)-2-methyl-propionate, for example ATROMID-S® Capsules which are commercially available from Wyeth-Ayerst); gemfibrozil (such as 5-(2,5-dimethylphenoxy)-2,2-dimethylpentanoic acid, for example LOPID® tablets which are commercially available from Parke Davis); ciprofibrate (C.A.S. Registry No. 52214-84-3, see U.S. Pat. No. 3,948,973 which is incorporated herein by reference); bezafibrate (C.A.S. Registry No. 41859-67-0, see U.S. Pat. No. 3,781,328 which is incorporated herein by reference); clinofibrate (C.A.S. Registry No. 30299-08-2, see U.S. Pat. No. 3,716,583 which is incorporated herein by reference); binifibrate (C.A.S. Registry No. 69047-39-8, see BE 884722 which is incorporated herein by reference); lifibrol (C.A.S. Registry No. 96609-16-4); fenofibrate (such as TRICOR® micronized fenofibrate (2-[4-(4-chlorobenzoyl) phenoxy]-2-methyl-propanoic acid, 1-methylethyl ester) which is commercially available from Abbott Laboratories or LIPANTHYL® micronized fenofibrate which is commercially available from Labortoire Founier, France) and mixtures thereof. These compounds can be used in a variety of forms, including but not limited to acid form, salt form, racemates, enantiomers, zwitterions and tautomers.

Other examples of PPARα activators useful in the practice of the present invention include suitable fluorophenyl compounds as disclosed in U.S. Pat. No. 6,028,109 which is incorporated herein by reference; certain substituted phenylpropionic compounds as disclosed in WO 00/75103 which is incorporated herein by reference; and PPARα activator compounds as disclosed in WO 98/43081 which is incorporated herein by reference. Non-limiting examples of suitable PPARγ activators include derivatives of glitazones or thiazolidinediones, such as, troglitazone (such as REZULIN® troglitazone (-5-[[4-[3,4-dihydro-6-hydroxy-2,5,7,8-tetramethyl-2H-1-benzopyran-2-yl)methoxy]phenyl]methyl]-2,4-thiazolidinedione) commercially available from Parke-Davis); rosiglitazone (such as AVANDIA® rosiglitazone maleate (-5-[[4-[2-(methyl-2-pyridinylamino)ethoxy]phenyl]methyl]-2,4-thiazolidinedione, (Z)-2-butenedioate) (1:1) commercially available from SmithKline Beecham) and pioglitazone (such as ACTOS™ pioglitazone hydrochloride (5-[[4-[2-(5-ethyl-2-pyridinyl)ethoxy]phenyl]methyl]-2,4-]thiazolidinedione monohydrochloride) commercially available from Takeda Pharmaceuticals). Other useful thiazolidinediones include ciglitazone, englitazone, darglitazone and BRL 49653 as disclosed in WO 98/05331 which is incorporated herein by reference; PPARγ activator compounds disclosed in WO 00/76488 which is incorporated herein by reference; and PPARy activator compounds disclosed in U.S. Pat. No. 5,994,554 which is incorporated herein by reference.

Other useful PPARγ activator compounds include certain acetylphenols as disclosed in U.S. Pat. No. 5,859,051 which is incorporated herein by reference; certain quinoline phenyl compounds as disclosed in WO 99/20275 which is incorporated herein by reference; aryl compounds as disclosed by WO 99/38845 which is incorporated herein by reference; certain 1,4-disubstituted phenyl compounds as disclosed in WO 00/63161; certain aryl compounds as disclosed in WO 01/00579 which is incorporated herein by reference; benzoic acid compounds as disclosed in WO 01/12612 & WO 01/12187 which are incorporated herein by reference; and substituted 4-hydroxy-phenylalconic acid compounds as disclosed in WO 97/31907 which is incorporated herein by reference.

PPARδ compounds are useful for, among other things, lowering triglyceride levels or raising HDL levels. Non-limiting examples of PPARδ activators include suitable thiazole and oxazole derivatives, such as C.A.S. Registry No. 317318-32-4, as disclosed in WO 01/00603 which is incorporated herein by reference); certain fluoro, chloro or thio phenoxy phenylacetic acids as disclosed in WO 97/28149 which is incorporated herein by reference; suitable non-β-oxidizable fatty acid analogues as disclosed in U.S. Pat. No. 5,093,365 which is incorporated herein by reference; and PPARδ compounds as disclosed in WO 99/04815 which is incorporated herein by reference.

Moreover, compounds that have multiple functionality for activating various combinations of PPARα, PPARγ and PPARδ are also useful with the practice of the present invention. Non-limiting examples include certain substituted aryl compounds as disclosed in U.S. Pat. No. 6,248,781; WO 00/23416; WO 00/23415; WO 00/23425; WO 00/23445; WO 00/23451; and WO 00/63153, all of which are incorporated herein by reference, are described as being useful PPARα and/or PPARγ activator compounds. Other non-limiting examples of useful PPARα and/or PPARγ activator compounds include activator compounds as disclosed in WO 97/25042 which is incorporated herein by reference; activator compounds as disclosed in WO 00/63190 which is incorporated herein by reference; activator compounds as disclosed in WO 01/21181 which is incorporated herein by reference; biaryl-oxa(thia)zole compounds as disclosed in WO 01/16120 which is incorporated herein by reference; compounds as disclosed in WO 00/63196 and WO 00/63209 which are incorporated herein by reference; substituted 5-aryl-2,4-thiazolidinediones compounds as disclosed in U.S. Pat. No. 6,008,237 which is incorporated herein by reference; arylthiazolidinedione and aryloxazolidinedione compounds as disclosed in WO 00/78312 and WO 00/78313G which are incorporated herein by reference; GW2331 or (2-(4-[difluorophenyl]-1heptylureido)ethyl]phenoxy)-2-methylbutyric compounds as disclosed in WO 98/05331 which is incorporated herein by reference; aryl compounds as disclosed in U.S. Pat. No. 6,166,049 which is incorporated herein by reference; oxazole compounds as disclosed in WO 01/17994 which is incorporated herein by reference; and dithiolane compounds as disclosed in WO 01/25225 and WO 01/25226 which are incorporated herein by reference.

Other useful PPAR activator compounds include substituted benzylthiazolidine-2,4-dione compounds as disclosed in WO 01/14349, WO 01/14350 and WO/01/04351 which are incorporated herein by reference; mercaptocarboxylic compounds as disclosed in WO 00/50392 which is incorporated herein by reference; ascofuranone compounds as disclosed in WO 00/53563 which is incorporated herein by reference; carboxylic compounds as disclosed in WO 99/46232 which is incorporated herein by reference; compounds as disclosed in WO 99/12534 which is incorporated herein by reference; benzene compounds as disclosed in WO 99/15520 which is incorporated herein by reference; o-anisamide compounds as disclosed in WO 01/21578 which is incorporated herein by reference; and PPAR activator compounds as disclosed in WO 01/40192 which is incorporated herein by reference.

The peroxisome proliferator-activated receptor(s) activator(s) are administered in a therapeutically effective amount to treat the specified condition, for example in a daily dose preferably ranging from about 50 to about 3000 mg per day, and more preferably about 50 to about 2000 mg per day, given in a single dose or 2-4 divided doses. The exact dose, however, is determined by the attending clinician and is dependent on such factors as the potency of the compound administered, the age, weight, condition and response of the subject.

In another alternative embodiment, the compositions used in the methods of the present invention can further comprise one or more bile acid sequestrants (insoluble anion exchange resins), coadministered with or in combination with the compound(s) of Formulae I-X discussed above.

Bile acid sequestrants bind bile acids in the intestine, interrupting the enterohepatic circulation of bile acids and causing an increase in the faecal excretion of steroids. Use of bile acid sequestrants is desirable because of their non-systemic mode of action. Bile acid sequestrants can lower intrahepatic cholesterol and promote the synthesis of apo B/E (LDL) receptors which bind LDL from plasma to further reduce cholesterol levels in the blood.

Non-limiting examples of suitable bile acid sequestrants include cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof. Other useful bile acid sequestrants are disclosed in PCT Patent Applications Nos. WO 97/11345 and WO 98/57652, and U.S. Pat. Nos. 3,692,895 and 5,703,188 which are incorporated herein by reference. Suitable inorganic cholesterol sequestrants include bismuth salicylate plus montmorillonite clay, aluminum hydroxide and calcium carbonate antacids.

Generally, a total daily dosage of bile acid sequestrant(s) can range from about 1 to about 50 grams per day, and preferably about 2 to about 16 grams per day in single or 2-4 divided doses.

In an alternative embodiment, the compositions used in the methods of the present invention can further comprise one or more ileal bile acid transport (“IBAT”) inhibitors (or apical sodium co-dependent bile acid transport (“ASBT”) inhibitors) coadministered with or in combination with the compound(s) of Formulae I-X discussed above. The IBAT inhibitors can inhibit bile acid transport to reduce LDL cholesterol levels. Non-limiting examples of suitable IBAT inhibitors include benzothiepines such as therapeutic compounds comprising a 2,3,4,5-tetrahydro-1-benzothiepine 1,1-dioxide structure such as are disclosed in PCT Patent Application WO 00/38727 which is incorporated herein by reference.

Generally, a total daily dosage of IBAT inhibitor(s) can range from about 0.01 to about 1000 mg/day, and preferably about 0.1 to about 50 mg/day in single or 2-4 divided doses.

In another alternative embodiment, the compositions used in the methods of the present invention can further comprise nicotinic acid (niacin) and/or derivatives thereof coadministered with or in combination with the compound(s) of Formulae I-X discussed above.

As used herein, “nicotinic acid derivative” means a compound comprising a pyridine-3-carboxylate structure or a pyrazine-2-carboxylate structure, including acid forms, salts, esters, zwitterions and tautomers, where available. Examples of nicotinic acid derivatives include niceritrol, nicofuranose and acipimox (5-methyl pyrazine-2-carboxylic acid 4-oxide). Nicotinic acid and its derivatives inhibit hepatic production of VLDL and its metabolite LDL and increases HDL and apo A-1 levels. An example of a suitable nicotinic acid product is NIASPAN® (niacin extended-release tablets) which are available from Kos.

Generally, a total daily dosage of nicotinic acid or a derivative thereof can range from about 500 to about 10,000 mg/day, preferably about 1000 to about 8000 mg/day, and more preferably about 3000 to about 6000 mg/day in single or divided doses.

In another alternative embodiment, the compositions used in the methods of the present invention can further comprise one or more AcylCoA:Cholesterol O-acyltransferase (“ACAT”) Inhibitors, which can reduce LDL and VLDL levels, coadministered with or in combination with the compound(s) of Formulae I-X discussed above. ACAT is an enzyme responsible for esterifying excess intracellular cholesterol and may reduce the synthesis of VLDL, which is a product of cholesterol esterification, and overproduction of apo B-100-containing lipoproteins.

Non-limiting examples of useful ACAT inhibitors include avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea). See P. Chang et al., “Current, New and Future Treatments in Dyslipidaemia and Atherosclerosis”, Drugs 2000 July;60(1); 55-93, which is incorporated by reference herein.

Generally, a total daily dosage of ACAT inhibitor(s) can range from about 0.1 to about 1000 mg/day in single or 2-4 divided doses.

In another alternative embodiment, the compositions used in the methods of the present invention can further comprise one or more Cholesteryl Ester Transfer Protein (“CETP”) Inhibitors coadministered with or in combination with the compound(s) of Formulae I-X discussed above. CETP is responsible for the exchange or transfer of cholesteryl ester carrying HDL and triglycerides in VLDL.

Non-limiting examples of suitable CETP inhibitors are disclosed in PCT Patent Application No. WO 00/38721 and U.S. Pat. No. 6,147,090, which are incorporated herein by reference. Pancreatic cholesteryl ester hydrolase (pCEH) inhibitors such as WAY-121898 also can be coadministered with or in combination with the fibric acid derivative(s) and sterol absorption inhibitor(s) discussed above.

Generally, a total daily dosage of CETP inhibitor(s) can range from about 0.01 to about 1000 mg/day, and preferably about 0.5 to about 20 mg/kg body weight/day in single or divided doses.

In another alternative embodiment, the compositions used in the methods of the present invention can further comprise probucol or derivatives thereof (such as AGI-1067 and other derivatives disclosed in U.S. Pat. Nos. 6,121,319 and 6,147,250), which can reduce LDL and HDL levels, coadministered with or in combination with the compound(s) of Formula I-X discussed above.

Generally, a total daily dosage of probucol or derivatives thereof can range from about 10 to about 2000 mg/day, and preferably about 500 to about 1500 mg/day in single or 2-4 divided doses.

In another alternative embodiment, the compositions used in the methods of the present invention can further comprise one or more low-density lipoprotein (LDL) receptor activators, coadministered with or in combination with the compound(s) of Formulae I-X discussed above. Non-limiting examples of suitable LDL-receptor activators include HOE-402, an imidazolidinyl-pyrimidine derivative that directly stimulates LDL receptor activity. See M. Huettinger et al., “Hypolipidemic activity of HOE-402 is Mediated by Stimulation of the LDL Receptor Pathway”, Arterioscler. Thromb. 1993; 13:1005-12.

Generally, a total daily dosage of LDL receptor activator(s) can range from about 1 to about 1000 mg/day in single or 2-4 divided doses.

In another alternative embodiment, the compositions used in the methods of the present invention can further comprise fish oil, which contains Omega 3 fatty acids (3-PUFA), which can reduce VLDL and triglyceride levels, coadministered with or in combination with the compound(s) of Formulae I-X discussed above. Generally, a total daily dosage of fish oil or Omega 3 fatty acids can range from about 1 to about 30 grams per day in single or 2-4 divided doses.

In another alternative embodiment, the compositions used in the methods of the present invention can further comprise natural water soluble fibers, such as psyllium, guar, oat and pectin, which can reduce cholesterol levels, coadministered with or in combination with the compound(s) of Formulae I-X discussed above. Generally, a total daily dosage of natural water soluble fibers can range from about 0.1 to about 10 grams per day in single or 2-4 divided doses.

In another alternative embodiment, the compositions used in the methods of the present invention can further comprise plant sterols, plant stanols and/or fatty acid esters of plant stanols, such as sitostanol ester used in BENECOL® margarine, which can reduce cholesterol levels, coadministered with or in combination with the compound(s) of Formulae I-X discussed above. Generally, a total daily dosage of plant sterols, plant stanols and/or fatty acid esters of plant stanols can range from about 0.5 to about 20 grams per day in single or 2-4 divided doses.

In another alternative embodiment, the compositions used in the methods of the present invention can further comprise one or more antioxidants, such as probucol, tocopherol, ascorbic acid, β-carotene and selenium, or vitamins such as vitamin B₆ or vitamin B₁₂, coadministered with or in combination with the compound(s) of Formulae I-X discussed above. Generally, a total daily dosage of antioxidants or vitamins can range from about 0.05 to about 10 grams per day in single or 2-4 divided doses.

In another alternative embodiment, the compositions used in the methods of the present invention can further comprise monocyte and macrophage inhibitors such as polyunsaturated fatty acids (PUFA), thyroid hormones including throxine analogues such as CGS-26214 (a thyroxine compound with a fluorinated ring), gene therapy and use of recombinant proteins such as recombinant apo E, coadministered with or in combination with the compound(s) of Formulae I-X discussed above. Generally, a total daily dosage of these agents can range from about 0.01 to about 1000 mg/day in single or 2-4 divided doses.

Also useful with the present invention are compositions or therapeutic combinations that further comprise hormone replacement agents and compositions. Useful hormone agents and compositions include androgens, estrogens, progestins, their pharmaceutically acceptable salts and derivatives. Combinations of these agents and compositions also are useful.

The dosage of androgen and estrogen combinations vary, desirably from about 1 mg to about 4 mg androgen and from about 1 mg to about 3 mg estrogen. Examples include, but are not limited to, androgen and estrogen combinations such as the combination of esterified estrogens (sodium estrone sulfate and sodium equilin sulfate) and methyltestosterone (17-hydroxy-17-methyl-, (17B)-androst-4-en-3-one) available from Solvay Pharmaceuticals, Inc., Marietta, Ga., under the tradename ESTRATEST.

Estrogens and estrogen combinations may vary in dosage from about 0.01 mg up to 8 mg, desirably from about 0.3 mg to about 3.0 mg. Examples of useful estrogens and estrogen combinations include:

(a) the blend of nine (9) synthetic estrogenic substances including sodium estrone sulfate, sodium equilin sulfate, sodium 17 α-dihydroequilin sulfate, sodium 17 α-estradiol sulfate, sodium 17 β-dihydroequilin sulfate, sodium 17 α-dihydroequilenin sulfate, sodium 17 β-dihydroequilenin sulfate, sodium equilenin sulfate and sodium 17 β-estradiol sulfate; available from Duramed Pharmaceuticals, Inc., Cincinnati, Ohio, under the tradename CENESTIN;

(b) ethinyl estradiol (19-nor-17 α-pregna-1,3,5(10)-trien-20-yne-3,17-diol; available by Schering Plough Corporation, Kenilworth, N.J., under the tradename ESTINYL;

(c) esterified estrogen combinations such as sodium estrone sulfate and sodium equilin sulfate; available from Solvay under the tradename ESTRATAB and from Monarch Pharmaceuticals, Bristol, Tenn., under the tradename MENEST;

(d) estropipate (piperazine estra-1,3,5(10)-trien-17-one, 3-(sulfooxy)-estrone sulfate); available from Pharmacia & Upjohn, Peapack, N.J., under the tradename OGEN and from Women First Health Care, Inc., San Diego, Calif., under the tradename ORTHO-EST; and

(e) conjugated estrogens (17 α-dihydroequilin, 17 α-estradiol, and 17 β-dihydroequilin); available from Wyeth-Ayerst Pharmaceuticals, Philadelphia, Pa., under the tradename PREMARIN.

Progestins and estrogens may also be administered with a variety of dosages, generally from about 0.05 to about 2.0 mg progestin and about 0.001 mg to about 2 mg estrogen, desirably from about 0.1 mg to about 1 mg progestin and about 0.01 mg to about 0.5 mg estrogen. Examples of progestin and estrogen combinations that may vary in dosage and regimen include:

(a) the combination of estradiol (estra-1, 3, 5 (10)-triene-3, 17 β-diol hemihydrate) and norethindrone (17 β-acetoxy-19-nor-17 α-pregn-4-en-20-yn-3-one); which is available from Pharmacia & Upjohn, Peapack, N.J., under the tradename ACTIVELLA;

(b) the combination of levonorgestrel (d(−)-13 β-ethyl-17 α-ethinyl-17 β-hydroxygon-4-en-3-one) and ethinyl estradial; available from Wyeth-Ayerst under the tradename ALESSE, from Watson Laboratories, Inc., Corona, Calif., under the tradenames LEVORA and TRIVORA, Monarch Pharmaceuticals, under the tradename NORDETTE, and from Wyeth-Ayerst under the tradename TRIPHASIL;

(c) the combination of ethynodiol diacetate (19-nor-17 α-pregn-4-en-20-yne-3 β, 17-diol diacetate) and ethinyl estradiol; available from G. D. Searle & Co., Chicago, Ill., under the tradename DEMULEN and from Watson under the tradename ZOVIA;

(d) the combination of desogestrel (13-ethyl-11-methylene-18,19-dinor-17 α-pregn-4-en-20-yn-17-ol) and ethinyl estradiol; available from Organon under the tradenames DESOGEN and MIRCETTE, and from Ortho-McNeil Pharmaceutical, Raritan, N.J., under the tradename ORTHO-CEPT;

(e) the combination of norethindrone and ethinyl estradiol; available from Parke-Davis, Morris Plains, N.J., under the tradenames ESTROSTEP and FEMHRT, from Watson under the tradenames MICROGESTIN, NECON, and TRI-NORINYL, from Ortho-McNeil under the tradenames MODICON and ORTHO-NOVUM, and from Warner Chilcott Laboratories, Rockaway, N.J., under the tradename OVCON;

(f) the combination of norgestrel ((±)-13-ethyl-17-hydroxy-18, 19-dinor-17 α-preg-4-en-20-yn-3-one) and ethinyl estradiol; available from Wyeth-Ayerst under the tradenames OVRAL and LO/OVRAL, and from Watson under the tradenames OGESTREL and LOW-OGESTREL;

(g) the combination of norethindrone, ethinyl estradiol, and mestranol (3-methoxy-19-nor-17 α-pregna-1,3,5(10)-trien-20-yn-17-ol); available from Watson under the tradenames BREVICON and NORINYL;

(h) the combination of 17 β-estradiol (estra-1,3,5(10)-triene-3,17 β-diol) and micronized norgestimate (17 α-17-(Acetyloxyl)-13-ethyl-18,19-dinorpregn-4-en-20-yn-3-one3-oxime); available from Ortho-McNeil under the tradename ORTHO-PREFEST;

(i) the combination of norgestimate (18,19-dinor-17-pregn-4-en-20-yn-3-one, 17-(acetyloxy)-13-ethyl-,oxime, (17(α)-(+)-) and ethinyl estradiol; available from Ortho-McNeil under the tradenames ORTHO CYCLEN and ORTHO TRI-CYCLEN; and

(j) the combination of conjugated estrogens (sodium estrone sulfate and sodium equilin sulfate) and medroxyprogesterone acetate (20-dione, 17-(acetyloxy)-6-methyl-, (6(α))-pregn-4-ene-3); available from Wyeth-Ayerst under the tradenames PREMPHASE and PREMPRO.

In general, a dosage of progestins may vary from about 0.05 mg to about 10 mg or up to about 200 mg if microsized progesterone is administered. Examples of progestins include norethindrone; available from ESI Lederle, Inc., Philadelphia, Pa., under the tradename AYGESTIN, from Ortho-McNeil under the tradename MICRONOR, and from Watson under the tradename NOR-QD; norgestrel; available from Wyeth-Ayerst under the tradename OVRETTE; micronized progesterone (pregn-4-ene-3, 20-dione); available from Solvay under the tradename PROMETRIUM; and medroxyprogesterone acetate; available from Pharmacia & Upjohn under the tradename PROVERA.

The compositions, therapeutic combinations or methods of the present invention can further comprise one or more obesity control medications. Useful obesity control medications include, but are not limited to, drugs that reduce energy intake or suppress appetite, drugs that increase energy expenditure and nutrient-partitioning agents. Suitable obesity control medications include, but are not limited to, noradrenergic agents (such as diethylpropion, mazindol, phenylpropanolamine, phentermine, phendimetrazine, phendamine tartrate, methamphetamine, phendimetrazine and tartrate); serotonergic agents (such as sibutramine, fenfluramine, dexfenfluramine, fluoxetine, fluvoxamine and paroxtine); thermogenic agents (such as ephedrine, caffeine, theophylline, and selective β3-adrenergic agonists); an alpha-blocking agent; a kainite or AMPA receptor antagonist; a leptin-lipolysis stimulated receptor; a phosphodiesterase enzyme inhibitor; a compound having nucleotide sequences of the mahogany gene; a fibroblast growth factor-10 polypeptide; a monoamine oxidase inhibitor (such as befloxatone, moclobemide, brofaromine, phenoxathine, esuprone, befol, toloxatone, pirlindol, amiflamine, sercloremine, bazinaprine, lazabemide, milacemide and caroxazone); a compound for increasing lipid metabolism (such as evodiamine compounds); and a lipase inhibitor (such as orlistat). Generally, a total dosage of the above-described obesity control medications can range from 1 to 3,000 mg/day, desirably from about 1 to 1,000 mg/day and more desirably from about 1 to 200 mg/day in single or 2-4 divided doses.

The compositions, therapeutic combinations or methods of the present invention can further comprise one or more blood modifiers. Useful blood modifiers include but are not limited to anti-coagulants (argatroban, bivalirudin, dalteparin sodium, desirudin, dicumarol, lyapolate sodium, nafamostat mesylate, phenprocoumon, tinzaparin sodium, warfarin sodium); antithrombotic (anagrelide hydrochloride, bivalirudin, cilostazol, dalteparin sodium, danaparoid sodium, dazoxiben hydrochloride, efegatran sulfate, enoxaparin sodium, fluretofen, ifetroban, ifetroban sodium, lamifiban, lotrafiban hydrochloride, napsagatran, orbofiban acetate, roxifiban acetate, sibrafiban, tinzaparin sodium, trifenagrel, abciximab, zolimomab aritox); fibrinogen receptor antagonists (roxifiban acetate, fradafiban, orbofiban, lotrafiban hydrochloride, tirofiban, xemilofiban, monoclonal antibody 7E3, sibrafiban); platelet inhibitors (cilostazol, clopidogrel bisulfate, epoprostenol, epoprostenol sodium, ticlopidine hydrochloride, aspirin, ibuprofen, naproxen, sulindae, idomethacin, mefenamate, droxicam, diclofenac, sulfinpyrazone, piroxicam, dipyridamole); platelet aggregation inhibitors (acadesine, beraprost, beraprost sodium, ciprostene calcium, itazigrel, lifarizine, lotrafiban hydrochloride, orbofiban acetate, oxagrelate, fradafiban, orbofiban, tirofiban, xemilofiban); hemorrheologic agents (pentoxifylline); lipoprotein associated coagulation inhibitor; Factor VIIa inhibitors (4H-31-benzoxazin-4-ones, 4H-3,1-benzoxazin-4-thiones, quinazolin-4-ones, quinazolin-4-thiones, benzothiazin-4-ones, imidazolyl-boronic acid-derived peptide analogues TFPI-derived peptides, naphthalene-2-sulfonic acid {1-[3-(aminoiminomethyl)-benzyl]-2-oxo-pyrrolidin-3-(S)-yl} amide trifluoroacetate, dibenzofuran-2-sulfonic acid {1-[3-(aminomethyl)-benzyl]-5-oxo-pyrrolidin-3-yl}-amide, tolulene-4-sulfonic acid {1-[3-(aminoiminomethyl)-benzyl]-2-oxo-pyrrolidin-3-(S)-yl}-amide trifluoroacetate, 3,4-dihydro-1H-isoquinoline-2-sulfonic acid {1-[3-(aminoiminomethyl)-benzyl]-2-oxo-pyrrolin-3-(S)-yl}-amide trifluoroacetate); Factor Xa inhibitors (disubstituted pyrazolines, disubstituted triazolines, substituted n-[(aminoiminomethyl)phenyl] propylamides, substituted n-[(aminomethyl)phenyl] propylamides, tissue factor pathway inhibitor (TFPI), low molecular weight heparins, heparinoids, benzimidazolines, benzoxazolinones, benzopiperazinones, indanones, dibasic (amidinoaryl) propanoic acid derivatives, amidinophenyl-pyrrolidines, amidinophenyl-pyrrolines, amidinophenyl-isoxazolidines, amidinoindoles, amidinoazoles, bis-arlysulfonylaminobenzamide derivatives, peptidic Factor Xa inhibitors).

The compositions, therapeutic combinations or methods of the present invention can further comprise one or more cardiovascular agents. Useful cardiovascular agents include but are not limited to calcium channel blockers (clentiazem maleate, amlodipine besylate, isradipine, nimodipine, felodipine, nilvadipine, nifedipine, teludipine hydrochloride, diltiazem hydrochloride, belfosdil, verapamil hydrochloride, fostedil); adrenergic blockers (fenspiride hydrochloride, labetalol hydrochloride, proroxan, alfuzosin hydrochloride, acebutolol, acebutolol hydrochloride, alprenolol hydrochloride, atenolol, bunolol hydrochloride, carteolol hydrochloride, celiprolol hydrochloride, cetamolol hydrochloride, cicloprolol hydrochloride, dexpropranolol hydrochloride, diacetolol hydrochloride, dilevalol hydrochloride, esmolol hydrochloride, exaprolol hydrochloride, flestolol sulfate, labetalol hydrochloride, levobetaxolol hydrochloride, levobunolol hydrochloride, metalol hydrochloride, metoprolol, metoprolol tartrate, nadolol, pamatolol sulfate, penbutolol sulfate, practolol, propranolol hydrochloride, sotalol hydrochloride, timolol, timolol maleate, tiprenolol hydrochloride, tolamolol, bisoprolol, bisoprolol fumarate, nebivolol); adrenergic stimulants; angiotensin converting enzyme (ACE) inhibitors (benazepril hydrochloride, benazeprilat, captopril, delapril hydrochloride, fosinopril sodium, libenzapril, moexipril hydrochloride, pentopril, perindopril, quinapril hydrochloride, quinaprilat, ramipril, spirapril hydrochloride, spiraprilat, teprotide, enalapril maleate, lisinopril, zofenopril calcium, perindopril erbumine); antihypertensive agents (althiazide, benzthiazide, captopril, carvedilol, chlorothiazide sodium, clonidine hydrochloride, cyclothiazide, delapril hydrochloride, dilevalol hydrochloride, doxazosin mesylate, fosinopril sodium, guanfacine hydrochloride, methyidopa, metoprolol succinate, moexipril hydrochloride, monatepil maleate, pelanserin hydrochloride, phenoxybenzamine hydrochloride, prazosin hydrochloride, primidolol, quinapril hydrochloride, quinaprilat, ramipril, terazosin hydrochloride, candesartan, candesartan cilexetil, telmisartan, amiodipine besylate, amlodipine maleate, bevantolol hydrochloride); angiotensin II receptor antagonists (candesartan, irbesartan, losartan potassium, candesartan cilexetil, telmisartan); anti-anginal agents (amlodipine besylate, amlodipine maleate, betaxolol hydrochloride, bevantolol hydrochloride, butoprozine hydrochloride, carvedilol, cinepazet maleate, metoprolol succinate, molsidomine, monatepil maleate, primidolol, ranolazine hydrochoride, tosifen, verapamil hydrochloride); coronary vasodilators (fostedil, azaclorzine hydrochloride, chromonar hydrochloride, clonitrate, diltiazem hydrochloride, dipyridamole, droprenilamine, erythrityl tetranitrate, isosorbide dinitrate, isosorbide mononitrate, lidoflazine, mioflazine hydrochloride, mixidine, molsidomine, nicorandil, nifedipine, nisoldipine, nitroglycerine, oxprenolol hydrochloride, pentrinitrol, perhexiline maleate, prenylamine, propatyl nitrate, terodiline hydrochloride, tolamolol, verapamil); diuretics (the combination product of hydrochlorothiazide and spironolactone and the combination product of hydrochlorothiazide and triamterene).

The compositions, therapeutic combinations or methods of the present invention can further comprise one or more antidiabetic medications for reducing blood glucose levels in a human. Useful antidiabetic medications include, but are not limited to, drugs that reduce energy intake or suppress appetite, drugs that increase energy expenditure and nutrient-partitioning agents. Suitable antidiabetic medications include, but are not limited to, sulfonylurea (such as acetohexamide, chlorpropamide, gliamilide, gliclazide, glimepiride, glipizide, glyburide, glibenclamide, tolazamide, and tolbutamide), meglitinide (such as repaglinide and nateglinide), biguanide (such as metformin and buformin), thiazolidinedione (such as troglitazone, rosiglitazone, pioglitazone, ciglitazone, englitazone, and darglitazone), alpha-glucosidase inhibitor (such as acarbose, miglitol, camiglibose, and voglibose), certain peptides (such as amlintide, pramlintide, exendin, and GLP-1 agonistic peptides), and orally administrable insulin or insulin composition for intestinal delivery thereof. Generally, a total dosage of the above-described antidiabetic medications can range from 0.1 to 1,000 mg/day in single or 2-4 divided doses.

Mixtures of any of the pharmacological or therapeutic agents described above can be included in the compositions used in the methods of the present invention.

The compositions used in the methods of the present invention can be administered to a subject in need of such treatment in a therapeutically effective amount to treat AD, regulate levels of amyloid β (Aβ) peptides and/or regulate the amount of ApoE isoform 4 in the bloodstream and/or brain. The compositions can be administered by any suitable means that produce contact of these compounds with the site of action in the body, for example in the plasma, liver, brain or small intestine of a subject.

The daily dosage for the various compositions and therapeutic combinations described above can be administered to a subject in a single dose or in multiple subdoses, as desired. Subdoses can be administered 2 to 6 times per day, for example. Sustained release dosages can be used. Where the compound(s) of Formulae I-X and cholesterol biosynthesis inhibitor(s) or lipid-lowering agent(s) are administered in separate dosages, the number of doses of each component given per day may not necessarily be the same, e.g., one component may have a greater duration of activity and will therefore need to be administered less frequently.

The compositions and therapeutic combinations of the present invention can further comprise one or more pharmaceutically acceptable carriers, one or more excipients and/or one or more additives. The pharmaceutical compositions can comprise about 1 to about 99 weight percent of active ingredient (one or more compounds of Formula I-X), and preferably about 5 to about 95 percent active ingredient.

Useful pharmaceutically acceptable carriers can be either solid, liquid or gas. Non-limiting examples of pharmaceutically acceptable carriers include solids and/or liquids such as magnesium carbonate, magnesium stearate, talc, sugar, lactose, ethanol, glycerol, water and the like. The amount of carrier in the treatment composition or therapeutic combination can range from about 5 to about 99 weight percent of the total weight of the treatment composition or therapeutic combination. Non-limiting examples of suitable pharmaceutically acceptable excipients and additives include non-toxic compatible fillers, binders such as starch, polyvinyl pyrrolidone or cellulose ethers, disintegrants such as sodium starch glycolate, crosslinked polyvinyl pyrrolidone or croscarmellose sodium, buffers, preservatives, anti-oxidants, lubricants, flavorings, thickeners, coloring agents, wefting agents such as sodium lauryl sulfate, emulsifiers and the like. The amount of excipient or additive can range from about 0.1 to about 95 weight percent of the total weight of the treatment composition or therapeutic combination. One skilled in the art would understand that the amount of carrier(s), excipients and additives (if present) can vary. Further examples of pharmaceutically acceptable carriers and methods of manufacture for various compositions can be found in A. Gennaro (ed.), Remington: The Science and Practice of Pharmacy, 20^{th} Edition, (2000), Lippincott Williams & Wilkins, Baltimore, Md.

Useful solid form preparations include powders, tablets, dispersible granules, capsules, cachets and suppositories. An example of a preparation of a preferred solid form dosage formulation is provided below.

Useful liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also useful are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably the compound is administered orally.

In another embodiment, the present invention provides the use of at least one compound represented by Formulae (I-X) for manufacture of a medicament (such as one of the compositions discussed above) for the treatment of Alzheimer's Disease, regulating production of or level of at least one amyloid β peptide and/or regulating the amount of ApoE isoform 4 in the bloodstream and/or brain of a subject.

The following formulation exemplifies one of the dosage forms of this invention. In the formulation, the term “Active Compound I” designates any of the compounds of Formulas I-X described herein above.

### EXAMPLE

Tablets

| No. | Ingredient | mg/tablet |
|---|---|---|
| 1 | Active Compound I | 10 |
| 2 | Lactose monohydrate NF | 55 |
| 3 | Microcrystalline cellulose NF | 20 |
| 4 | Povidone USP (K29-32) | 4 |
| 5 | Croscarmellose sodium NF | 8 |
| 6 | Sodium lauryl sulfate NF | 2 |
| 7 | Magnesium stearate NF | 1 |
| | Total | 100 |

Method of Manufacture

Mix Item No. 4 with purified water in suitable mixer to form binder solution. Spray the binder solution and then water over Items 1, 2, 6 and a portion of Item 5 in a fluidized bed processor to granulate the ingredients. Continue fluidization to dry the damp granules. Screen the dried granules and blend with Item No. 3 and the remainder of Item 5. Add Item No. 7 and mix. Compress the mixture to appropriate size and weight on a suitable tablet machine.

For coadministration in separate tablets or capsules, representative formulations comprising a compound of Formulae I-X such as are discussed above are well known in the art and representative formulations comprising a cholesterol absorption inhibitor and/or lipid lowering agent such as are discussed above are well known in the art. It is contemplated that where the two active ingredients are administered as a single composition, the dosage forms disclosed above for compounds of Formulae I-X may readily be modified using the knowledge of one skilled in the art.

Since one aspect of the present invention relates to treating AD, regulating production of or levels of amyloid β (Aβ) peptides and/or regulating the amount of ApoE isoform 4 in the bloodstream and/or brain by treatment with a combination of active ingredients wherein the active ingredients may be administered separately, the invention also relates to combining separate pharmaceutical compositions in kit form. That is, a kit is contemplated wherein two separate units are combined: a pharmaceutical composition comprising at least compound of Formulae I-X and a separate pharmaceutical composition comprising at least one cholesterol biosynthesis inhibitor or lipid-lowering agent as described above. The kit will preferably include directions for the administration of the separate components. The kit form is particularly advantageous when the separate components must be administered in different dosage forms (e.g., oral and parenteral) or are administered at different dosage intervals.

The methods of the present invention can be used for one or more of the following: to prevent, to treat AD, or ameliorate symptoms of AD, to regulate production of or levels of amyloid β (Aβ) peptides and/or regulate the amount of ApoE isoform 4 in the bloodstream and/or brain of a subject.

In one alternative embodiment, the human carries one or more mutations in the genes that encode β-amyloid precursor protein, presenilin-1 or presenilin-2.

In another alternative embodiment, the human carries the Apolipoprotein ε4 gene.

In another alternative embodiment, the human has a family history of Alzheimer's Disease or dementia illness.

In another alternative embodiment, the human has trisomy 21 (Down's Syndrome).

In another alternative embodiment, the subject has a normal or low serum total blood cholesterol level. In another embodiment, the serum total blood cholesterol level is less than about 200 mg/dl, more preferably less than about 180 and can range from about 150 to about 200 mg/dl. In another embodiment, the total LDL cholesterol level is less than about 100 mg/dl, more preferably less than about 90 mg/dl and can range from about 30 to about 100 mg/dl. Methods of measuring serum total blood cholesterol and total LDL cholesterol are well known to those skilled in the art and for example include those disclosed in PCT WO 99/38498 at page 11, incorporated by reference herein. Methods of determining levels of other sterols in serum are disclosed in H. Gylling et al., “Serum Sterols During Stanol Ester Feeding in a Mildly Hypercholesterolemic Population”, J. Lipid Res. 40: 593-600 (1999), incorporated by reference herein.

In another alternative embodiment, the subject has an elevated serum total blood cholesterol level. In another embodiment, the serum total cholesterol level is at least about 200 mg/dl, more preferably at least about 220 mg/dl and can range from about 200 to about 1000 mg/dl. In another alternative embodiment, the subject has an elevated total LDL cholesterol level. In another embodiment, the total LDL cholesterol level is greater than about 100 mg/dl, more preferably greater than about 110 mg/dl and can range from about 100 to about 1000 mg/dl.

In another alternative embodiment, the human is at least about 40 years of age. In another alternative embodiment, the human is at least about 60 years of age. In another embodiment, the human is at least about 70 years of age. Preferably the human is between about 60 and 100 years of age.

In another embodiment, the subject exhibits no symptoms of Alzheimer's Disease. In another embodiment, the subject is a human who is at least 40 years of age and exhibits no symptoms of Alzheimer's Disease. In another embodiment, the subject is a human who is at least 40 years of age and exhibits one or more symptoms of Alzheimer's Disease.

By using the methods of the present invention, the levels of amyloid β (Aβ) peptides in a subject's brain or blood can be reduced from levels prior to treatment from about 10 to about 100 percent, and preferably about 50 to about 100 percent.

In an alternative embodiment, the subject can have an elevated level of amyloid Aβ-42 peptide in the blood prior to treatment according to the present methods of greater than about 30 picomoles/liter (pM), preferably greater than about 35 pM, and more preferably greater than about 40 pM. In another embodiment, the elevated level of amyloid Aβ-42 peptide can range from about 30 pM to about 80 pM. One skilled in the art would understand that as the AD progresses, the measurable levels of amyloid β peptide may decrease slightly from elevated levels present before onset of the disease.

In an alternative embodiment, the subject can have an elevated level of amyloid Aβ-40 peptide in the blood prior to treatment according to the present methods of greater than about 200 picomoles/liter (pM), preferably greater than about 300 pM, and more preferably greater than about 400 pM. In another embodiment, the elevated level of amyloid Aβ-40 peptide can range from about 200 pM to about 800 pM.

In another embodiment, the subject can have an elevated level of amyloid Aβ-42 peptide in the brain prior to treatment according to the present methods of greater than about 50 picomoles per gram (pmol/g) of wet brain tissue weight, preferably greater than about 200 pmol/g, and more preferably greater than about 500 pmol/g. In another embodiment, the level of amyloid β peptide can range from about 50 pmol/g to about 10,000 pmol/g, and preferably about 500 pmol/g to about 10,000 pmol/g.

In another embodiment, the subject can have an elevated level of amyloid Aβ-40 peptide in the brain prior to treatment according to the present methods of greater than about 10 picomoles per gram (pmol/g) of wet brain tissue weight, preferably greater than about 50 pmol/g, and more preferably greater than about 100 pmol/g. In another embodiment, the level of amyloid β peptide can range from about 10 pmol/g to about 15,000 pmol/g.

The amount of amyloid β (Aβ) peptide in the brain or blood of a subject can be evaluated by enzyme-linked immunosorbent assay (ELISA) or quantitative immunolblotting test methods which are well known to those skilled in the art, such as is disclosed by Zhang et al., J. Biol. Chem. 274:8966-8972 (1999) and Zhang et al., Biochemistry 40:5049-5055 (2001). These tests are performed on samples of the brain or blood which have been prepared in a manner well known to one skilled in the art, for example as disclosed in the Example below. Another example of a useful method for measuring levels of amyloid β peptides is by Europium immunoassay (EIA), such as is disclosed in PCT WO 99/38498 at page 11, incorporated herein by reference.

In another embodiment, the amount of total ApoE in the bloodstream and/or brain of a subject can be reduced from levels prior to treatment by about 5 to about 75 percent, and preferably about 5 to about 50 percent. The amount of total ApoE can be measured in a manner well known to one skilled in the art, for example using an ELISA test kit such as Apo-Tek ApoE test kit that is available from Organon Teknica.

Illustrating the invention are the following examples which, however, are not to be considered as limiting the invention to their details. Unless otherwise indicated, all parts and percentages in the following examples, as well as throughout the specification, are by weight.

### EXAMPLES

Preparation of Compound of Formula (II)

Step 1): To a solution of (S)-4-phenyl-2-oxazolidinone (41 g, 0.25 mol) in CH₂Cl₂ (200 ml), was added 4-dimethylaminopyridine (2.5 g, 0.02 mol) and triethylamine (84.7 ml, 0.61 mol) and the reaction mixture was cooled to 0° C. Methyl-4-(chloroformyl)butyrate (50 g, 0.3 mol) was added as a solution in CH₂Cl₂ (375 ml) dropwise over 1 h, and the reaction was allowed to warm to 22° C. After 17 h, water and H₂SO₄ (2N, 100 ml), was added the layers were separated, and the organic layer was washed sequentially with NaOH (10%), NaCl (sat'd) and water. The organic layer was dried over MgSO₄ and concentrated to obtain a semicrystalline product.

Step 2): To a solution of TiCl₄ (18.2 ml, 0.165 mol) in CH₂Cl₂ (600 ml) at 0° C., was added titanium isopropoxide (16.5 ml, 0.055 mol). After 15 min, the product of Step 1 (49.0 g, 0.17 mol) was added as a solution in CH₂Cl₂ (100 ml). After 5 min., diisopropylethylamine (DIPEA) (65.2 ml, 0.37 mol) was added and the reaction mixture was stirred at 0° C. for 1 h, the reaction mixture was cooled to −20° C., and 4-benzyloxybenzylidine(4-fluoro)aniline (114.3 g, 0.37 mol) was added as a solid. The reaction mixture was stirred vigorously for 4 h at −20° C., then acetic acid was added as a solution in CH₂Cl₂ dropwise over 15 min, the reaction mixture was allowed to warm to 0° C., and H₂SO₄ (2N) was added. The reaction mixture was stirred an additional 1 h, the layers were separated, washed with water, separated and the organic layer was dried. The crude product was crystallized from ethanol/water to obtain the pure intermediate.

Step 3): To a solution of the product of Step 2 (8.9 g, 14.9 mmol) in toluene (100 ml) at 50° C., was added N,O-bis(trimethylsilyl)acetamide (BSA) (7.50 ml, 30.3 mmol). After 0.5 h, solid TBAF (0.39 g, 1.5 mmol) was added and the reaction mixture stirred at 50° C. for an additional 3 h. The reaction mixture was cooled to 22° C., CH₃OH (10 ml), was added. The reaction mixture was washed with HCl (1N), NaHCO₃ (1N) and NaCl (sat'd.), and the organic layer was dried over MgSO₄.

Step 4): To a solution of the product of Step 3 (0.94 g, 2.2 mmol) in CH₃OH (3 ml), was added water (1 ml) and LiOH.H₂O (102 mg, 2.4 mmole). The reaction mixture was stirred at 22° C. for 1 h and then additional LiOH.H₂O (54 mg, 1.3 mmole) was added. After a total of 2 h, HCl (1N) and EtOAc was added, the layers were separated, the organic layer was dried and concentrated in vacuo. To a solution of the resultant product (0.91 g, 2.2 mmol) in CH₂Cl₂ at 22° C., was added ClCOCOCl (0.29 ml, 3.3 mmol) and the mixture stirred for 16 h. The solvent was removed in vacuo.

Step 5): To an efficiently stirred suspension of 4-fluorophenylzinc chloride (4.4 mmol) prepared from 4-fluorophenylmagnesium bromide (1M in THF, 4.4 ml, 4.4 mmol) and ZnCl₂ (0.6 g, 4.4 mmol) at 4° C., was added tetrakis(triphenyl-phosphine)palladium (0.25 g, 0.21 mmol) followed by the product of Step 4 (0.94 g, 2.2 mmol) as a solution in THF (2 ml). The reaction was stirred for 1 h at 0° C. and then for 0.5 h at 22° C. HCl (1N, 5 ml) was added and the mixture was extracted with EtOAc. The organic layer was concentrated to an oil and purified by silica gel chromatography to obtain 1-(4-fluorophenyl)-4(S)-(4-hydroxyphenyl)-3(R)-(3-oxo-3-phenylpropyl)-2-azetidinone:

HRMS calc'd for C₂₄H₁₉F₂NO₃=408.1429, found 408.1411.

Step 6): To the product of Step 5 (0.95 g, 1.91 mmol) in THF (3 ml), was added (R)-tetrahydro-1-methyl-3,3-diphenyl-1H,3H-pyrrolo-[1,2-c][1,3,2] oxazaborole (120 mg, 0.43 mmol) and the mixture was cooled to −20° C. After 5 min, borohydride-dimethylsulfide complex (2M in THF, 0.85 ml, 1.7 mmol) was added dropwise over 0.5 h. After a total of 1.5 h , CH₃OH was added followed by HCl (1 N) and the reaction mixture was extracted with EtOAc to obtain 1-(4-fluorophenyl)-3(R)-[3(S)-(4-fluorophenyl)-3-hydroxypropyl)]-4(S)-[4-(phenylmethoxy)phenyl]-2-azetidinone (compound 6A-1) as an oil. ¹H in CDCl₃ d H3=4.68. J=2.3 Hz. Cl (M⁺H) 500.

Use of (S)-tetra-hydro-1-methyl-3,3-diphenyl-1H,3H-pyrrolo-[1,2-c][1,3,2] oxazaborole gives the corresponding 3(R)-hydroxypropyl azetidinone (compound 6B-1). ¹H in CDCl₃ d H3=4.69. J=2.3 Hz. Cl (M⁺H) 500.

To a solution of compound 6A-1 (0.4 g, 0.8 mmol) in ethanol (2 ml), was added 10% Pd/C (0.03 g) and the reaction mixture was stirred under a pressure (60 psi) of H₂ gas for 16 h. The reaction mixture was filtered and the solvent was concentrated to obtain compound 6A. Mp 164-166° C.; Cl (M⁺H) 410. [α]_{D}²⁵=−28.1° (c 3, CH₃OH). Elemental analysis calc'd for C₂₄H₂₁F₂NO₃: C 70.41; H 5.17; N 3.42; found C 70.25; H 5.19; N 3.54.

Similarly treat compound 6B-1 to obtain compound 6B.

Mp 129.5-132.5° C.; Cl (M⁺H) 410. Elemental analysis calc'd for C₂₄H₂₁F₂NO₃: C 70.41; H 5.17; N 3.42; found C 70.30; H 5.14; N 3.52.

Step 6′ (Alternative): To a solution of the product of Step 5 (0.14 g, 0.3 mmol) in ethanol (2 ml), was added 10% Pd/C (0.03 g) and the reaction was stirred under a pressure (60 psi) of H₂ gas for 16 h. The reaction mixture was filtered and the solvent was concentrated to afford a 1:1 mixture of compounds 6A and 6B.

Hypothetical in vivo Evaluation

The compound of Formula VIa is administered to transgenic mice (about 5 weeks of age) overexpressing the β-amyloid precursor protein. The mice are CRND8 mice available from University of Toronto, described in Janus et al., Nature (December 2000) and Chishti et al., JBC Online 2001. The compound is administered at a dosage of 30 mg/kg/day in the diet over a period of 5-11 weeks. After the specified period of compound administration, mice are sacrificed by CO₂ asphyxiation and the brains are removed and placed in phosphate buffered saline. One cortex is dissected away from the remainder of one hemisphere and is homogenized in 300 μl of sucrose homogenization buffer (20 mM Tris base, 250 mM sucrose, 1 mM EDTA (ethylene diamine tetraacetic acid) and 1 mM EGTA). The residual brain hemisphere (minus cortex) is used to measure CNS cholesterol and triglycerides using methods discussed above. The remaining intact hemisphere is snap-frozen on dry ice and cryostat sectioned. Sections are stained for Aβ-containing plaques using monoclonal anti-human Aβ antibody 6F/3D (Dako) and Cy-3 conjugated secondary antibody (available from Jackson Immunological Research). Plaque number and size are quantitated from microscopic images of stained sections visualized with fluorescent light using Image-Pro Plus (available from Media Cybernetics).

Soluble Aβ peptide is extracted by mixing 50 μl of the cortex homogenate with 50 μl of sucrose homogenization buffer and centrifuging the mixture at 100,000 g for 1 hour at 4° C. The pellet is then resuspended in 100 μl of cell lysis buffer (1% sodium dodecyl sulfate, 10 mM Tris pH 7.4, 150 mM NaCl, 5 mM EDTA) and the resulting mixture is centrifuged at 100,000 g for 1 hour at 4° C. The supernatant is then removed, tested for protein concentration and used for measurement of Aβ peptide as described below.

Aβ peptide deposited in plaques is extracted by mixing 100 μl of the cortex homogenate with 233 ml of cold (4° C.) formic acid (70% final concentration of formic acid) and sonicating the mixture for 1 minute on ice using a Fisher Sonic Dismembrator Model F60 at Setting 5. The mixture is then centrifuged at 100,000 g for 1 hour at 4° C. and 210 μl of the supernatant is then diluted with 210 μl of formic acid neutralization solution (1M Tris base, 0.5 M Na₂HPO₄). This solution is used for measurement of Aβ peptide as described below.

Plasma Aβ peptide is measured directly without further treatment of plasma samples. Cholesterol and triglyceride levels are measured from aliquots of these plasma samples.

Aβ40 and Aβ42 peptides are measured independently by enzyme-linked immunosorbent assay. Antibodies 4G8 and G2-10 are used for the measurement of Aβ40 and antibodies 4G8 and G2-11 are used for the measurement of Aβ42 (Ida et al., J. Biol. Chem. 271:22908-22914, 1996). Detection of ELISA assays is accomplished by electrochemiluminescent detection using Igen M8 which is available from Igen International, Gaithersburg, Md.).

It will be appreciated by those skilled in the art that changes could be made to the embodiments described above without departing from the broad inventive concept thereof. It is understood, therefore, that this invention is not limited to the particular embodiments disclosed, but it is intended to cover modifications which are within the spirit and scope of the invention, as defined by the appended claims.

Therefore, we claim:

1. A method of preventing, treating, or ameliorating symptoms of Alzheimer's Disease comprising the step of administering to a subject in need of such treatment an effective amount of a composition comprising at least one compound represented by Formula (I): 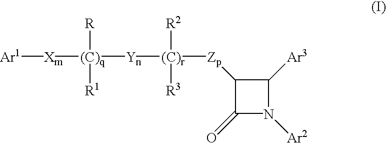
or a pharmaceutically acceptable salt thereof or solvate thereof, wherein:
Ar¹ and Ar² are independently selected from the group consisting of aryl and R⁴-substituted aryl;
Ar³ is aryl or R⁵-substituted aryl;
X, Y and Z are independently selected from the group consisting of —CH₂—, —CH(lower alkyl)- and —C(dilower alkyl)-;
R and R² are independently selected from the group consisting of —OR⁶, —O(CO)R⁶, —O(CO)OR⁹ and —O(CO)NR⁶R⁷;
R¹ and R³ are independently selected from the group consisting of hydrogen, lower alkyl and aryl;
q is 0 or 1;
r is 0 or 1;
m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl to prevent, treat, or ameliorate symptoms of Alzheimer's Disease in the subject.

2. The method according to claim 1, wherein the subject exhibits no symptoms of Alzheimer's Disease.

3. The method according to claim 1, wherein the subject has Alzheimer's Disease.

4. The method according to claim 1, wherein the subject has a family history of Alzheimer's Disease or dementia illness.

5. The method according to claim 1, wherein the subject is a human and has trisomy 21 (Down's Syndrome).

6. The method according to claim 1, wherein the subject is a human.

7. The method according to claim 6, wherein the human carries one or more mutations in the genes that encode β-amyloid precursor protein, presenilin-1 or presenilin-2.

8. The method according to claim 6, wherein the human carries the Apolipoprotein E4 gene.

9. The method according to claim 1, wherein the subject has an elevated level of amyloid β peptide in the bloodstream and/or the brain.

10. The method according to claim 9, wherein the subject has an elevated level of amyloid β 42 peptide in the bloodstream and/or the brain.

11. The method according to claim 9, wherein the level of amyloid β (Aβ) peptides in the bloodstream is reduced from about 10 to about 100 percent from a level of amyloid β (Aβ) peptides prior to administration of the composition.

12. The method according to claim 9, wherein the subject has an level of amyloid Aβ-42 peptide greater than about 30 pM in the bloodstream.

13. The method according to claim 12, wherein the subject has an level of amyloid Aβ-42 peptide greater than about 40 pM in the bloodstream.

14. The method according to claim 12, wherein the subject has an level of amyloid Aβ-42 peptide ranging from about 30 pM to about 80 pM in the bloodstream.

15. The method according to claim 9, wherein the subject has an level of amyloid Aβ-40 peptide greater than about 200 pM in the bloodstream.

16. The method according to claim 15, wherein the subject has an level of amyloid Aβ-40 peptide greater than about 400 pM in the bloodstream.

17. The method according to claim 15, wherein the subject has an level of amyloid Aβ-40 peptide ranging from about 200 pM to about 800 pM in the bloodstream.

18. The method according to claim 9, wherein the subject has an level of amyloid Aβ-42 peptide of greater than about 50 pmol/gram of wet brain tissue.

19. The method according to claim 9, wherein the subject has an level of amyloid Aβ-40 peptide of greater than about 10 pmol/gram of wet brain tissue.

20. The method according to claim 1, wherein the subject has an elevated blood cholesterol level.

21. The method according to claim 1, wherein the total serum cholesterol level of the subject is at least about 200 mg/dl.

22. The method according to claim 1, wherein the total LDL cholesterol level of the subject is greater than about 100 mg/dl.

23. The method according to claim 6, wherein the human is greater than about 40 years of age.

24. The method according to claim 7, wherein the human is greater than about 60 years of age.

25. The method according to claim 1, wherein the compound is represented by Formula (II) below:

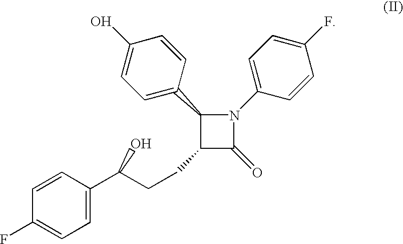

26. The method according to claim 1, wherein the compound is represented by Formula (VIa) below:

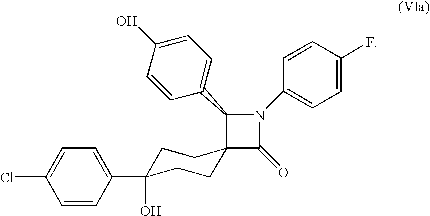

27. The method according to claim 1, wherein the compound is administered to the subject in an amount ranging from about 0.1 to about 1000 milligrams of compound per day.

28. The method according to claim 1, wherein the composition further comprises at least one cholesterol biosynthesis inhibitor.

29. The method according to claim 28, wherein the at least one cholesterol biosynthesis inhibitor comprises at least one HMG CoA reductase inhibitor.

30. The method according to claim 29, wherein the at least one HMG CoA reductase inhibitor is selected from lovastatin, pravastatin, fluvastatin, simvastatin, atorvastatin, cerivastatin, rosuvastatin or mixtures thereof.

31. The method according to claim 30, wherein the at least one HMG CoA reductase inhibitor is simvastatin.

32. The method according to claim 1, wherein the composition further comprises at least one fibric acid derivative.

33. The method according to claim 1, wherein the composition further comprises at least one bile acid sequestrant.

34. The method according to claim 1, wherein the composition further comprises nicotinic acid or a derivative thereof.

35. The method according to claim 1, wherein the composition further comprises at least one AcylCoA:Cholesterol O-acyltransferase Inhibitor.

36. The method according to claim 1, wherein the composition further comprises probucol or a derivative thereof.

37. The method according to claim 1, wherein the composition further comprises at least one low-density lipoprotein receptor activator.

38. The method according to claim 1, wherein the composition further comprises at least one Omega 3 fatty acid.

39. The method according to claim 1, wherein the composition further comprises at least one natural water soluble fiber.

40. The method according to claim 1, wherein the composition further comprises at least one of plant sterols, plant stanols or fatty acid esters of plant stanols.

41. The method according to claim 1, wherein the composition further comprises at least one antioxidant or vitamin.

42. The method according to claim 1, wherein the composition further comprises at least one Alzheimer's treatment different from compound I above.

43. The method according to claim 1, wherein the composition further comprises at least one Alzheimer's treatment different from compound I above selected from the group consisting of cholinesterase inhibitors, muscarinic receptor antagonists, M2 muscarinic receptor antagonists, acetylcholine release stimulators, choline uptake stimulators, nicotinic cholinergic receptor antagonists, anti-Aβ vaccines, γ-secretase inhibitors, β-inhibitors, amyloid aggregation inhibitors, amyloid precursor protein antisense oligonucleotides, monoamine reuptake inhibitors, human stem cells, gene therapy, nootropic agents, AMPA receptor ligands, growth factors or growth factor receptor agonists, anti-inflammatory agents, free radical scavengers/antioxidants, superoxide dismutase stimulators, calcium channel blockers, apoptosis inhibitors, caspase inhibitors, monoamine oxidase inhibitors, estrogens, NMDA receptor antagonists, Jun N-terminal kinase (JNK) inhibitors, copper/zinc chelators, 5-HT1 a receptor agonists, NGF stimulators, neuroprotective agents, H3 histamine receptor antagonists, calpain inhibitors, poly ADP ribose polymerase inhibitors, prolylendopeptidase inhibitors, calcium modulators, corticortropin releasing factor antagonists, corticortropin releasing factor binding protein inhibitor, GABA modulators, GABA-A receptor antagonists, GABA-B receptor antagonists, neuroimmunophilin ligands, sigma receptor ligands, galanin receptor ligands, imidazoline/alpha adrenergic receptor antagonists, vasoactive intestinal peptide receptor agonists, benzodiazepine inverse agonists, cannabinoid receptor agonists, thyrotropin releasing hormone receptor agonists, protein kinase C inhibitors, 5-HT3 antagonists, prostaglandin receptor antagonists, topoisomerase II inhibitors, steroid receptor agonists, corticosteroid receptor antagonists, nitric oxide modulators, RAGE inhibitors, dopamine receptor agonists, and combinations thereof.

44. A method of preventing, treating, or ameliorating symptoms of Alzheimer's Disease comprising the step of administering to a subject in need of such treatment an effective amount of a composition comprising a compound represented by Formula (II) below: 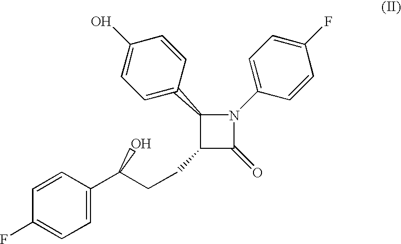
to prevent, treat, or ameliorate symptoms of Alzheimer's Disease in the subject.

45. A method of regulating the production of at least one amyloid β peptide in a subject or regulating a level of at least one amyloid β peptide in bloodstream and/or brain of a subject, comprising the step of administering to a subject in need of such treatment an effective amount of a composition comprising at least one compound represented by Formula (I): 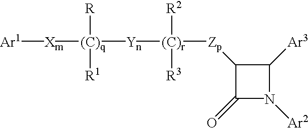
or a pharmaceutically acceptable salt thereof or solvate thereof, wherein:
Ar¹ and Ar² are independently selected from the group consisting of aryl and R⁴-substituted aryl;
Ar³ is aryl or R⁵-substituted aryl;
X, Y and Z are independently selected from the group consisting of —CH₂—, —CH(lower alkyl)- and —C(dilower alkyl)-;
R and R² are independently selected from the group consisting of —OR⁶, —O(CO)R⁶, —O(CO)OR⁹ and —O(CO)NR⁶R⁷;
R¹ and R³ are independently selected from the group consisting of hydrogen, lower alkyl and aryl;
q is 0 or 1;
r is 0 or 1;
m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl, to regulate the production or level of at least one amyloid β peptide in the subject.

46. A method of regulating the amount of ApoE isoform 4 in the bloodstream and/or brain of the subject comprising the step of administering to a subject in need of such treatment an effective amount of a composition comprising at least one compound represented by Formula (I): 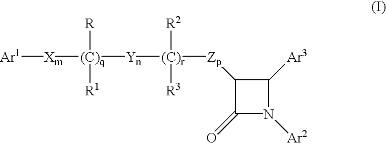
or a pharmaceutically acceptable salt thereof or solvate thereof, wherein:
Ar¹ and Ar² are independently selected from the group consisting of aryl and R⁴-substituted aryl;
Ar³ is aryl or R⁵-substituted aryl;
X, Y and Z are independently selected from the group consisting of —CH₂—, —CH(lower alkyl)- and —C(dilower alkyl)-;
R and R² are independently selected from the group consisting of —OR⁶, —O(CO)R⁶, —O(CO)OR⁹ and —O(CO)NR⁶R⁷;
R¹ and R³ are independently selected from the group consisting of hydrogen, lower alkyl and aryl;
q is 0 or 1;
r is 0 or 1;
m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl, to regulate the amount of ApoE isoform 4 in the bloodstream and/or brain of the subject.

47. A method of preventing, treating, or ameliorating symptoms of Alzheimer's Disease comprising the step of administering to a subject in need of such treatment an effective amount of a composition comprising at least one compound represented by Formula (III): 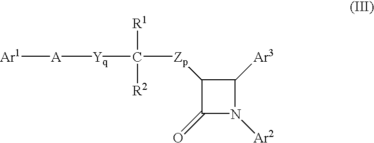
or a pharmaceutically acceptable salt thereof or solvate thereof, wherein, in Formula (III) above:
Ar¹ is R³-substituted aryl;
Ar² is R⁴-substituted aryl;
Ar³ is R⁵-substituted aryl;
Y and Z are independently selected from the group consisting of —CH₂—, —CH(lower alkyl)- and —C(dilower alkyl)-;
A is selected from —O—, —S—, —S(O)— or —S(O)₂—;
R¹ is selected from the group consisting of —OR⁶, —O(CO)R⁶, —O(CO)OR⁹ and —O(CO)NR⁶R⁷; R² is selected from the group consisting of hydrogen, lower alkyl and aryl; or R¹ and R² together are ═O;
q is 1, 2 or 3;
p is 0, 1, 2, 3 or 4;
R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;
R³ and R⁴ are independently 1-3 substituents independently selected from the group consisting of R⁵, hydrogen, p-lower alkyl, aryl, —NO₂, —CF₃ and p-halogeno;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl to prevent, treat, or ameliorate symptoms of Alzheimer's Disease in the subject.

48. A method of regulating the production of at least one amyloid β peptide in a subject or regulating a level of at least one amyloid β peptide in bloodstream and/or brain of a subject, comprising the step of administering to a subject in need of such treatment an effective amount of a composition comprising at least one compound represented by Formula (III): 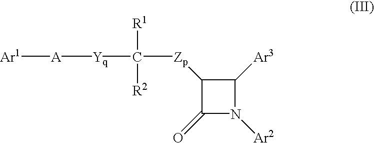
or a pharmaceutically acceptable salt thereof or solvate thereof, wherein, in Formula (III) above:
Ar¹ is R³-substituted aryl;
Ar² is R⁴-substituted aryl;
Ar³ is R⁵-substituted aryl;
Y and Z are independently selected from the group consisting of —CH₂—, —CH(lower alkyl)- and —C(dilower alkyl)-;
A is selected from —O—, —S—, —S(O)— or —S(O)₂—;
R¹ is selected from the group consisting of —OR⁶, —O(CO)R⁶, —O(CO)OR⁹ and —O(CO)NR⁶R⁷; R² is selected from the group consisting of hydrogen, lower alkyl and aryl; or R¹ and R² together are ═O;
q is 1, 2 or 3;
p is 0, 1, 2, 3 or 4;
R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;
R³ and R⁴ are independently 1-3 substituents independently selected from the group consisting of R⁵, hydrogen, p-lower alkyl, aryl, —NO₂, —CF₃ and p-halogeno;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl, to regulate the production or level of at least one amyloid β peptide in the subject.

49. A method of regulating the amount of ApoE isoform 4 in the bloodstream and/or brain of the subject comprising the step of administering to a subject in need of such treatment an effective amount of a composition comprising at least one compound represented by Formula (III): 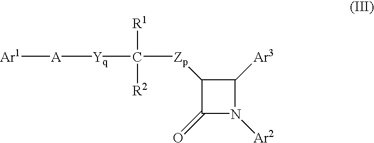
or a pharmaceutically acceptable salt thereof or solvate thereof, wherein, in Formula (III) above:
Ar¹ is R³-substituted aryl;
Ar² is R⁴-substituted aryl;
Ar³ is R⁵-substituted aryl;
Y and Z are independently selected from the group consisting of —CH₂—, —CH(lower alkyl)- and —C(dilower alkyl)-;
A is selected from —O—, —S—, —S(O)— or —S(O)₂—;
R¹ is selected from the group consisting of —OR⁶, —O(CO)R⁶, —O(CO)OR⁹ and —O(CO)NR⁶R⁷; R² is selected from the group consisting of hydrogen, lower alkyl and aryl; or R¹ and R² together are ═O;
q is 1, 2 or 3;
p is 0, 1, 2, 3 or 4;
R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;
R³ and R⁴ are independently 1-3 substituents independently selected from the group consisting of R⁵, hydrogen, p-lower alkyl, aryl, —NO₂, —CF₃ and p-halogeno;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl, to regulate the amount of ApoE isoform 4 in the bloodstream and/or brain of the subject.

50. A method of preventing, treating, or ameliorating symptoms of Alzheimer's Disease comprising the step of administering to a subject in need of such treatment an effective amount of a composition comprising at least one compound represented by Formula (IV): 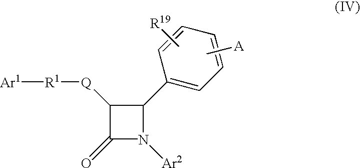
or a pharmaceutically acceptable salt thereof or solvate thereof, wherein, in Formula (IV) above:
A is selected from the group consisting of R²-substituted heterocycloalkyl, R²-substituted heteroaryl, R²-substituted benzofused heterocycloalkyl, and R²-substituted benzofused heteroaryl;
Ar¹ is aryl or R³-substituted aryl;
Ar² is aryl or R⁴-substituted aryl;
Q is a bond or, with the 3-position ring carbon of the azetidinone, forms the spiro group 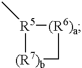
and
R¹ is selected from the group consisting of:
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;
R⁵ is selected from: 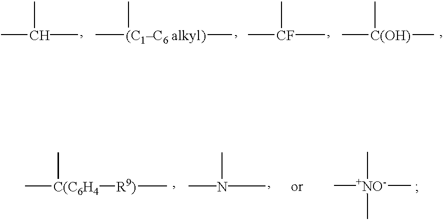
R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)- group;
a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷'s can be the same or different;
and when Q is a bond, R¹ also can be selected from: 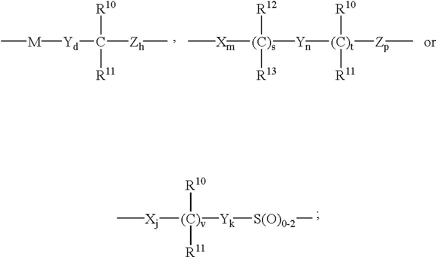
where M is —O—, —S—, —S(O)— or —S(O)₂—;
X, Y and Z are independently selected from the group consisting of —CH₂—, —CH(C₁-C₆ alkyl)- and —C(di-(C₁-C₆) alkyl);
R¹⁰ and R¹² are independently selected from the group consisting of —OR¹⁴, —O(CO)R¹⁴, —O(CO)OR¹⁶ and —O(CO)NR¹⁴R¹⁵;
R¹¹ and R¹³ are independently selected from the group consisting of hydrogen, (C₁-C₆)alkyl and aryl; or R¹⁰ and R¹¹ together are ═O, or R¹² and R¹³ together are ═O;
d is 1, 2 or 3;
h is 0, 1, 2, 3 or 4;
s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R¹⁶, —SO₂NR¹⁴R¹⁵ and —(C₁-C₆ alkylene)COOR¹⁴; when R² is a substituent on a heterocycloalkyl ring, R² is as defined, or is ═O or 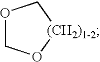
and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸, 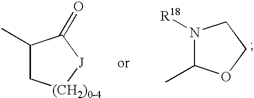
wherein J is —O—, —NH—, —NR¹⁸— or —CH₂—;
R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR¹⁶, —NR¹⁴(CO)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;
R⁸ is hydrogen, (C₁-C₆)alkyl, aryl (C₁-C₆)alkyl, —C(O)R¹⁴ or —COOR¹⁴;
R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;
R¹⁴ and R¹⁵ are independently selected from the group consisting of hydrogen, (C₁-C₆)alkyl, aryl and aryl-substituted (C₁-C₆)alkyl;
R¹⁶ is (C₁-C₆)alkyl, aryl or R¹⁷-substituted aryl;
R¹⁸ is hydrogen or (C₁-C₆)alkyl; and
R¹⁹ is hydrogen, hydroxy or (C₁-C₆)alkoxy, to prevent, treat, or ameliorate symptoms of Alzheimer's Disease in the subject.

51. A method of regulating the production of at least one amyloid β peptide in a subject or regulating a level of at least one amyloid β peptide in bloodstream and/or brain of a subject, comprising the step of administering to a subject in need of such treatment an effective amount of a composition comprising at least one compound represented by Formula (IV): 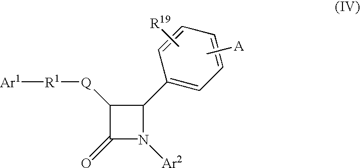
or a pharmaceutically acceptable salt thereof or solvate thereof, wherein, in Formula (IV) above:
A is selected from the group consisting of R²-substituted heterocycloalkyl, R²-substituted heteroaryl, R²-substituted benzofused heterocycloalkyl, and R²-substituted benzofused heteroaryl;
Ar¹ is aryl or R³-substituted aryl;
Ar² is aryl or R⁴-substituted aryl;
Q is a bond or, with the 3-position ring carbon of the azetidinone, forms the spiro group 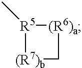
and
R¹ is selected from the group consisting of:
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;
R⁵ is selected from: 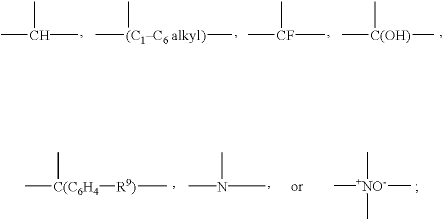
R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)- group;
a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷'s can be the same or different;
and when Q is a bond, R¹ also can be selected from: 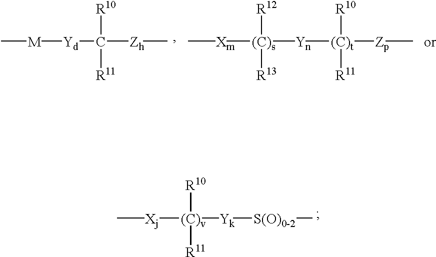
where M is —O—, —S—, —S(O)— or —S(O)₂—;
X, Y and Z are independently selected from the group consisting of —CH₂—, —CH(C₁-C₆ alkyl)- and —C(di-(C₁-C₆) alkyl);
R¹⁰ and R¹² are independently selected from the group consisting of —OR¹⁴, —O(CO)R¹⁴, —O(CO)OR¹⁶ and —O(CO)NR¹⁴R¹⁵;
R¹¹ and R¹³ are independently selected from the group consisting of hydrogen, (C₁-C₆)alkyl and aryl; or R¹⁰ and R¹¹ together are ═O, or R¹² and R¹³ together are ═O;
d is 1, 2 or 3;
h is 0, 1, 2, 3 or 4;
s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R¹⁶, —SO₂NR¹⁴R¹⁵ and —(C₁-C₆ alkylene)COOR¹⁴; when R² is a substituent on a heterocycloalkyl ring, R² is as defined, or is ═O or 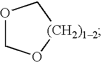
and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸, 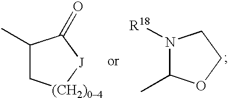
wherein J is —O—, —NH—, —NR¹⁸— or —CH₂—;
R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR¹⁶, —NR¹⁴(CO)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;
R⁸ is hydrogen, (C₁-C₆)alkyl, aryl (C₁-C₆)alkyl, —C(O)R¹⁴ or —COOR¹⁴;
R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;
R¹⁴ and R¹⁵ are independently selected from the group consisting of hydrogen, (C₁-C₆)alkyl, aryl and aryl-substituted (C₁-C₆)alkyl;
R¹⁶ is (C₁-C₆)alkyl, aryl or R¹⁷-substituted aryl;
R¹⁸ is hydrogen or (C₁-C₆)alkyl; and
R¹⁹ is hydrogen, hydroxy or (C₁-C₆)alkoxy, to regulate the production or level of at least one amyloid β peptide in the subject.

52. A method of regulating the amount of ApoE isoform 4 in the bloodstream and/or brain of the subject comprising the step of administering to a subject in need of such treatment an effective amount of a composition comprising at least one compound represented by Formula (IV): 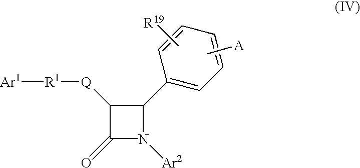
or a pharmaceutically acceptable salt thereof or solvate thereof, wherein, in Formula (IV) above:
A is selected from the group consisting of R²-substituted heterocycloalkyl, R²-substituted heteroaryl, R²-substituted benzofused heterocycloalkyl, and R²-substituted benzofused heteroaryl;
Ar¹ is aryl or R³-substituted aryl;
Ar² is aryl or R⁴-substituted aryl;
Q is a bond or, with the 3-position ring carbon of the azetidinone, forms the spiro group 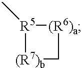
and
R¹ is selected from the group consisting of:
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;
R⁵ is selected from: 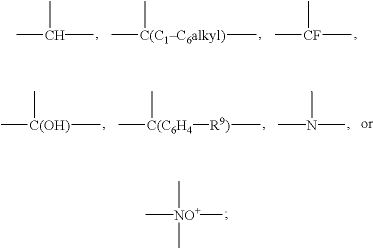
R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)- group;
a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷'s can be the same or different;
and when Q is a bond, R¹ also can be selected from: 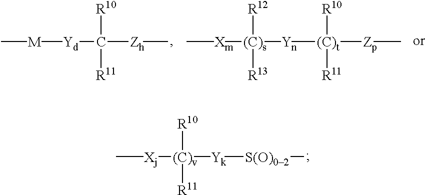
where M is —O—, —S—, —S(O)— or —S(O)₂—;
X, Y and Z are independently selected from the group consisting of —CH₂—, —CH(C₁-C₆ alkyl)- and —C(di-(C₁-C₆) alkyl);
R¹⁰ and R¹² are independently selected from the group consisting of —OR¹⁴, —O(CO)OR¹⁶ and —O(CO)NR¹⁴R¹⁵;
R¹¹ and R¹³ are independently selected from the group consisting of hydrogen, (C₁-C₆)alkyl and aryl; or R¹⁰ and R¹¹ together are ═O, or R¹² and R¹³ together are ═O;
d is 1, 2 or 3;
h is 0, 1, 2, 3 or 4;
s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-,—NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R¹⁶, —SO₂NR¹⁴R¹⁵ and —(C₁-C₆ alkylene)COOR¹⁴; when R² is a substituent on a heterocycloalkyl ring, R² is as defined, or is ═O or 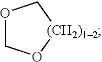
and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸, 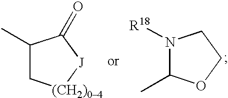
wherein J is —O—, —NH—, —NR¹⁸— or —CH₂—;
R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR¹⁶, —NR¹⁴(CO)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;
R⁸ is hydrogen, (C₁-C₆)alkyl, aryl (C₁-C₆)alkyl, —C(O)R¹⁴ or —COOR¹⁴;
R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;
R¹⁴ and R¹⁵ are independently selected from the group consisting of hydrogen, (C₁-C₆)alkyl, aryl and aryl-substituted (C₁-C₆)alkyl;
R¹⁶ is (C₁-C₆)alkyl, aryl or R¹⁷-substituted aryl;
R¹⁸ is hydrogen or (C₁-C₆)alkyl; and
R¹⁹ is hydrogen, hydroxy or (C₁-C₆)alkoxy, to regulate the amount of ApoE isoform 4 in the bloodstream and/or brain of the subject.

53. A method of preventing, treating, or ameliorating symptoms of Alzheimer's Disease comprising the step of administering to a subject in need of such treatment an effective amount of a composition comprising at least one compound represented by Formula (V): 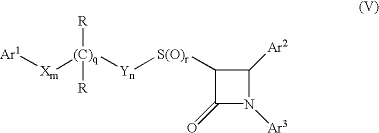
or a pharmaceutically acceptable salt thereof or solvate thereof, wherein, in Formula (V) above:
Ar¹ is aryl, R¹⁰-substituted aryl or heteroaryl;
Ar² is aryl or R⁴-substituted aryl;
Ar³ is aryl or R⁵-substituted aryl;
X and Y are independently selected from the group consisting of —CH₂—, —CH(lower alkyl)- and —C(dilower alkyl)-;
R is —OR⁶, —O(CO)R⁶, —O(CO)OR⁹ or —O(CO)NR⁶R⁷; R¹ is hydrogen, lower alkyl or aryl; or R and R¹ together are ═O;
q is 0 or 1;
r is 0, 1 or 2;
m and n are independently 0, 1, 2, 3, 4 or 5; provided that the sum of m, n and q is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl;
R⁹ is lower alkyl, aryl or aryl-substituted lower alkyl; and
R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, —CF₃, —CN, —NO₂ and halogen to prevent, treat, or ameliorate symptoms of Alzheimer's Disease in the subject.

54. A method of regulating the production of at least one amyloid β peptide in a subject or regulating a level of at least one amyloid β peptide in bloodstream and/or brain of a subject, comprising the step of administering to a subject in need of such treatment an effective amount of a composition comprising at least one compound represented by Formula (V): 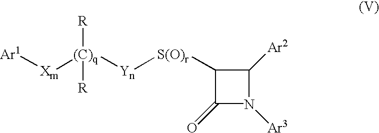
or a pharmaceutically acceptable salt thereof or solvate thereof, wherein, in Formula (V) above:
Ar¹ is aryl, R¹⁰-substituted aryl or heteroaryl;
Ar² is aryl or R⁴-substituted aryl;
Ar³ is aryl or R⁵-substituted aryl;
X and Y are independently selected from the group consisting of —CH₂—, —CH(lower alkyl)- and —C(dilower alkyl)-;
R is —OR⁶, —O(CO)R⁶, —O(CO)OR⁹ or —O(CO)NR⁶R⁷; R¹ is hydrogen, lower alkyl or aryl; or R and R¹ together are ═O;
q is 0 or 1;
r is 0, 1 or 2;
m and n are independently 0, 1, 2, 3, 4 or 5; provided that the sum of m, n and q is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl;
R⁹ is lower alkyl, aryl or aryl-substituted lower alkyl; and
R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, —CF₃, —CN, —NO₂ and halogen to regulate the production or level of at least one amyloid β peptide in the subject.

55. A method of regulating the amount of ApoE isoform 4 in the bloodstream and/or brain of the subject comprising the step of administering to a subject in need of such treatment an effective amount of a composition comprising at least one compound represented by Formula (V): 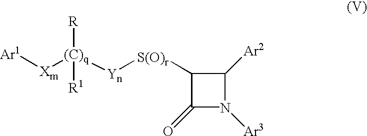
or a pharmaceutically acceptable salt thereof or solvate thereof, wherein, in Formula (V) above:
Ar¹ is aryl, R¹⁰-substituted aryl or heteroaryl;
Ar² is aryl or R⁴-substituted aryl;
Ar³ is aryl or R⁵-substituted aryl;
X and Y are independently selected from the group consisting of —CH₂—, —CH(lower alkyl)- and —C(dilower alkyl)-;
R is —OR⁶, —O(CO)R⁶, —O(CO)OR⁹ or —O(CO)NR⁶R⁷; R¹ is hydrogen, lower alkyl or aryl; or R and R¹ together are ═O;
q is 0 or 1;
r is 0, 1 or 2;
m and n are independently 0, 1, 2, 3, 4 or 5; provided that the sum of m, n and q is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl;
R⁹ is lower alkyl, aryl or aryl-substituted lower alkyl; and
R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶,
—O(CH₂)₁₋₁₀CONR⁶R⁷, —CF₃, —CN, —NO₂ and halogen to regulate the amount of ApoE isoform 4 in the bloodstream and/or brain of the subject.

56. A method of preventing, treating, or ameliorating symptoms of Alzheimer's Disease comprising the step of administering to a subject in need of such treatment an effective amount of a composition comprising at least one compound Ho represented by Formula (VI): 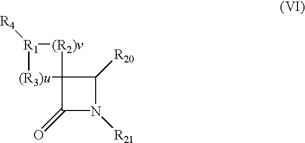
or a pharmaceutically acceptable salt thereof or solvate thereof, wherein:
R₁ is 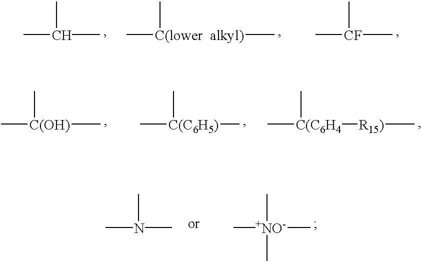
R₂ and R₃ are independently selected from the group consisting of: —CH₂—, —CH(lower alkyl)-, —C(di-lower alkyl)-, —CH═CH— and —C(lower alkyl)=CH—; or R₁ together with an adjacent R₂, or R₁ together with an adjacent R₃, form a —CH═CH— or a —CH═C(lower alkyl)- group;
u and v are independently 0, 1, 2 or 3, provided both are not zero; provided that when R₂ is —CH═CH— or —C(lower alkyl)=CH—, v is 1; provided that when R₃ is —CH═CH— or —C(lower alkyl)=CH—, u is 1; provided that when v is 2 or 3, the R₂'s can be the same or different; and provided that when u is 2 or 3, the R₃'s can be the same or different;
R₄ is selected from B—(CH₂)ₘC(O)—, wherein m is 0, 1, 2, 3, 4 or 5;
B—(CH₂)_{q}—, wherein q is 0, 1, 2, 3, 4, 5 or 6;
B—(CH₂)ₑ-Z-(CH₂)ᵣ—, wherein Z is —O—, —C(O)—, phenylene, —N(R₈)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6;
B—(C₂-C₆ alkenylene)-;
B—(C₄-C₆ alkadienylene)-;
B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6;
B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6;
B—(CH₂)ₜ—V—(C₂-C₆ alkenylene)- or
B—(C₂-C₆ alkenylene)—V—(CH₂)ₜ—, wherein V and t are as defined above, provided that the sum of t and the number of carbon atoms in the alkenylene chain is 2, 3, 4, 5 or 6;
B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or
T-(CH₂)ₛ—, wherein T is cycloalkyl of 3-6 carbon atoms and s is 0, 1, 2, 3, 4, 5 or 6; or
R₁ and R₄ together form the group 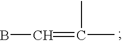
B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or 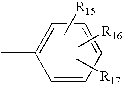
W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R₇-benzyl, benzyloxy, R₇-benzyloxy, phenoxy, R₇-phenoxy, dioxolanyl, NO₂, —N(R₈)(R₉), N(R₈)(R₉)-lower alkylene-, N(R₈)(R₉)-lower alkylenyloxy-, OH, halogeno, —CN, —N₃, —NHC(O)OR₁₀, —NHC(O)R₁₀, R₁₁O₂SNH—, (R₁₁O₂S)₂N—, —S(O)₂NH₂, —S(O)₀₋₂R₈, tert-butyldimethyl-silyloxymethyl, —C(O)R₁₂, —COOR₁₉, —CON(R₈)(R₉), —CH═CHC(O)R₁₂, -lower alkylene-C(O)R₁₂, R₁₀C(O)(lower alkylenyloxy)-, N(R₈)(R₉)C(O)(lower alkylenyloxy)- and 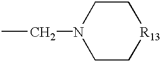
for substitution on ring carbon atoms, and the substituents on the substituted heteroaryl ring nitrogen atoms, when present, are selected from the group consisting of lower alkyl, lower alkoxy, —C(O)OR₁₀, —C(O)R₁₀, OH, N(R₈)(R₉)-lower alkylene-, N(R₈)(R₉)-lower alkylenyloxy-, —S(O)₂NH₂ and 2-(trimethylsilyl)-ethoxymethyl;
R₇ is 1-3 groups independently selected from the group consisting of lower alkyl, lower alkoxy, —COOH, NO₂, —N(R₈)(R₉), OH, and halogeno;
R₈ and R₉ are independently selected from H or lower alkyl;
R₁₀ is selected from lower alkyl, phenyl, R₇-phenyl, benzyl or R₇-benzyl;
R₁₁ is selected from OH, lower alkyl, phenyl, benzyl, R₇-phenyl or R₇-benzyl;
R₁₂ is selected from H, OH, alkoxy, phenoxy, benzyloxy, 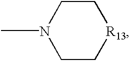
—N(R₈)(R₉), lower alkyl, phenyl or R₇-phenyl;
R₁₃ is selected from —O—, —CH₂—, —NH—, —N(lower alkyl)- or —NC(O)R₁₉;
R₁₅, R₁₆ and R₁₇ are independently selected from the group consisting of H and the groups defined for W; or R₁₅ is hydrogen and R₁₆ and R₁₇, together with adjacent carbon atoms to which they are attached, form a dioxolanyl ring;
R₁₉ is H, lower alkyl, phenyl or phenyl lower alkyl; and
R₂₀ and R₂₁ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above, to prevent, treat, or ameliorate symptoms of Alzheimer's Disease in the subject.

57. A method of regulating the production of at least one amyloid β peptide in a subject or regulating a level of at least one amyloid β peptide in bloodstream and/or brain of a subject, comprising the step of administering to a subject in need of such treatment an effective amount of a composition comprising at least one compound represented by Formula (VI): 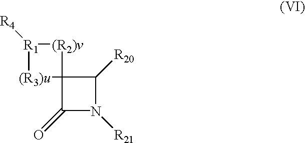
or a pharmaceutically acceptable salt thereof or solvate thereof, wherein:
R₁ is 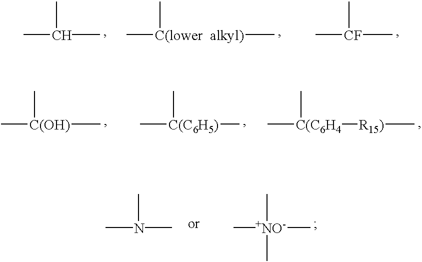
R₂ and R₃ are independently selected from the group consisting of: —CH₂—, —CH(lower alkyl)-, —C(di-lower alkyl)-, —CH═CH— and —C(lower alkyl)=CH—; or R₁ together with an adjacent R₂, or R₁ together with an adjacent R₃, form a —CH═CH— or a —CH═C(lower alkyl)- group;
u and v are independently 0, 1, 2 or 3, provided both are not zero; provided that when R₂ is —CH═CH— or —C(lower alkyl)=CH—, v is 1; provided that when R₃ is —CH═CH— or —C(lower alkyl)=CH—, u is 1; provided that when v is 2 or 3, the R₂'s can be the same or different; and provided that when u is 2 or 3, the R₃'s can be the same or different;
R₄ is selected from B—(CH₂)ₘC(O)—, wherein m is 0, 1, 2, 3, 4 or 5;
B—(CH₂)_{q}—, wherein q is 0, 1, 2, 3, 4, 5 or 6;
B—(CH₂)ₑ-Z-(CH₂)ᵣ—, wherein Z is —O—, —C(O)—, phenylene, —N(R₈)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6;
B—(C₂-C₆ alkenylene)-;
B—(C₄-C₆ alkadienylene)-;
B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6;
B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6;
B—(CH₂)ₜ—V—(C₂-C₆ alkenylene)- or
B—(C₂-C₆ alkenylene)—V—(CH₂)ₜ—, wherein V and t are as defined above, provided that the sum of t and the number of carbon atoms in the alkenylene chain is 2, 3, 4, 5 or 6;
B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or
T-(CH₂)ₛ—, wherein T is cycloalkyl of 3-6 carbon atoms and s is 0, 1, 2, 3, 4, 5 or 6; or
R₁ and R₄ together form the group 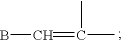
B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or 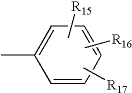
W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R₇-benzyl, benzyloxy, R₇-benzyloxy, phenoxy, R₇-phenoxy, dioxolanyl, NO₂, —N(R₈)(R₉), N(R₈)(R₉)-lower alkylene-, N(R₈)(R₉)-lower alkylenyloxy-, OH, halogeno, —CN, —N₃, —NHC(O)OR₁₀, —NHC(O)R₁₀, R₁₁O₂SNH—, (R₁₁O₂S)₂N—, —S(O)₂NH₂, —S(O)₀₋₂R₈, tert-butyldimethyl-silyloxymethyl, —C(O)R₁₂, —COOR₁₉, —CON(R₈)(R₉), —CH═CHC(O)R₁₂, -lower alkylene-C(O)R₁₂, R₁₀C(O)(lower alkylenyloxy)-, N(R₈)(R₉)C(O)(lower alkylenyloxy)- and 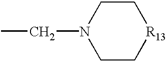
for substitution on ring carbon atoms, and the substituents on the substituted heteroaryl ring nitrogen atoms, when present, are selected from the group consisting of lower alkyl, lower alkoxy, —C(O)OR₁₀, —C(O)R₁₀, OH, N(R₈)(R₉)-lower alkylene-, N(R₈)(R₉)-lower alkylenyloxy-, —S(O)₂NH₂ and 2-(trimethylsilyl)-ethoxymethyl;
R₇ is 1-3 groups independently selected from the group consisting of lower alkyl, lower alkoxy, —COOH, NO₂, —N(R₈)(R₉), OH, and halogeno;
R₈ and R₉ are independently selected from H or lower alkyl;
R₁₀ is selected from lower alkyl, phenyl, R₇-phenyl, benzyl or R₇-benzyl;
R₁₁ is selected from OH, lower alkyl, phenyl, benzyl, R₇-phenyl or R₇-benzyl;
R₁₂ is selected from H, OH, alkoxy, phenoxy, benzyloxy, 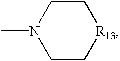
—N(R₈)(R₉), lower alkyl, phenyl or R₇-phenyl;
R₁₃ is selected from —O—, —CH₂—, —NH—, —N(lower alkyl)- or —NC(O)R₁₉;
R₁₅, R₁₆ and R₁₇ are independently selected from the group consisting of H and the groups defined for W; or R₁₅ is hydrogen and R₁₆ and R₁₇, together with adjacent carbon atoms to which they are attached, form a dioxolanyl ring;
R₁₉ is H, lower alkyl, phenyl or phenyl lower alkyl; and
R₂₀ and R₂₁ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above, to regulate the production or level of at least one amyloid β peptide in the subject.

58. A method of regulating the amount of ApoE isoform 4 in the bloodstream and/or brain of the subject comprising the step of administering to a subject in need of such treatment an effective amount of a composition comprising at least one compound represented by Formula (VI): 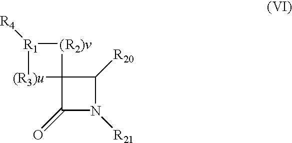
or a pharmaceutically acceptable salt thereof or solvate thereof, wherein:
R₁ is 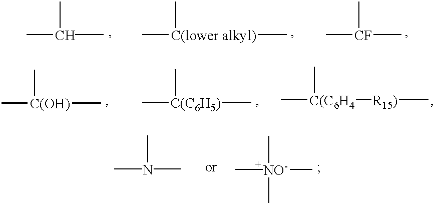
R₂ and R₃ are independently selected from the group consisting of: —CH₂—, —CH(lower alkyl)-, —C(d i-lower alkyl)-, —CH═CH— and —C(lower alkyl)=CH—; or R₁ together with an adjacent R₂, or R₁ together with an adjacent R₃, form a —CH═CH— or a —CH═C(lower alkyl)- group;
u and v are independently 0, 1, 2 or 3, provided both are not zero; provided that when R₂ is —CH═CH— or —C(lower alkyl)=CH—, v is 1; provided that when R₃ is —CH═CH— or —C(lower alkyl)=CH—, u is 1; provided that when v is 2 or 3, the R₂'s can be the same or different; and provided that when u is 2 or 3, the R₃'s can be the same or different;
R₄ is selected from B—(CH₂)ₘC(O)—, wherein m is 0, 1, 2, 3, 4 or 5;
B—(CH₂)_{q}—, wherein q is 0, 1, 2, 3, 4, 5 or 6;
B—(CH₂)ₑ-Z-(CH₂)ᵣ—, wherein Z is —O—, —C(O)—, phenylene, —N(R₈)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6;
B—(C₂-C₆ alkenylene)-;
B—(C₄-C₆ alkadienylene)-;
B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6;
B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6;
B—(CH₂)ₜ—V—(C₂-C₆ alkenylene)- or
B—(C₂-C₆ alkenylene)—V—(CH₂)ₜ—, wherein V and t are as defined above, provided that the sum of t and the number of carbon atoms in the alkenylene chain is 2, 3, 4, 5 or 6;
B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or
T-(CH₂)ₛ—, wherein T is cycloalkyl of 3-6 carbon atoms and s is 0, 1, 2, 3, 4, 5 or 6; or
R₁ and R₄ together form the group 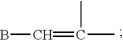
B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or 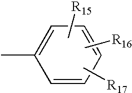
W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R₇-benzyl, benzyloxy, R₇-benzyloxy, phenoxy, R₇-phenoxy, dioxolanyl, NO₂, —N(R₈)(R₉), N(R₈)(R₉)-lower alkylene-, N(R₈)(R₉)-lower alkylenyloxy-, OH, halogeno, —CN, —N₃, —NHC(O)OR₁₀, —NHC(O)R₁₀, R₁₁O₂SNH—, (R₁₁O₂S)₂N—, —S(O)₂NH₂, —S(O)₀₋₂R₈, tert-butyldimethyl-silyloxymethyl, —C(O)R₁₂, —COOR₁₉, —CON(R₈)(R₉), —CH═CHC(O)R₁₂, -lower alkylene—C(O)R₁₂, R₁₀C(O)(lower alkylenyloxy)-, N(R₈)(R₉)C(O)(lower alkylenyloxy)- and 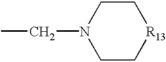
for substitution on ring carbon atoms, and the substituents on the substituted heteroaryl ring nitrogen atoms, when present, are selected from the group consisting of lower alkyl, lower alkoxy, —C(O)OR₁₀, —C(O)R₁₀, OH, N(R₈)(R₉)-lower alkylene-, N(R₈)(R₉)-lower alkylenyloxy-, —S(O)₂NH₂ and 2-(trimethylsilyl)-ethoxymethyl;
R₇ is 1-3 groups independently selected from the group consisting of lower alkyl, lower alkoxy, —COOH, NO₂, —N(R₈)(R₉), OH, and halogeno;
R₈ and R₉ are independently selected from H or lower alkyl;
R₁₀ is selected from lower alkyl, phenyl, R₇-phenyl, benzyl or R₇-benzyl;
R₁₁ is selected from OH, lower alkyl, phenyl, benzyl, R₇-phenyl or R₇-benzyl;
R₁₂ is selected from H, OH, alkoxy, phenoxy, benzyloxy, 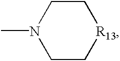
—N(R₈)(R₉), lower alkyl, phenyl or R₇-phenyl;
R₁₃ is selected from —O—, —CH₂—, —NH—, —N(lower alkyl)- or —NC(O)R₁₉;
R₁₅, R₁₆ and R₁₇ are independently selected from the group consisting of H and the groups defined for W; or R₁₅ is hydrogen and R₁₆ and R₁₇, together with adjacent carbon atoms to which they are attached, form a dioxolanyl ring;
R₁₉ is H, lower alkyl, phenyl or phenyl lower alkyl; and
R₂₀ and R₂₁ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above, to regulate the amount of ApoE isoform 4 in the bloodstream and/or brain of the subject.

59. A method of preventing, treating, or ameliorating symptoms of Alzheimer's Disease comprising the step of administering to a subject in need of such treatment an effective amount of a composition comprising at least one compound represented by Formula (VII): 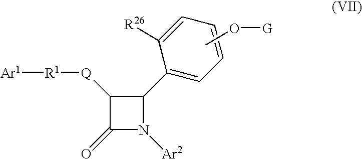
or a pharmaceutically acceptable salt thereof or solvate thereof, wherein, in Formula (VII) above,
R²⁶ is H or OG¹;
G and G¹ are independently selected from the group consisting of 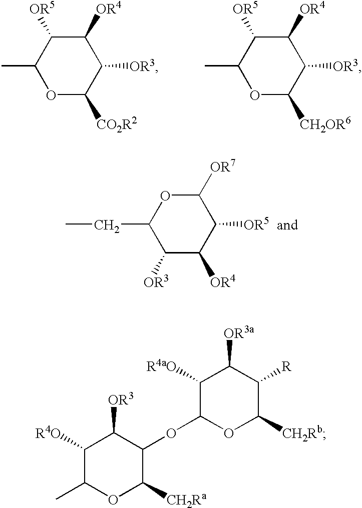
provided that when R²⁶ is H or OH, G is not H;
R, R^{a} and R^{b} are independently selected from the group consisting of H, —OH, halogeno, —NH₂, azido, (C₁-C₆)alkoxy(C₁-C₆)-alkoxy or —W—R³⁰;
W is independently selected from the group consisting of —NH—C(O)—, —O—C(O)—, —O—C(O)—N(R³¹)—, —NH—C(O)—N(R³¹)— and —O—C(S)—N(R³¹)—;
R² and R⁶ are independently selected from the group consisting of H, (C₁-C₆)alkyl, aryl and aryl(C₁-C₆)alkyl;
R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;
R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;
R³¹ is selected from the group consisting of H and (C₁-C₄)alkyl;
T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)-(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is a bond or, with the 3-position ring carbon of the azetidinone, forms the spiro group 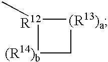
and
R¹ is selected from the group consisting of
—(CH₂)_{q}—, wherein q is 2-6, provided that when Q forms a spiro ring, q can also be zero or 1;
—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)_{g}—, wherein V is C₃-C₆ cycloalkylene, f is 1-5 and g is 0-5, provided that the sum of f and g is 1-6;
R¹² is 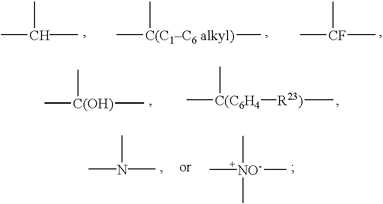
R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;
a and b are independently 0, 1, 2 or 3, provided both are not zero;
provided that when R¹³ is —CH═CH— or —C(C₁-C₆ alkyl)=CH—, a is 1;
provided that when R¹⁴ is —CH═CH— or —C(C₁-C₆ alkyl)=CH—, b is 1;
provided that when a is 2 or 3, the R¹³'s can be the same or different; and
provided that when b is 2 or 3, the R¹⁴'s can be the same or different;
and when Q is a bond, R¹ also can be: 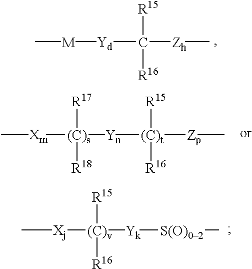
M is —O—, —S—, —S(O)— or —S(O)₂—;
X, Y and Z are independently selected from the group consisting of —CH₂—, —CH(C₁-C₆)alkyl- and —C(di-(C₁-C₆)alkyl);
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
R¹⁵ and R¹⁷ are independently selected from the group consisting of —OR¹⁹, —O(CO)R¹⁹, —O(CO)OR²¹ and —O(CO)NR¹⁹R²⁰;
R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;
d is 1, 2 or 3;
h is 0, 1, 2, 3 or 4;
s is 0 or 1; t is 0 or 1; m, n and p are independently 0-4;
provided that at least one of s and t is 1, and the sum of m, n, p, s and t is 1-6;
provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
and when Q is a bond and R¹ is 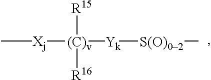
Ar¹ can also be pyridyl, isoxazolyl, furanyl, pyrrolyl, thienyl, imidazolyl, pyrazolyl, thiazolyl, pyrazinyl, pyrimidinyl or pyridazinyl;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy, to prevent, treat, or ameliorate symptoms of Alzheimer's Disease in the subject.

60. A method of regulating the production of at least one amyloid β peptide in a subject or regulating a level of at least one amyloid β peptide in bloodstream and/or brain of a subject, comprising the step of administering to a subject in need of such treatment an effective amount of a composition comprising at least one compound represented by Formula (VII): 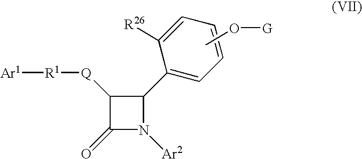
or a pharmaceutically acceptable salt thereof or solvate thereof, wherein, in Formula (VII) above,
R²⁶ is H or OG¹;
G and G¹ are independently selected from the group consisting of 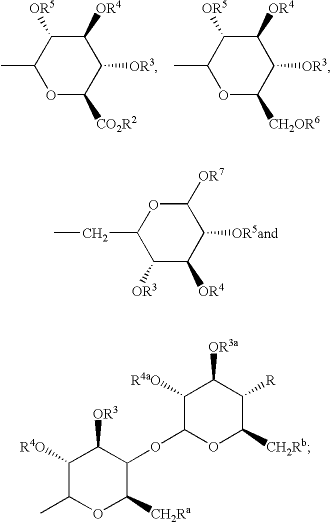
provided that when R²⁶ is H or OH, G is not H;
R, R^{a} and R^{b} are independently selected from the group consisting of H, —OH, halogeno, —NH₂, azido, (C₁-C₆)alkoxy(C₁-C₆)-alkoxy or —W—R³⁰;
W is independently selected from the group consisting of —NH—C(O)—, —O—C(O)—, —O—C(O)—N(R³¹)—, —NH—C(O)—N(R³¹)— and —O—C(S)—N(R³¹)—;
R² and R⁶ are independently selected from the group consisting of H, (C₁-C₆)alkyl, aryl and aryl(C₁-C₆)alkyl;
R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;
R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;
R³¹ is selected from the group consisting of H and (C₁-C₄)alkyl;
T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is a bond or, with the 3-position ring carbon of the azetidinone, forms the spiro group 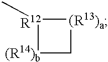
and
R¹ is selected from the group consisting of
—(CH₂)_{q}—, wherein q is 2-6, provided that when Q forms a spiro ring, q can also be zero or 1;
—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)_{g}—, wherein V is C₃-C₆ cycloalkylene, f is 1-5 and g is 0-5, provided that the sum of f and g is 1-6;
R¹² is 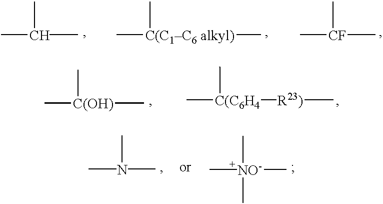
R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;
a and b are independently 0, 1, 2 or 3, provided both are not zero;
provided that when R¹³ is —CH═CH— or —C(C₁-C₆ alkyl)=CH—, a is 1;
provided that when R¹⁴ is —CH═CH— or —C(C₁-C₆ alkyl)=CH—, b is 1;
provided that when a is 2 or 3, the R¹³'s can be the same or different; and
provided that when b is 2 or 3, the R¹⁴'s can be the same or different;
and when Q is a bond, R¹ also can be: 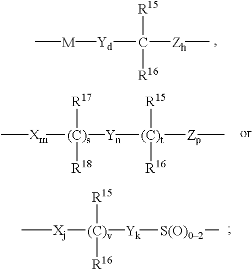
M is —O—, —S—, —S(O)— or —S(O)₂—;
X, Y and Z are independently selected from the group consisting of —CH₂—, —CH(C₁-C₆)alkyl- and —C(di-(C₁-C₆)alkyl);
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
R¹⁵ and R¹⁷ are independently selected from the group consisting of —OR¹⁹, —O(CO)R¹⁹, —O(CO)OR²¹ and —O(CO)NR¹⁹R²⁰;
R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;
d is 1, 2 or 3;
h is 0, 1, 2, 3 or 4;
s is 0 or 1; t is 0 or 1; m, n and p are independently 0-4;
provided that at least one of s and t is 1, and the sum of m, n, p, s and t is 1-6;
provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
and when Q is a bond and R¹ is 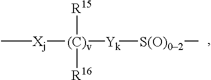
Ar¹ can also be pyridyl, isoxazolyl, furanyl, pyrrolyl, thienyl, imidazolyl, pyrazolyl, thiazolyl, pyrazinyl, pyrimidinyl or pyridazinyl;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy, to regulate the production or level of at least one amyloid β peptide in the subject.

61. A method of regulating the amount of ApoE isoform 4 in the bloodstream and/or brain of the subject comprising the step of administering to a subject in need of such treatment an effective amount of a composition comprising at least one compound represented by Formula (VII): 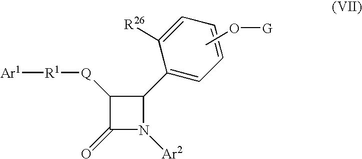
or a pharmaceutically acceptable salt thereof or solvate thereof, wherein, in Formula (VII) above,
R²⁶ is H or OG¹;
G and G¹ are independently selected from the group consisting of 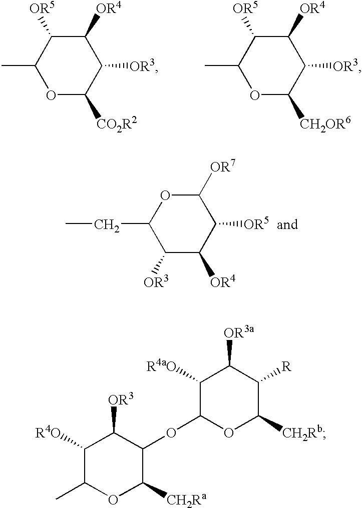
provided that when R²⁶ is H or OH, G is not H;
R, R^{a} and R^{b} are independently selected from the group consisting of H, —OH, halogeno, —NH₂, azido, (C₁-C₆)alkoxy(C₁-C₆)-alkoxy or —W—R³⁰;
W is independently selected from the group consisting of —NH—C(O)—, —O—C(O)—, —O—C(O)—N(R³¹)—, —NH—C(O)—N(R³¹)— and —O—C(S)—N(R³¹)—;
R² and R⁶ are independently selected from the group consisting of H, (C₁-C₆)alkyl, aryl and aryl(C₁-C₆)alkyl;
R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;
R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C7)cycloalkyl and R³²-substituted-(C₃-C7)cycloalkyl(C₁-C₆)alkyl;
R³¹ is selected from the group consisting of H and (C₁-C₄)alkyl;
T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is a bond or, with the 3-position ring carbon of the azetidinone, forms the spiro group 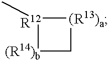
and
R¹ is selected from the group consisting of
—(CH₂)_{q}—, wherein q is 2-6, provided that when Q forms a spiro ring, q can also be zero or 1;
—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)_{g}—, wherein V is C₃-C₆ cycloalkylene, f is 1-5 and g is 0-5, provided that the sum of f and g is 1-6;
R¹² is 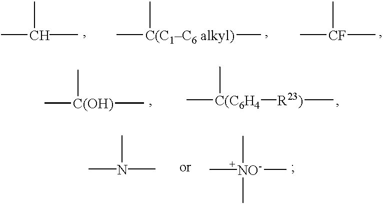
R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;
a and b are independently 0, 1, 2 or 3, provided both are not zero;
provided that when R¹³ is —CH═CH— or —C(C₁-C₆ alkyl)=CH—, a is 1;
provided that when R¹⁴ is —CH═CH— or —C(C₁-C₆ alkyl)=CH—, b is 1;
provided that when a is 2 or 3, the R¹³'s can be the same or different; and
provided that when b is 2 or 3, the R¹⁴'s can be the same or different;
and when Q is a bond, R¹ also can be: 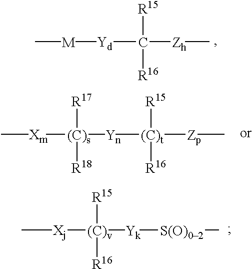
M is —O—, —S—, —S(O)— or —S(O)₂—;
X, Y and Z are independently selected from the group consisting of —CH₂—, —CH(C₁-C₆)alkyl- and —C(di-(C₁-C₆)alkyl);
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
R¹⁵ and R¹⁷ are independently selected from the group consisting of —OR¹⁹, —O(CO)R¹⁹, —O(CO)OR²¹ and —O(CO)NR¹⁹R²⁰;
R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;
d is 1, 2 or 3;
h is 0, 1, 2, 3 or 4;
s is 0 or 1; t is 0 or 1; m, n and p are independently 0-4;
provided that at least one of s and t is 1, and the sum of m, n, p, s and t is 1-6;
provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
and when Q is a bond and R¹ is 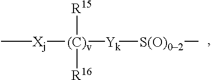
Ar¹ can also be pyridyl, isoxazolyl, furanyl, pyrrolyl, thienyl, imidazolyl, pyrazolyl, thiazolyl, pyrazinyl, pyrimidinyl or pyridazinyl;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy, to regulate the amount of ApoE isoform 4 in the bloodstream and/or brain of the subject.

62. A method of preventing, treating, or ameliorating symptoms of Alzheimer's Disease comprising the step of administering to a subject in need of such treatment an effective amount of a composition comprising at least one compound represented by Formula (VIII): 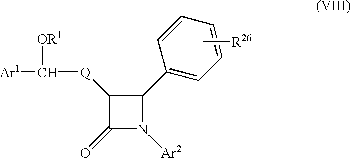
or a pharmaceutically acceptable salt or solvate thereof, wherein
R²⁶ is selected from the group consisting of:
a) OH;
b) OCH₃;
c) fluorine and
d) chlorine.
R¹ is selected from the group consisting of 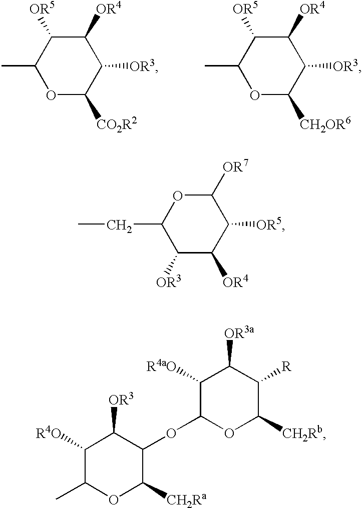
—SO₃H; natural and unnatural amino acids.
R, R^{a} and R^{b} are independently selected from the group consisting of H, —OH, halogeno, —NH₂, azido, (C₁-C₆)alkoxy(C₁-C₆)-alkoxy and —W—R³⁰;
W is independently selected from the group consisting of —NH—C(O)—, —O—C(O)—, —O—C(O)—N(R³¹)—, —NH—C(O)—N(R³¹)— and —O—C(S)—N(R³¹)—;
R² and R⁶ are independently selected from the group consisting of H, (C₁-C₆)alkyl, aryl and aryl(C₁-C₆)alkyl;
R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;
R³⁰ is independently selected form the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C7)cycloalkyl and R³²-substituted-(C₃-C7)cycloalkyl(C₁-C₆)alkyl;
R³¹ is independently selected from the group consisting of H and (C₁-C₄)alkyl;
T is independently selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of H, halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is —(CH₂)_{q}—, wherein q is 2-6, or, with the 3-position ring carbon of the azetidinone, forms the spiro group 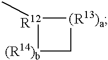
R¹² is 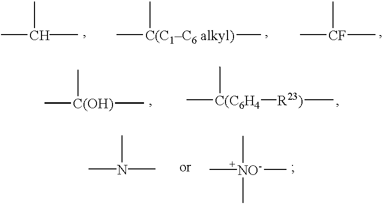
R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;
a and b are independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, the R¹³'s can be the same or different; and provided that when b is 2 or 3, the R¹⁴'s can be the same or different;
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy, to prevent, treat, or ameliorate symptoms of Alzheimer's Disease in the subject.

63. A method of regulating the production of at least one amyloid β peptide in a subject or regulating a level of at least one amyloid β peptide in bloodstream and/or brain of a subject, comprising the step of administering to a subject in need of such treatment an effective amount of a composition comprising at least one compound represented by Formula (VIII): 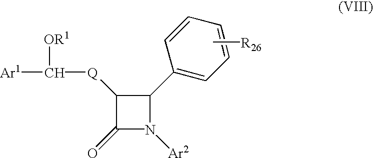
or a pharmaceutically acceptable salt or solvate thereof, wherein
R²⁶ is selected from the group consisting of:
a) OH;
b) OCH₃;
c) fluorine and
d) chlorine.
R¹ is selected from the group consisting of 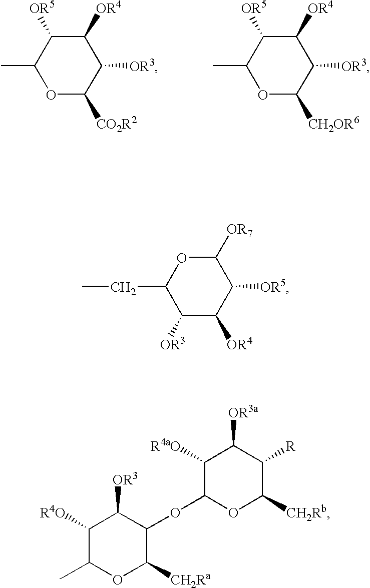
—SO₃H; natural and unnatural amino acids.
R, R^{a} and R^{b} are independently selected from the group consisting of H, —OH, halogeno, —NH₂, azido, (C₁-C₆)alkoxy(C₁-C₆)-alkoxy and —W—R³⁰;
W is independently selected from the group consisting of —NH—C(O)—, —O—C(O)—, —O—C(O)—N(R³¹)—, —NH—C(O)—N(R³¹)— and —O—C(S)—N(R³¹)—;
R² and R⁶ are independently selected from the group consisting of H, (C₁-C₆)alkyl, aryl and aryl(C₁-C₆)alkyl;
R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;
R³⁰ is independently selected form the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C7)cycloalkyl and R³²-substituted-(C₃-C7)cycloalkyl(C₁-C₆)alkyl;
R³¹ is independently selected from the group consisting of H and (C₁-C₄)alkyl;
T is independently selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of H, halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is —(CH₂)_{q}—, wherein q is 2-6, or, with the 3-position ring carbon of the azetidinone, forms the spiro group 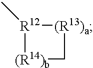
R¹² is 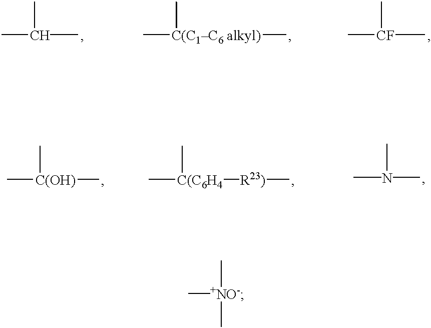
R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;
a and b are independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, the R¹³'s can be the same or different; and provided that when b is 2 or 3, the R¹⁴'s can be the same or different;
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
R¹⁹and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or—COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy, to regulate the production or level of at least one amyloid β peptide in the subject.

64. A method of regulating the amount of ApoE isoform 4 in the bloodstream and/or brain of the subject comprising the step of administering to a subject in need of such treatment an effective amount of a composition comprising at least one compound represented by Formula (VIII): 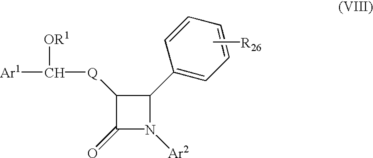
or a pharmaceutically acceptable salt or solvate thereof, wherein
R²⁶ is selected from the group consisting of:
a) OH;
b) OCH₃;
c) fluorine and
d) chlorine.
R¹ is selected from the group consisting of 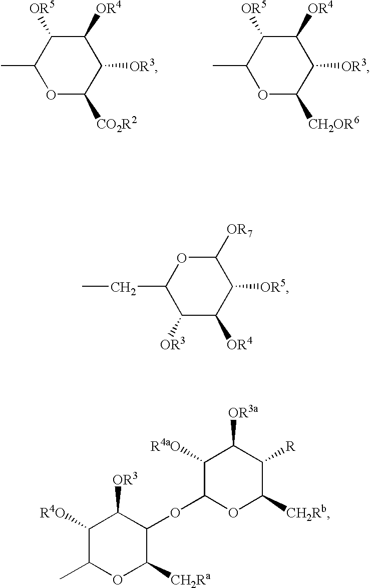
—SO₃H; natural and unnatural amino acids.
R, R^{a} and R^{b} are independently selected from the group consisting of H, —OH, halogeno, —NH₂, azido, (C₁-C₆)alkoxy(C₁-C₆)-alkoxy and —W—R³⁰;
W is independently selected from the group consisting of —NH—C(O)—, —O—C(O)—, —O—C(O)—N(R³¹)—, —NH—C(O)—N(R³¹)— and —O—C(S)N(R³¹)—;
R² and R⁶ are independently selected from the group consisting of H, (C₁-C₆)alkyl, aryl and aryl(C₁-C₆)alkyl;
R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;
R³⁰ is independently selected form the group consisting of R³²-substituted , R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl (C₁-C₆)alkyl;
R³¹ is independently selected from the group consisting of H and (C₁-C₄)alkyl;
T is independently selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of H, halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is —(CH₂)_{q}—, wherein q is 2-6, or, with the 3-position ring carbon of the azetidinone, forms the spiro group 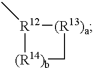
R¹² is 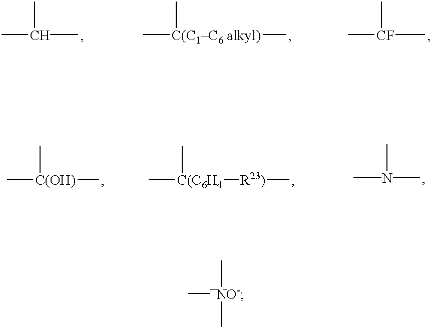
R¹³ and R¹⁴ are independently selected from the group consisting of —CH₂—, —CH(C₁-C₆ alkyl)-, —C(di-(C₁-C₆) alkyl), —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;
a and b are independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, the R¹³'s can be the same or different; and provided that when b is 2 or 3, the R¹⁴'s can be the same or different;
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy, to regulate the amount of ApoE isoform 4 in the bloodstream and/or brain of the subject.
